(19) United States
(12) Patent Application Publication
(10) Pub. No.: US 2026/0265762 A1
(43) Pub. Date: Sep. 10, 2026

(54) UNTRANSLATED REGION AND POLY(A) TAIL SEQUENCES FOR USE IN METHODS AND COMPOSITIONS FOR GENOME MODULATION

(71) Applicant: Tessera Therapeutics, Inc., Somerville, MA (US)

(72) Inventors: Chunxi ZENG, Somerville, MA (US); Xijia WANG, Somerville, MA (US); Anne Helen BOTHMER, Somerville, MA (US); Cecilia Giovanna Silvia COTTA-RAMUSINO, Somerville, MA (US)

(21) Appl. No.: 19/165,509

(22) PCT Filed: Mar. 14, 2024
(86) PCT No.: PCT/US2024/019968

§ 371 Date: Sep. 15, 2025

Related U.S. Application Data
(60) Provisional application No. 63/490,413, filed on Mar. 15, 2023.

(51) Int. Cl. C12N 15/67; C12N 9/12; C12N 9/22; C12N 15/11

(52) U.S. Cl. CPC C12N 15/67; C12N 9/1276; C12N 9/22; C12N 15/111; C07K 2319/80; C12N 2310/20; C12N 2830/50

(57) ABSTRACT

Artificial nucleic acid molecules comprising (1) at least one of; (a) a 3′-untranslated region (3′UTR) element and/or (b) a 5′-untranslated region (5′UTR) element: and (2) a poly(A) tail are described. The artificial nucleic acid molecules can further comprise a polypeptide comprising a reverse transcriptase (RT) domain and optionally an endonuclease domain for modifying a genome. Systems comprising the artificial nucleic acid molecules and methods of using the using the systems are also described.

### CROSS REFERENCE TO RELATED APPLICATION

This application claims priority to U.S. Provisional Application No. 63/490,413, filed Mar. 15, 2023, the disclosure of which is herein incorporated by reference in its entirety.

### FIELD OF THE INVENTION

The present invention is in the field of genome editing. More particularly, this invention relates to untranslated region (UTR) and poly(A) tail sequences for use in genome modification.

### REFERENCE TO SEQUENCE LISTING SUBMITTED ELECTRONICALLY

This application contains a sequence listing, which is submitted electronically. The contents of the electronic sequence listing (070992.3WO1 Sequence Listing.xml; size: 16,449,880 bytes; and creation date of Feb. 23, 2024) is herein incorporated by reference in its entirety.

### BACKGROUND

Integration of a nucleic acid of interest into a genome occurs at low frequency and with little site specificity, in the absence of a specialized protein to promote the insertion event. Integration of nucleic acids of interest into the genome can be limited by the expression levels of the gene modifying polypeptide delivering the nucleic acid of interest. Thus, there is a need in the art for improved methods and constructs for enhancing the expression of the gene modifying polypeptides capable of editing the host genomes.

### SUMMARY OF THE INVENTION

Provided herein are artificial ribonucleic acid (RNA) molecules for enhancing expression of a polypeptide comprising a reverse transcriptase (RT) domain and optionally an endonuclease domain. The RNA molecules can, for example, comprise (1) a nucleotide sequence encoding the polypeptide; (2) at least one of: (a) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269, and 8281; and/or (b) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or (3) a poly(A) tail for enhancing expression of the polypeptide, wherein the poly(A) tail comprises a nucleic acid sequence selected from the group consisting of SEQ ID NOs: 8282-8314.

In certain embodiments, the RNA molecules can, for example, comprise (1) a nucleotide sequence encoding the polypeptide; (2) at least one of: (a) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269, and 8281; and/or (b) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263.

In certain embodiments, the RNA molecules can, for example, comprise (1) a nucleotide sequence encoding the polypeptide; and (2) a poly(A) tail for enhancing expression of the polypeptide, wherein the poly(A) tail comprises a nucleic acid sequence selected from the group consisting of SEQ ID NOs: 8282-8314.

Also provided are artificial ribonucleic acid (RNA) molecules comprising: (1) at least one of: (a) a 3′-untranslated region (3′UTR) element comprising a nucleic acid sequence selected from the group consisting of SEQ ID NOs: 8201-8211; and/or (b) a 5′-untranslated region (5′UTR) element comprising a nucleic acid sequence selected form the group consisting of SEQ ID NOs: 8212-8247; and/or (2) a poly(A) tail for enhancing expression of the polypeptide, wherein the poly(A) tail comprises a nucleic acid sequence selected from the group consisting of SEQ ID NOs: 8282-8314.

In certain embodiments, the artificial ribonucleic acid (RNA) molecules comprise at least one of: (a) a 3′-untranslated region (3′UTR) element comprising a nucleic acid sequence selected from the group consisting of SEQ ID NOs: 8201-8211, 8256, 8264-8269, and 8281; and/or (b) a 5′-untranslated region (5′UTR) element comprising a nucleic acid sequence selected form the group consisting of SEQ ID NOs: 8212-8247 and 8259-8263.

In certain embodiments, the artificial ribonucleic acid (RNA) molecules comprise a poly(A) tail for enhancing expression of the polypeptide, wherein the poly(A) tail comprises a nucleic acid sequence selected from the group consisting of SEQ ID NOs: 8282-8314.

Also provided are systems for modifying DNA. The systems can, for example, comprise (a) an artificial ribonucleic acid (RNA) molecule for enhancing expression of a polypeptide comprising a reverse transcriptase (RT) domain and optionally an endonuclease domain, the artificial RNA molecule comprising: (1) a nucleotide sequence encoding the polypeptide; (2) at least one of: (i) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269 and 8281; and/or (ii) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or (3) a poly(A) tail for enhancing expression of the polypeptide, wherein the poly(A) tail comprises a nucleic acid sequence selected from the group consisting of SEQ ID NOs: 8282-8314; and (b) a template RNA (or DNA encoding the template RNA) comprising (e.g., from 5′ to 3′): (i) optionally a sequence that binds a target site in the DNA (e.g., a second strand of a site in a target genome); (ii) a sequence that binds the polypeptide; (iii) a heterologous object sequence; and (iv) optionally a 3′ target homology domain. The heterologous object sequence can, for example, comprise an alteration relative to a corresponding original sequence (e.g., a wild-type sequence), wherein the alteration improves the speed, fidelity, or speed and fidelity of target-primed reverse transcription by the reverse transcriptase.

Also provided are systems for modifying DNA. The systems can, for example, comprise (a) an artificial ribonucleic acid (RNA) molecule for enhancing expression of a polypeptide comprising a reverse transcriptase (RT) domain and optionally an endonuclease domain, the artificial nucleic acid molecule comprising: (1) a nucleotide sequence encoding the polypeptide; (2) at least one of: (i) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-69, and 8281; and/or (ii) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or (3) a poly(A) tail for enhancing expression of the polypeptide, wherein the poly(A) tail comprises a nucleic acid sequence selected from the group consisting of SEQ ID NOs: 8282-8314; and (b) a template RNA (or DNA encoding the template RNA) comprising (e.g., from 5′ to 3′): (i) optionally a sequence that binds a target site in the DNA (e.g., a second strand of a site in a target genome), (ii) a sequence that binds the polypeptide, (iii) a heterologous object sequence, and (iv) optionally a 3′ target homology domain. Preferably, the heterologous object sequence has one or both of the following characteristics: i) does not comprise self-complementary sequences, e.g., that form hairpin structures, e.g., under stringent conditions, or if a self-complementary sequence is present, it has one, two, or all of the following characteristics (1) each self-complementary sequence is no more than 10, 9, 8, 7, 6, 5, 4, or 3 nucleotides in length; (2) the self-complementary sequence forms a hairpin comprising arms of no longer than 10, 9, 8, 7, 6, 5, 4, or 3 nucleotides in length, or (3) the self-complementary sequence comprises at least 1, 2, 3, 4, or 5 positions of non-complementarity (e.g., mismatches or bulges) with its partner sequence, and (4) does not comprise a repetitive sequence (e.g., a single-, di-, or tri-nucleotide repetitive sequence) or if a repetitive sequence is present it is of no more than 12, 11, 10, 9, 8, 7, or 6 nucleotides in length.

In certain embodiments, the 3′UTR element for enhancing expression of the polypeptide consists of a nucleic acid sequence selected from the group consisting of SEQ ID NOs: 8201-8211, 8256, 8264-8269, and 8281. In certain embodiments, the 5′UTR element for enhancing expression of the polypeptide consists of a nucleic acid sequence selected form the group consisting of SEQ ID NOs: 8212-8247 and 8259-8263. In certain embodiments, the poly(A) tail for enhancing expression of the polypeptide consists of a nucleic acid sequence selected from the group consisting of SEQ ID NOs: 8282-8314.

In certain embodiments, the 3′UTR element of the RNA molecule comprises a nucleic acid sequence of SEQ ID NO: 8201, SEQ ID NO: 8204, SEQ ID NO: 8209, SEQ ID NO: 8211, SEQ ID NO: 8256, or SEQ ID NO: 8281. In certain embodiments, the 3′UTR element of the RNA molecule consists of a nucleic acid sequence of SEQ ID NO: 8201, SEQ ID NO: 8204, SEQ ID NO: 8209, SEQ ID NO: 8211, SEQ ID NO: 8256, or SEQ ID NO: 8281.

In certain embodiments, the 5′UTR element of the RNA molecule comprises a nucleic acid sequence of SEQ ID NO:8214, SEQ ID NO:8230, SEQ ID NO:8235, SEQ ID NO:8236, or SEQ ID NO:8243. In certain embodiments, the 5′UTR element of the RNA molecule consists of a nucleic acid sequence of SEQ ID NO:8214, SEQ ID NO:8230, SEQ ID NO: 8235, SEQ ID NO:8236, or SEQ ID NO:8243.

In certain embodiments, the poly(A) tail of the RNA molecule comprises a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314. In certain embodiments, the poly(A) tail of the RNA molecule consists of a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

In certain embodiments, the artificial RNA molecule comprises the 3′UTR element and the 5′UTR element, wherein (1) the 3′UTR element comprises SEQ ID NO: 8209 and the 5′UTR element comprises SEQ ID NO: 8236; (2) the 3′UTR element comprises SEQ ID NO: 8201 and the 5′UTR element comprises SEQ ID NO: 8236; (3) the 3′UTR element comprises SEQ ID NO: 8209 and the 5′UTR element comprises SEQ ID NO: 8214; (4) the 3′UTR element comprises SEQ ID NO: 8209 and the 5′UTR element comprises SEQ ID NO: 8243; (5) the 3′UTR element comprises SEQ ID NO: 8281 and the 5′UTR element comprises SEQ ID NO: 8259; or (6) the 3′UTR element comprises SEQ ID NO: 8281 and the 5′UTR element comprises SEQ ID NO: 8263. In certain embodiments, the artificial RNA molecule comprises the 3′UTR element and the 5′UTR element, wherein (1) the 3′UTR element consists of SEQ ID NO: 8209 and the 5′UTR element consists of SEQ ID NO: 8236; (2) the 3′UTR element consists of SEQ ID NO: 8201 and the 5′UTR element consists of SEQ ID NO: 8236; (3) the 3′UTR element consists of SEQ ID NO: 8209 and the 5′UTR element consists of SEQ ID NO: 8214; (4) the 3′UTR element consists of SEQ ID NO: 8209 and the 5′UTR element consists of SEQ ID NO: 8243; (5) the 3′UTR element consists of SEQ ID NO: 8281 and the 5′UTR element consists of SEQ ID NO: 8259; or (6) the 3′UTR element consists of SEQ ID NO: 8281 and the 5′UTR element consists of SEQ ID NO: 8263.

In certain embodiments, the artificial RNA molecule comprises the poly(A) tail, wherein the poly(A) tail comprises the nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314. In certain embodiments, the artificial RNA molecule comprises the poly(A) tail, wherein the poly(A) tail consists of the nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

In certain embodiments, the artificial RNA molecule comprises the 3′UTR element, the 5′UTR element, and the poly(A) tail, wherein (1) the 3′UTR element comprises SEQ ID NO: 8209 and the 5′UTR element comprises SEQ ID NO: 8236; (2) the 3′UTR element comprises SEQ ID NO: 8201 and the 5′UTR element comprises SEQ ID NO: 8236; (3) the 3′UTR element comprises SEQ ID NO: 8209 and the 5′UTR element comprises SEQ ID NO: 8214; (4) the 3′UTR element comprises SEQ ID NO: 8209 and the 5′UTR element comprises SEQ ID NO: 8243; (5) the 3′UTR element comprises SEQ ID NO: 8281 and the 5′UTR element comprises SEQ ID NO: 8259; or (6) the 3′UTR element comprises SEQ ID NO: 8281 and the 5′UTR element comprises SEQ ID NO: 8263; and the poly(A) tail comprises a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314. In certain embodiments, the artificial RNA molecule comprises the 3′UTR element, the 5′UTR element, and the poly(A) tail, wherein (1) the 3′UTR element consists of SEQ ID NO: 8209 and the 5′UTR element consists of SEQ ID NO: 8236; (2) the 3′UTR element consists of SEQ ID NO: 8201 and the 5′UTR element consists of SEQ ID NO: 8236; (3) the 3′UTR element consists of SEQ ID NO: 8209 and the 5′UTR element consists of SEQ ID NO: 8214; (4) the 3′UTR element consists of SEQ ID NO: 8209 and the 5′UTR element consists of SEQ ID NO: 8243; (5) the 3′UTR element consists of SEQ ID NO: 8281 and the 5′UTR element consists of SEQ ID NO: 8259; or (6) the 3′UTR element consists of SEQ ID NO: 8281 and the 5′UTR element consists of SEQ ID NO: 8263; and the poly(A) tail consists of a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

In certain embodiments, the artificial RNA molecule comprises the 3′UTR element, the 5′UTR element, and the poly(A) tail, wherein (1) the 3′UTR element comprises SEQ ID NO: 8209, the 5′UTR comprises SEQ ID NO: 8236, and the poly(A) tail comprises SEQ ID NO: 8306 or SEQ ID NO: 8307; (2) the 3′UTR element comprises SEQ ID NO: 8201, the 5′UTR element comprises SEQ ID NO: 8236, and the poly(A) tail comprises SEQ ID NO: 8306 or SEQ ID NO: 8307; (3) the 3′UTR element comprises SEQ ID NO: 8209, the 5′UTR element comprises SEQ ID NO: 8214, the poly(A) tail comprises SEQ ID NO: 8306 or SEQ ID NO: 8307; (4) the 3′UTR element comprises SEQ ID NO: 8209, the 5′UTR element comprises SEQ ID NO: 8243, and the poly(A) tail comprises SEQ ID NO: 8306 or SEQ ID NO: 8307; (5) the 3′UTR element comprises SEQ ID NO: 8281, the 5′UTR element comprises SEQ ID NO: 8259, the poly(A) tail comprises SEQ ID NO: 8306 or SEQ ID NO: 8307; or (6) the 3′UTR element comprises SEQ ID NO: 8281, the 5′UTR element comprises SEQ ID NO: 8263, and the poly(A) tail comprises SEQ ID NO: 8306 or SEQ ID NO: 8307. In certain embodiments, the artificial RNA molecule comprises the 3′UTR element, the 5′UTR element, and the poly(A) tail, wherein (1) the 3′UTR element comprises SEQ ID NO: 8209, the 5′UTR comprises SEQ ID NO: 8236, and the poly(A) tail comprises SEQ ID NO: 8306 or SEQ ID NO: 8307; (2) the 3′UTR element comprises SEQ ID NO: 8201, the 5′UTR element comprises SEQ ID NO: 8236, and the poly(A) tail comprises SEQ ID NO: 8306 or SEQ ID NO: 8307; (3) the 3′UTR element comprises SEQ ID NO: 8209, the 5′UTR element comprises SEQ ID NO: 8214, the poly(A) tail comprises SEQ ID NO: 8306 or SEQ ID NO: 8307; or (4) the 3′UTR element comprises SEQ ID NO: 8209, the 5′UTR element comprises SEQ ID NO: 8243, and the poly(A) tail comprises SEQ ID NO: 8306 or SEQ ID NO: 8307.

In certain embodiments, the artificial RNA molecule comprises the 3′UTR element, the 5′UTR element, and the poly(A) tail, wherein (1) the 3′UTR element comprises SEQ ID NO: 8201, the 5′UTR element comprises SEQ ID NO: 8236, and the poly(A) tail comprises SEQ ID NO:8307; (2) the 3′UTR element comprises SEQ ID NO: 8209, the 5′UTR element comprises SEQ ID NO: 8214, and the poly(A) tail comprises SEQ ID NO:8306. In certain embodiments, the artificial RNA molecule comprises the 3′UTR element, the 5′UTR element, and the poly(A) tail, wherein (1) the 3′UTR element consists of SEQ ID NO: 8201, the 5′UTR element consists of SEQ ID NO: 8236, and the poly(A) tail consists of SEQ ID NO: 8307; (2) the 3′UTR element consists of SEQ ID NO: 8209, the 5′UTR element consists of SEQ ID NO: 8214, and the poly(A) tail consists of SEQ ID NO:8306.

In certain embodiments, the artificial ribonucleic acid (RNA) molecule comprises (1) at least one of: (a) a 3′-untranslated region (3′UTR) element comprising a nucleic acid sequence selected from the group consisting of SEQ ID NOs: 8201-8211; and/or (b) a 5′-untranslated region (5′UTR) element comprising a nucleic acid sequence selected form the group consisting of SEQ ID NOs: 8212-8247; and/or (2) a poly(A) tail for enhancing expression of the polypeptide, wherein the poly(A) tail comprises a nucleic acid sequence selected from the group consisting of SEQ ID NOs: 8282-8314. In certain embodiments, the artificial RNA molecule comprises (1) at least one of: (a) a 3′UTR element consisting of a nucleic acid sequence selected from the group consisting of SEQ ID NOs: 8201-8211; and/or (b) a 5′UTR element consisting of a nucleic acid sequence selected from the group consisting of SEQ ID NOs: 8212-8247; and/or (2) a poly(A) tail for enhancing expression of the polypeptide, wherein the poly(A) tail consists of a nucleic acid sequence selected from the group consisting of SEQ ID NOs: 8282-8314.

In certain embodiments, the 3′UTR element of the artificial RNA molecule comprises a nucleic acid sequence of SEQ ID NO: 8201, SEQ ID NO: 8204, SEQ ID NO: 8209, or SEQ ID NO: 8211. In certain embodiments, the 3′UTR element of the artificial RNA molecule consists of a nucleic acid sequence of SEQ ID NO: 8201, SEQ ID NO: 8204, SEQ ID NO: 8209, or SEQ ID NO: 8211.

In certain embodiments, the 5′UTR element of the artificial RNA molecule comprises a nucleic acid sequence of SEQ ID NO: 8214, SEQ ID NO: 8230, SEQ ID NO: 8235, SEQ ID NO: 8236, or SEQ ID NO: 8243. In certain embodiments, the 5′UTR element of the artificial RNA molecule consists of a nucleic acid sequence of SEQ ID NO: 8214, SEQ ID NO: 8230, SEQ ID NO: 8235, SEQ ID NO: 8236, or SEQ ID NO: 8243.

In certain embodiments, the poly(A) tail of the RNA molecule comprises a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314. In certain embodiments, the poly(A) tail of the RNA molecule consists of a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

In certain embodiments, the artificial RNA molecule comprises the 3′UTR element and the 5′UTR element, wherein (1) the 3′UTR element comprises SEQ ID NO: 8201 and the 5′UTR element comprises SEQ ID NO: 8236; or (2) the 3′UTR element comprises SEQ ID NO: 8209 and the 5′UTR element comprises SEQ ID NO: 8214. In certain embodiments, the artificial RNA molecule comprises the 3′UTR element and the 5′UTR element, wherein (1) the 3′UTR element consists of SEQ ID NO: 8201 and the 5′UTR element consists of SEQ ID NO: 8236; or (2) the 3′UTR element consists of SEQ ID NO: 8209 and the 5′UTR element consists of SEQ ID NO: 8214.

In certain embodiments, the artificial RNA molecule comprises the poly(A) tail, wherein the poly(A) tail comprises the nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314. In certain embodiments, the artificial RNA molecule comprises the poly(A) tail, wherein the poly(A) tail consists of the nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

In certain embodiments, the artificial RNA molecule comprises the 3′UTR element, the 5′UTR element, and the poly(A) tail, wherein (1) the 3′UTR element comprises SEQ ID NO: 8201 and the 5′UTR element comprises SEQ ID NO: 8236; or (2) the 3′UTR element comprises SEQ ID NO: 8209 and the 5′UTR element comprises SEQ ID NO: 8214; and the poly(A) tail comprises a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314. In certain embodiments, the artificial RNA molecule comprises the 3′UTR element, the 5′UTR element, the poly(A) tail, wherein (1) the 3′UTR element consists of SEQ ID NO: 8201 and the 5′UTR element consists of SEQ ID NO: 8236; or (2) the 3′UTR element consists of SEQ ID NO: 8209 and the 5′UTR element consists of SEQ ID NO: 8214; and the poly(A) tail consists of a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

In certain embodiments, the artificial RNA molecule comprises the 3′UTR element, the 5′UTR element, and the poly(A) tail, wherein (1) the 3′UTR element comprises SEQ ID NO: 8201, the 5′UTR element comprises SEQ ID NO: 8236, and the poly(A) tail comprises SEQ ID NO:8307; (2) the 3′UTR element comprises SEQ ID NO: 8209, the 5′UTR element comprises SEQ ID NO: 8214, and the poly(A) tail comprises SEQ ID NO:8306. In certain embodiments, the artificial RNA molecule comprises the 3′UTR element, the 5′UTR element, and the poly(A) tail, wherein (1) the 3′UTR element consists of SEQ ID NO: 8201, the 5′UTR element consists of SEQ ID NO: 8236, and the poly(A) tail consists of SEQ ID NO: 8307; (2) the 3′UTR element consists of SEQ ID NO: 8209, the 5′UTR element consists of SEQ ID NO: 8214, and the poly(A) tail consists of SEQ ID NO:8306.

In certain embodiments, the polypeptide is a heterologous gene modifying polypeptide or a retrotransposon gene modifying polypeptide. In certain embodiments, the polypeptide comprises the reverse transcriptase domain and the endonuclease domain. The endonuclease domain can, for example, be a nickase domain, such as a Cas9 domain selected from SpCas9 domain, a BlatCas9 domain, a Nme2 Cas9 domain, a PnpCas9 domain, a SauCas9 domain, a SauCas9-KKH domain, a SauriCas9 domain, a SauriCas9-KKH domain, a ScaCas9-Sc++ domain, a SpyCas9 domain, a SpyCas9-NG domain, a SpyCas9-SpRY domain, or a St1Cas9 domain. In certain embodiments, the Cas9 domain comprising an N670A mutation, an N611A mutation, an N605A mutation, an N580A mutation, an N588A mutation, an N872A mutation, an N863A mutation, an N622A mutation, or an H840A mutation.

The reverse transcriptase domain can, for example, be selected from a retrovirus transcriptase domain. In certain embodiments, the retrovirus reverse transcriptase domain is a gamma retrovirus-derived reverse transcriptase domain. The gamma retrovirus-derived reverse transcriptase domain can, for example, comprise an amino acid sequence of a reverse-transcriptase domain sequence from a family selected from AVIRE, BAEVM, FFV, FLV, FOAMV, GALV, KORV, MLVAV, MLVBM, MLVCB, MLVFF, MLVMS, PERV, SFV1, SFV3L, WMSV, and XMRV6. In certain embodiments, the gamma retrovirus-derived reverse transcriptase domain is not derived from PERV.

In certain embodiments, the reverse transcriptase domain comprises one, two, three, four, five, six or more mutations corresponding to the following mutations D200N, L603W, T330P, D524G, E562Q, D583N, P51L, S67R, T197A, H204R, E302K, F309N, W313F, L435G, N454K, H594Q, L671P, E69K, or D653N in the reverse transcriptase domain of a murine leukemia virus reverse transcriptase.

In certain embodiments, the gene modifying polypeptide comprises the amino acid sequence of SEQ ID NO:8257 or SEQ ID NO: 8371.

In certain embodiments, the template RNA further comprises a reverse transcriptase (RT) terminator sequence situated between the heterologous object sequence and either (i) or (ii).

In certain embodiments, the heterologous object sequence encodes a target polypeptide or portion thereof or comprises a sequence that is the reverse complement of a sequence encoding the target polypeptide or portion thereof.

In certain embodiments, the polypeptide comprises the reverse transcriptase domain and the endonuclease domain, and the endonuclease domain is a Cas9 domain, and the template RNA comprises (i) a gRNA spacer that is complementary to a first portion of a target gene, and optionally comprises one or more consecutive nucleotides starting with the 3′ end of the flanking nucleotides of the gRNA spacer; (ii) a gRNA scaffold that binds to the Cas9 domain; (iii) a heterologous object sequence comprising a mutation region to introduce a mutation into (e.g., to correct a mutation in) a second portion of the target gene (wherein optionally the heterologous sequence comprises, from 5′ to 3′ a post-edit homology region, a mutation region, and a pre-edit homology region), and (iv) a primer binding site (PBS) sequence comprising at least 5, 6, 7, or 8 bases with 100% identity to a third portion of the target gene.

In certain embodiments, the target gene is a human PAH gene, and the template RNA comprises (i) a gRNA spacer that is complementary to a first portion of the human PAH gene, wherein the gRNA spacer has a sequence comprising the core nucleotides of a gRNA spacer sequence, preferably of Table 1A, Table 1B, Table 1C, or Table 1D in WO2023039435, which is herein incorporated by reference in its entirety, and optionally comprises one or more consecutive nucleotides starting with the 3′ end of the flanking nucleotides of the gRNA spacer, or wherein the gRNA spacer has a sequence of a spacer chosen from Tables 5A-5F, 8A-8D, E3, E3A, BB, E5, E5A, E6, or E6A in WO2023039435, which is herein incorporated by reference in its entirety; (ii) a gRNA scaffold that binds to the Cas9 domain; (iii) a heterologous object sequence comprising a mutation region to introduce a mutation into (e.g., to correct a mutation in) a second portion of the human PAH gene (wherein optionally the heterologous object sequence comprises, from 5′ to 3′, a post-edit homology region, a mutation region, and a pre-edit homology region); and (iv) a primer binding site (PBS) sequence comprising at least 5, 6, 7, or 8 bases with 100% identity to a third portion of the human PAH gene.

In certain embodiments, the template RNA consists of the sequence of SEQ ID NO: 8258 (RNACS7570); SEQ ID NO: 8320 (RNACS229); SEQ ID NO: 8321 (RNACS1515); or SEQ ID NO: 8372.

In certain embodiments, the reverse transcriptase domain and the endonuclease domain are linked by a peptide linker.

In certain embodiments, the target site is in a human genome.

Also provided are reaction mixtures comprising a cell and a system of the instant invention. In certain embodiments, the cell is a T cell (e.g., a primary T cell).

Also provided are reaction mixtures comprising a DNA comprising a target site and a system of the instant invention.

In certain embodiments, the artificial RNA molecule comprises one or more chemically modified nucleotides.

Also provided are deoxyribonucleic acid (DNA) molecules encoding an artificial RNA molecule of the instant invention.

Also provided are pharmaceutical compositions comprising an artificial RNA molecule of the invention, a system of the invention, or one or more nucleic acids encoding the same, and a pharmaceutically acceptable excipient or carrier. The pharmaceutically acceptable excipient or carrier can, for example, be selected from the group consisting of a plasmid vector, a viral vector, a vesicle, and a lipid nanoparticle. In certain embodiments, the viral vector is an adeno-associated virus.

Also provided are host cells (e.g., a mammalian cell, e.g., a human cell) comprising the artificial RNA molecule or the system or the DNA of the invention. The host cell can, for example, be a T cell (e.g., a primary T cell).

Also provided are methods of making the artificial RNA molecule of the invention. The methods comprise synthesizing the template RNA by in vitro transcription (e.g., solid state synthesis) or by introducing a DNA encoding the artificial RNA into a host cell under conditions that allow for the production of the template RNA.

Also provided are kits. The kits can, for example, comprise (a) a system, a reaction mixture, a DNA molecule, or a pharmaceutical composition of the invention; and (b) instructions for using the system, the reaction mixture, the DNA molecule, or the pharmaceutical composition.

Also provided are lipid nanoparticles (LNPs) comprising an artificial RNA molecule of the invention or a system of the invention.

Also provided are methods for modifying a target site in genomic DNA in a cell. The methods comprise contacting the cell with the system of the invention or one or more RNAs encoding the system of the invention, thereby modifying the target site in the genomic DNA in a cell.

Also provided are methods for treating a subject having a disease or condition associated with a genetic defect. The methods comprise administering to the subject the system of the invention, thereby treating the subject having a disease or condition associated with a genetic defect.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings.

FIG. 1 is a graph of % GFP positive cells obtained after nucleofecting U2OS-BFP cells with mRNAs that encoded the gene modifying polypeptide without the HiBiT protein tag (mRNAs encoding non-HiBiT tagged gene modifying polypeptides) following 4 days.

FIG. 2 is a graph of % GFP positive U2OS-BFP cells obtained after nucleofecting mRNAs that encoded the gene modifying peptide fused with the HiBiT protein tag (mRNAs encoding HiBiT-tagged gene modifying polypeptides) following 4 days.

FIG. 3 is a graph of the expression of the mRNAs encoding the HiBiT-tagged gene modifying polypeptides from mRNAs with reference 5′ UTRs at 6 hours post-nucleofection in U2OS-naïve cells (do not express BFP).

FIG. 4 is a graph of the expression levels of the HiBiT-tagged gene modifying polypeptides in Lonza's cryopreserved mouse hepatocytes (catalog number MCCP01) at 24 hours post-nucleofection.

FIG. 5 is a graph of the expression levels of the HiBiT-tagged gene modifying polypeptides from mRNAs in hepatocytes freshly harvested from wild-type C57BL/6 mice at 24 hours post-nucleofection.

FIG. 6 is a graph of % GFP positive cells obtained 4 days post-nucleofecting U2OS-BFP cells with mRNAs that encoded the gene modifying polypeptide without the HiBiT protein tag (mRNAs encoding non-HiBiT tagged gene modifying polypeptides).

FIG. 7 is a graph of % GFP positive cells 4 days post-nucleofecting mRNAs that encoded the gene modifying peptide fused with the HiBiT protein tag (mRNAs encoding HiBiT-tagged gene modifying polypeptides).

FIG. 8 is a graph of the expression of the HiBiT-tagged gene modifying polypeptides from mRNAs with reference 3′ UTRs at 6 hours post-nucleofection in U2OS-naïve cells (do not express BFP).

FIG. 9 is a graph of the expression levels of the HiBiT-tagged gene modifying polypeptides from mRNAs in Lonza's cryopreserved mouse hepatocytes following 24 hours post-nucleofection. The dotted line marks the expression from the mRNA with Reference 1 3′ UTR.

FIG. 10 is a graph of the expression levels of the HiBiT-tagged gene modifying polypeptides from mRNAs in hepatocytes freshly harvested from wild-type C57BL/6 mice 24 hours post-nucleofection. The dotted line marks the expression from the mRNA with Reference 1 3′ UTR.

FIG. 11 is a graph of % GFP positive cells obtained on day 4 after nucleofecting U2OS-BFP cells with mRNAs that encoded the gene modifying polypeptide without the HiBiT protein tag (mRNAs encoding non-HiBiT tagged gene modifying polypeptides), where the mRNA comprises various 5′ UTRs. The dotted line marks the GFP % produced by using gene modifying systems comprising mRNA comprising reference 5′UTR Reference 1.

FIG. 12 is a graph of % GFP positive cells obtained at day 4 after nucleofecting U2OS-BFP cells with mRNAs that encoded the HiBiT-tagged gene modifying polypeptide (mRNAs encoding HiBiT-tagged gene modifying polypeptides). The dotted line marks the GFP % produced by using gene modifying systems comprising mRNA comprising reference 5′UTR Reference 1.

FIG. 13 is a graph of the expression of the HiBiT-tagged gene modifying polypeptides from mRNAs comprising the various 5′ UTRs at 6 hours post-nucleofection in U2OS-naïve cells (do not express BFP).

FIG. 14 is a graph plotting HiBiT-tagged gene modifying polypeptide expression in U2OS-naïve cells (do not express BFP) from mRNAs comprising the various 5′ UTRs (normalized to expression from mRNAs comprising Reference 1 5′UTR) against % GFP obtained from nucleofecting gene modifying systems comprising the same HiBiT-tagged gene modifying polypeptides. Expression levels were obtained 6 hours post-nucleofection.

FIG. 15 is a graph of expression of the HiBiT-tagged gene modifying polypeptide from mRNAs comprising the various 5′ UTRs at 24 hours post-nucleofection in Lonza's cryopreserved mouse hepatocytes (catalog number MCCP01). The dotted line marks the expression level of the gene modifying polypeptide from mRNA Control 1 5′ UTR.

FIG. 16 is a graph of expression of the HiBiT-tagged gene modifying polypeptide from mRNAs comprising the various 5′ UTRs at 24 hours post-nucleofection in hepatocytes freshly harvested from wild-type C57BL/6 mice and normalized to the expression level of gene modifying polypeptide from an mRNA comprising Reference 1 5′UTR (dotted line).

FIG. 17 is a graph of % GFP positive cells obtained on day 4 after nucleofecting U2OS-BFP cells with mRNAs that encoded the gene modifying polypeptide without the HiBiT protein tag (mRNAs encoding non-HiBiT tagged gene modifying polypeptides), where the mRNA comprised various 3′ UTRs. The dotted line marks the GFP % produced by using gene modifying systems comprising mRNA comprising reference 3′ UTR Reference 1.

FIG. 18 is a graph of % GFP positive cells obtained on day 4 after nucleofecting U2OS-BFP cells with mRNAs that encoded the HiBiT-tagged gene modifying peptide (mRNAs encoding HiBiT-tagged gene modifying polypeptides). The dotted line marks the GFP % produced by using gene modifying systems comprising mRNA comprising reference 3′UTR Reference 1.

FIG. 19 is a graph of the expression of the HiBiT-tagged gene modifying polypeptide from mRNAs comprising the various 3′ UTRs at 6 hours post-nucleofection in U2OS-naïve cells (do not express BFP). The dotted line marks the expression from the mRNA comprising the reference 3′ UTR Reference 1.

FIG. 20 is a graph of expression of the HiBiT-tagged gene modifying polypeptide from mRNAs comprising the various 3′ UTRs at 24 hours post-nucleofection in Lonza's cryopreserved mouse hepatocytes (catalog number MCCP01). The dotted line marks the expression level of the gene modifying polypeptide from mRNA Reference 1 3′ UTR.

FIG. 21 is a graph of expression of the HiBiT-tagged gene modifying polypeptide from mRNAs comparing the various 3′ UTRs at 24 hours post-nucleofection in hepatocytes freshly harvested from wild-type C57BL/6 mice and normalized to the expression level of gene modifying polypeptide from an mRNA comprising Reference 1 3′UTR (dotted line).

FIG. 22 is a graph of expression of an exemplary gene modifying polypeptide (amino acid sequence given by SEQ ID NO: 8257) in U2OS-BFP cells from mRNAs containing modified poly A tails at 6 hours post-nucleofection normalized to expression of the gene modifying polypeptide expressed from an mRNA comprising the Control-80A poly A tail.

FIG. 23 is a graph of % GFP positive cells obtained after nucleofecting USOS-BFP cells with mRNAs containing modified poly A tails normalized to the % GFP positive cells of an mRNA comprising the Control-80A poly A tail.

FIG. 24A is a graph plotting gene modifying polypeptide expression level from the mRNAs comprising various modified poly A tails (data from FIG. 22) against % GFP positive cells obtained (data from FIG. 23). Two-tailed Pearson correlation was used to calculate correlation, resulting in an R2 of 0.5896. Pearson analysis of FIG. 24A includes four data points representing mRNAs that exhibited unusually low gene modifying polypeptide expression, leading to an R2 of 0.7112. FIG. 24B is a graph of the data and two-tailed Pearson analysis of FIG. 24A and excludes the four data points representing mRNAs that exhibited unusually low gene modifying polypeptide expression, leading to an R2 of 0.7112.

FIG. 25 is a graph of expression of the gene modifying polypeptide from the mRNAs at 18 hours post-nucleofection in freshly harvested primary mouse hepatocytes.

FIG. 26A is a flow chart of the experiment evaluating the expression of gene modifying polypeptide from the mRNAs over the time course of 18 hr to 48 hr post-nucleofection in primary mouse hepatocytes. FIG. 26B is a graph of eight representative expression profiles of the gene modifying polypeptide at 18 hr, 24 hr, 42 hr, and 48 hr from eight mRNAs (six with modified poly A tails, one with the Control-80A tail, and one with External-80A tail) that were administered to primary mouse hepatocytes.

FIGS. 27A-27G show the expression profiles for all mRNAs evaluated in primary mouse hepatocytes.

FIG. 28 is a ranked graph of the AUC (Area Under the Curve) of the 18 hour to 48 hour expression profile of the gene modifying polypeptide from the mRNAs comprising different modified poly A tails.

FIG. 29 is a graph of % modification for primary mouse hepatocytes nucleofected with gene modifying systems comprising mRNAs comprising various modified poly A tails, ranked by modification %.

FIG. 30 shows a graph of the protein expression time course at 2, 4, 6, 8, and 24 hours from the mRNAs equipped with test or reference UTRs, analyzed by the Hibit assay.

FIG. 31 shows a graph of the protein expression Area Under the Curve (AUC) from 2 to 24 hours calculated from FIG. 30.

### DETAILED DESCRIPTION

Various publications, articles, patents and patent applications are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive “or” and not to an exclusive “or.” For example, a condition 1 or 2 is satisfied by any one of the following: 1 is true (or present) and 2 is false (or not present), 1 is false (or not present) and 2 is true (or present), and both 1 and 2 are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 1981; 2:482, by the homology alignment algorithm of Needleman & Wunsch, J. Mol. Biol. 1970; 48:443, by the search for similarity method of Pearson & Lipman, Proc. Nat'l. Acad. Sci. USA 1988; 85:2444, by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., 1995 Supplement (Ausubel)).

Examples of algorithms that are suitable for determining percent sequence identity and sequence similarity are the BLAST and BLAST 2.0 algorithms, which are described in Altschul et al., J. Mol. Biol. 1990; 215:403-410 and Altschul et al., Nucleic Acids Res. 1997; 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 1989; 89:10915).

In addition to calculating percent sequence identity, the BLAST algorithm also performs a statistical analysis of the similarity between two sequences (see, e.g., Karlin & Altschul, Proc. Nat'l. Acad. Sci. USA 1993; 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

The term “expression cassette,” as used herein, refers to a nucleic acid construct comprising nucleic acid elements sufficient for the expression of the nucleic acid molecule of the instant invention.

A “gRNA spacer,” as used herein, refers to a portion of a nucleic acid that has complementarity to a target nucleic acid and can, together with a gRNA scaffold, target a Cas protein to the target nucleic acid.

A “gRNA scaffold,” as used herein, refers to a portion of a nucleic acid that can bind a Cas protein and can, together with a gRNA spacer, target the Cas protein to the target nucleic acid. In some embodiments, the gRNA scaffold comprises a crRNA sequence, tetraloop, and tracrRNA sequence.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

In certain embodiments, a “polypeptide” can be a “gene modifying polypeptide.” A “gene modifying polypeptide,” and “retrotransposon gene modifying polypeptide” as used herein interchangeably to refer to a polypeptide comprising a retrotransposase reverse transcriptase domain and a retrotransposase endonuclease domain, or a polypeptide comprising an amino acid sequence having at least 75%, 80%, 85%, 90%, 95%, 96%, 97%, 98%, or 99% amino acid sequence identity to said domains, which is capable of integrating a nucleic acid sequence (e.g., a sequence provided on a template nucleic acid) into a target DNA molecule (e.g., in a mammalian host cell, such as a genomic DNA molecule in the host cell). In some embodiments, the endonuclease domain is a catalytically inactive endonuclease domain. In some embodiments, the retrotransposase reverse transcriptase domain and a retrotransposase endonuclease domain are derived from the same retrotransposase. In some embodiments, the gene modifying polypeptide is capable of integrating the sequence substantially without relying on host machinery. In some embodiments, the gene modifying polypeptide integrates a sequence into a random position in a genome, and in some embodiments, the gene modifying polypeptide integrates a sequence into a specific target site. In some embodiments, a gene modifying polypeptide includes one or more domains that, collectively, facilitate 1) binding the template nucleic acid, 2) binding the target DNA molecule, and 3) facilitate integration of the at least a portion of the template nucleic acid into the target DNA. Gene modifying polypeptides include both naturally occurring polypeptides as well as engineered variants of the foregoing, e.g., having one or more amino acid substitutions to the naturally occurring sequence. Gene modifying polypeptides also include heterologous constructs, e.g., where one or more of the domains recited above are heterologous to each other, whether through a heterologous fusion (or other conjugate) of otherwise wild-type domains, as well as fusions of modified domains, e.g., by way of replacement or fusion of a heterologous sub-domain or other substituted domain. Exemplary gene modifying polypeptides, and systems comprising them and methods of using them, that can be used in the methods provided herein are described, e.g., in WO2021/178717A2, which is incorporated herein by reference, including Tables 10, 11, X, 3A, 3B, and Z1 therein. In some embodiments, a gene modifying polypeptide integrates a sequence into a gene. In some embodiments, a gene modifying polypeptide integrates a sequence into a sequence outside of a gene. A “gene modifying system,” as used herein, refers to a system comprising a gene modifying polypeptide and a template nucleic acid.

As used herein, the term “heterologous gene modifying polypeptide” refers to a polypeptide comprising a retroviral reverse transcriptase, or a polypeptide comprising an amino acid sequence having at least 75%, 80%, 85%, 90%, 95%, 96%, 97%, 98%, or 99% amino acid sequence identity to a retroviral reverse transcriptase, which is capable of integrating a nucleic acid sequence (e.g., a sequence provided on a template nucleic acid) into a target DNA molecule (e.g., in a mammalian host cell, such as a genomic DNA molecule in the host cell). In some embodiments, the heterologous gene modifying polypeptide is capable of integrating the sequence substantially without relying on host machinery. In some embodiments, the heterologous gene modifying polypeptide integrates a sequence into a random position in a genome, and in some embodiments, the heterologous gene modifying polypeptide integrates a sequence into a specific target site. In some embodiments, the sequence that is integrated comprises a deletion, substitution, or insertion relative to the target DNA molecule. In some embodiments, a heterologous gene modifying polypeptide includes one or more domains that, collectively, facilitate 1) binding the template nucleic acid, 2) binding the target DNA molecule, and 3) facilitate integration of the at least a portion of the template nucleic acid into the target DNA. Heterologous gene modifying polypeptides include both naturally occurring polypeptides as well as engineered variants of the foregoing, e.g., having one or more amino acid substitutions to the naturally occurring sequence. Heterologous gene modifying polypeptides also include heterologous constructs, e.g., where one or more of the domains recited above are heterologous to each other, whether through a heterologous fusion (or other conjugate) of otherwise wild-type domains, as well as fusions of modified domains, e.g., by way of replacement or fusion of a heterologous sub-domain or other substituted domain. Exemplary heterologous gene modifying polypeptides, and systems comprising them and methods of using them, that can be used in the methods provided herein are described, e.g., in WO2021178720, which is incorporated herein by reference with respect to heterologous gene modifying polypeptides that comprise a retroviral reverse transcriptase domain. In some embodiments, a heterologous gene modifying polypeptide integrates a sequence into a gene. In some embodiments, a heterologous gene modifying polypeptide integrates a sequence into a sequence outside of a gene.

The term “domain,” as used herein, refers to a structure of a biomolecule that contributes to a specified function of the biomolecule. A domain may comprise a contiguous region (e.g., a contiguous sequence) or distinct, non-contiguous regions (e.g., non-contiguous sequences) of a biomolecule. Examples of protein domains include, but are not limited to, an endonuclease domain, a DNA binding domain, a reverse transcription domain; an example of a domain of a nucleic acid is a regulatory domain, such as a transcription factor binding domain. In some embodiments, a domain (e.g., a Cas domain) can comprise two or more smaller domains (e.g., a DNA binding domain and an endonuclease domain).

As used herein, “first strand” and “second strand,” are used to describe the individual DNA strands of target DNA, distinguish the two DNA strands based upon which strand the reverse transcriptase domain initiates polymerization, e.g., based upon where target primed synthesis initiates. The first strand refers to the strand of the target DNA upon which the reverse transcriptase domain initiates polymerization, e.g., where target primed synthesis initiates. The second strand refers to the other strand of the target DNA. First and second strand designations do not describe the target site DNA strands in other respects; for example, in some embodiments the first and second strands are nicked by a polypeptide described herein, but the designations ‘first’ and ‘second’ strand have no bearing on the order in which such nicks occur.

The term “heterologous,” when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid, or other self-replicating vector).

The term “nucleic acid molecule,” as used herein, refers to both RNA and DNA molecules including, without limitation, cDNA, genomic DNA and mRNA, and also includes synthetic nucleic acid molecules, such as those that are chemically synthesized or recombinantly produced, such as RNA templates, as described herein. The nucleic acid molecule can be double-stranded or single-stranded, circular or linear. If single-stranded, the nucleic acid molecule can be the sense strand or the antisense strand. Unless otherwise indicated, and as an example for all sequences described herein under the general format “SEQ ID NO:” “nucleic acid comprising SEQ ID NO: 1” refers to a nucleic acid, at least a portion which has either (i) the sequence of SEQ ID NO: 1, or (ii) a sequence complimentary to SEQ ID NO: 1. The choice between the two is dictated by the context in which SEQ ID NO: 1 is used. For instance, if the nucleic acid is used as a probe, the choice between the two is dictated by the requirement that the probe be complimentary to the desired target. Nucleic acid sequences of the present disclosure may be modified chemically or biochemically or may contain non-natural or derivatized nucleotide bases, as will be readily appreciated by those of skill in the art. Such modifications include, for example, labels, methylation, substitution of one or more naturally occurring nucleotides with an analog, inter-nucleotide modifications such as uncharged linkages (for example, methyl phosphonates, phosphotriesters, phosphoramidates, carbamates, etc.), charged linkages (for example, phosphorothioates, phosphorodithioates, etc.), pendant moieties, (for example, polypeptides), intercalators (for example, acridine, psoralen, etc.), chelators, alkylators, and modified linkages (for example, alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of a molecule. Other modifications can include, for example, analogs in which the ribose ring contains a bridging moiety or other structure such as modifications found in “locked” nucleic acids. In various embodiments, the nucleic acids are in operative association with additional genetic elements, such as tissue-specific expression-control sequence(s) (e.g., tissue-specific promoters and tissue-specific microRNA recognition sequences), as well as additional elements, such as inverted repeats (e.g., inverted terminal repeats, such as elements from or derived from viruses, e.g., AAV ITRs) and tandem repeats, inverted repeats/direct repeats (e.g., transposon inverted repeats, e.g., transposon inverted repeats also containing direct repeats, e.g., inverted repeats also containing direct repeats), homology regions (segments with various degrees of homology to a target DNA), UTRs (5′, 3′, or both 5′ and 3′ UTRs), and various combinations of the foregoing. The nucleic acid elements of the systems provided by the invention can be provided in a variety of topologies, including single-stranded, double-stranded, circular, linear, linear with open ends, linear with closed ends, and particular versions of these, such as doggybone DNA (dbDNA), close-ended DNA (ceDNA).

As used herein, “insertion” of a sequence into a target site refers to the net addition of DNA sequence at the target site, e.g., where there are new nucleotides in the heterologous object sequence with no cognate positions in the unedited target site. In some embodiments, a nucleotide alignment of the primer binding site (PBS) sequence and heterologous object sequence to the target nucleic acid sequence would result in an alignment gap in the target nucleic acid sequence.

As used herein, a “deletion” generated by a heterologous object sequence in a target site refers to the net deletion of DNA sequence at the target site, e.g., where there are nucleotides in the unedited target site with no cognate positions in the heterologous object sequence. In some embodiments, a nucleotide alignment of the PBS sequence and heterologous object sequence to the target nucleic acid sequence would result in an alignment gap in the molecule comprising the PBS sequence and heterologous object sequence.

The term “mutation region,” as used herein, refers to a region in a template RNA having one or more sequence difference relative to the corresponding sequence in a target nucleic acid. The sequence difference may comprise, for example, a substitution, insertion, frameshift, or deletion.

The term “mutated” when applied to nucleic acid sequences means that nucleotides in a nucleic acid sequence are inserted, deleted, or changed compared to a reference (e.g., native) nucleic acid sequence. A single alteration may be made at a locus (a point mutation), or multiple nucleotides may be inserted, deleted, or changed at a single locus. In addition, one or more alterations may be made at any number of loci within a nucleic acid sequence. A nucleic acid sequence may be mutated by any method known in the art.

As used herein, a “gene expression unit” is a nucleic acid sequence comprising at least one regulatory nucleic acid sequence operably linked to at least one effector sequence. A first nucleic acid sequence is operably linked with a second nucleic acid sequence when the first nucleic acid sequence is placed in a functional relationship with the second nucleic acid sequence. For instance, a promoter or enhancer is operably linked to a coding sequence if the promoter or enhancer affects the transcription or expression of the coding sequence. Operably linked DNA sequences may be contiguous or non-contiguous. Where necessary to join two protein-coding regions, operably linked sequences may be in the same reading frame.

The terms “host genome” or “host cell,” as used herein, refer to a cell and/or its genome into which protein and/or genetic material has been introduced. It should be understood that such terms are intended to refer not only to the particular subject cell and/or genome, but to the progeny of such a cell and/or the genome of the progeny of such a cell. Because certain modifications may occur in succeeding generations due to either mutation or environmental influences, such progeny may not, in fact, be identical to the parent cell, but are still included within the scope of the term “host cell” as used herein. A host genome or host cell may be an isolated cell or cell line grown in culture, or genomic material isolated from such a cell or cell line or may be a host cell or host genome which composing living tissue or an organism. In some instances, a host cell may be an animal cell or a plant cell, e.g., as described herein. In certain instances, a host cell may be a mammalian cell, a human cell, avian cell, reptilian cell, bovine cell, horse cell, pig cell, goat cell, sheep cell, chicken cell, or turkey cell. In certain instances, a host cell may be a corn cell, soy cell, wheat cell, or rice cell.

As used herein, “operative association” describes a functional relationship between two nucleic acid sequences, such as a 1) promoter and 2) a heterologous object sequence, and means, in such example, the promoter and heterologous object sequence (e.g., a gene of interest) are oriented such that, under suitable conditions, the promoter drives expression of the heterologous object sequence. For instance, a template nucleic acid carrying a promoter and a heterologous object sequence may be single-stranded, e.g., either the (+) or (−) orientation. An “operative association” between the promoter and the heterologous object sequence in this template means that, regardless of whether the template nucleic acid will be transcribed in a particular state, when it is in the suitable state (e.g., is in the (+) orientation, in the presence of required catalytic factors, and NTPs, etc.), it is accurately transcribed. Operative association applies analogously to other pairs of nucleic acids, including other tissue-specific expression control sequences (such as enhancers, repressors and microRNA recognition sequences), IR/DR, ITRs, UTRs, or homology regions and heterologous object sequences or sequences encoding a retroviral RT domain.

The term “primer binding site sequence” or “PBS sequence,” as used herein, refers to a portion of a template RNA capable of binding to a region comprised in a target nucleic acid sequence. In some instances, a PBS sequence is a nucleic acid sequence comprising at least 3, 4, 5, 6, 7, or 8 bases with 100% identity to the region comprised in the target nucleic acid sequence. In some embodiments the primer region comprises at least 5, 6, 7, 8 bases with 100% identity to the region comprised in the target nucleic acid sequence. Without wishing to be bound by theory, in some embodiments when a template RNA comprises a PBS sequence and a heterologous object sequence, the PBS sequence binds to a region comprised in a target nucleic acid sequence, allowing a reverse transcriptase domain to use that region as a primer for reverse transcription, and to use the heterologous object sequence as a template for reverse transcription.

### Gene Modifying RNA Molecules and Systems Comprising the Same

Genome engineering promises tremendous therapeutic potential, including the ability to permanently address genetic diseases. Existing methods of genome engineering, however, are limited by, for example, levels of expression and/or stability of gene editing polypeptides in the host cells which are to be edited. Accordingly, a need exists for improved methods of genome engineering that account for the need for improved systems and expression constructs for the gene editing polypeptides.

The invention provides, inter alia, artificial ribonucleic acid (RNA) molecules for enhancing expression of a polypeptide, preferably a gene modifying polypeptide. The polypeptide can, for example, comprise a reverse transcriptase (RT) domain and optionally an endonuclease domain. The artificial RNA molecules can, for example, comprise 5′ and/or 3′ untranslated region (UTR) elements and/or a poly(A) tail that are specifically designed to enhance expression of the polypeptide of interest.

Thus, provided herein are artificial ribonucleic acid (RNA) molecule for enhancing expression of a polypeptide comprising a reverse transcriptase (RT) domain and optionally an endonuclease domain. The artificial RNA molecules can, for example, comprise (1) a nucleotide sequence encoding the polypeptide; (2) at least one of: (a) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269, and 8281; and/or (b) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or (3) a poly(A) tail for enhancing expression of the polypeptide, wherein the poly(A) tail comprises a nucleic acid sequence selected from the group consisting of SEQ ID NOs: 8282-8314.

In certain embodiments, the RNA molecules can, for example, comprise (1) a nucleotide sequence encoding the polypeptide; (2) at least one of: (a) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269, and 8281; and/or (b) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263.

In certain embodiments, the RNA molecules can, for example, comprise (1) a nucleotide sequence encoding the polypeptide; and (2) a poly(A) tail for enhancing expression of the polypeptide, wherein the poly(A) tail comprises a nucleic acid sequence selected from the group consisting of SEQ ID NOs: 8282-8314.

In certain embodiments, the artificial ribonucleic acid (RNA) molecules comprise (1) at least one of: (a) a 3′-untranslated region (3′UTR) element comprising a nucleic acid sequence selected from the group consisting of SEQ ID NOs: 8201-8211; and/or (b) a 5′-untranslated region (5′UTR) element comprising a nucleic acid sequence selected form the group consisting of SEQ ID NOs: 8212-8247; and/or (2) a poly(A) tail for enhancing expression of the polypeptide, wherein the poly(A) tail comprises a nucleic acid sequence selected from the group consisting of SEQ ID NOs: 8282-8314.

In certain embodiments, the artificial ribonucleic acid (RNA) molecules comprise at least one of: (a) a 3′-untranslated region (3′UTR) element comprising a nucleic acid sequence selected from the group consisting of SEQ ID NOs: 8201-8211, 8256, 8264-8269, and 8281; and/or (b) a 5′-untranslated region (5′UTR) element comprising a nucleic acid sequence selected form the group consisting of SEQ ID NOs: 8212-8247 and 8259-8263.

In certain embodiments, the artificial ribonucleic acid (RNA) molecules comprise a poly(A) tail for enhancing expression of the polypeptide, wherein the poly(A) tail comprises a nucleic acid sequence selected from the group consisting of SEQ ID NOs: 8282-8314.

In certain embodiments, the 3′UTR element comprises a nucleic acid sequence with at least 80%, 85%, 90%, 91%, 92%, 93%, 94%, 95%, 96%, 97%, 98%, 99% or 100% identity to a nucleic acid sequence selected from the group consisting of SEQ ID NOs: 8201-8211, 8256, 8264-8269, and 8281. In certain embodiments, the 3′UTR element consists of a nucleic acid sequence selected from the group consisting of SEQ ID NOs: 8201-8211, 8256, 8264-8269, and 8281. In certain embodiments, the 3′UTR element comprises a nucleic acid sequence selected from the group of SEQ ID NOs: 8256 and 8264-8269, wherein the nucleic acid sequence does not comprise the 3′ CUAG nucleotides.

In certain embodiments, the 3′UTR element of the RNA molecule comprises a nucleic acid sequence of SEQ ID NO: 8201, SEQ ID NO: 8204, SEQ ID NO: 8209, SEQ ID NO: 8211, SEQ ID NO: 8256, or SEQ ID NO: 8281. In certain embodiments, the 3′ UTR element of the RNA molecule consists of a nucleic acid sequence of SEQ ID NO: 8201, SEQ ID NO: 8204, SEQ ID NO: 8209, SEQ ID NO: 8211, SEQ ID NO: 8256, or SEQ ID NO: 8281. In certain embodiments, the 3′UTR element of the artificial RNA molecule comprises a nucleic acid sequence of SEQ ID NO: 8201, SEQ ID NO: 8204, SEQ ID NO: 8209, or SEQ ID NO: 8211. In certain embodiments, the 3′UTR element of the artificial RNA molecule consists of a nucleic acid sequence of SEQ ID NO: 8201, SEQ ID NO: 8204, SEQ ID NO: 8209, or SEQ ID NO: 8211.

In certain embodiments, the 5′UTR element comprises a nucleic acid sequence with at least 80%, 85%, 90%, 91%, 92%, 93%, 94%, 95%, 96%, 97%, 98%, 99%, or 100% identity to a nucleic acid sequence selected from the group consisting of SEQ ID NOs: 8212- 8247 and 8259-8263. In certain embodiments, the 5′UTR element consists of a nucleic acid sequence selected from the group consisting of SEQ ID NOs: 8212-8247 and 8259-8263.

In certain embodiments, the 5′UTR element of the RNA molecule comprises a nucleic acid sequence of SEQ ID NO: 8214, SEQ ID NO: 8230, SEQ ID NO: 8235, SEQ ID NO: 8236, SEQ ID NO: 8243, SEQ ID NO: 8259, or SEQ ID NO: 8263. In certain embodiments, the 5′UTR element of the RNA molecule consists of a nucleic acid sequence of SEQ ID NO: 8214, SEQ ID NO: 8230, SEQ ID NO: 8235, SEQ ID NO: 8236, SEQ ID NO: 8243, SEQ ID NO: 8259, or SEQ ID NO: 8263. In certain embodiments, the 5′UTR element of the artificial RNA molecule comprises a nucleic acid sequence of SEQ ID NO: 8214, SEQ ID NO: 8230, SEQ ID NO: 8235, SEQ ID NO: 8236, or SEQ ID NO: 8243. In certain embodiments, the 5′UTR element of the artificial RNA molecule consists of a nucleic acid sequence of SEQ ID NO: 8214, SEQ ID NO: 8230, SEQ ID NO: 8235, SEQ ID NO: 8236, or SEQ ID NO: 8243.

In certain embodiments, the poly(A) tail for enhancing expression of the polypeptide comprises a nucleic acid sequence with at least 80%, 85%, 90%, 91%, 92%, 93%, 94%, 95%, 96%, 97%, 98%, 99%, or 100% identity to the nucleic acid sequence selected from the group consisting of SEQ ID NOs: 8201-8233. In certain embodiments, the poly(A) tail for enhancing expression of the polypeptide consists of SEQ ID NOs: 8201-8233.

In certain embodiments, the poly(A) tail comprises a nucleic acid sequence of SEQ ID NO: 8213, 8218, 8225, 8226, 8228, 8230, 8231, or 8233. In certain embodiments, the poly(A) tail consists of a nucleic acid sequence of SEQ ID NO: 8213, 8218, 8225, 8226, 8228, 8230, 8231, or 8233.

In certain embodiments, the artificial RNA molecule comprises the 3′UTR element and the 5′UTR element, wherein (1) the 3′UTR element comprises SEQ ID NO: 8209 and the 5′UTR element comprises SEQ ID NO: 8236; (2) the 3′UTR element comprises SEQ ID NO: 8201 and the 5′UTR element comprises SEQ ID NO: 8236; (3) the 3′UTR element comprises SEQ ID NO: 8209 and the 5′UTR element comprises SEQ ID NO: 8214; (4) the 3′UTR element comprises SEQ ID NO: 8209 and the 5′UTR element comprises SEQ ID NO: 8243; (5) the 3′UTR element comprises SEQ ID NO: 8281 and the 5′UTR element comprises SEQ ID NO: 8259; or (6) the 3′UTR element comprises SEQ ID NO: 8281 and the 5′UTR element comprises SEQ ID NO: 8263. In certain embodiments, the artificial RNA molecule comprises the 3′UTR element and the 5′UTR element, wherein (1) the 3′UTR element consists of SEQ ID NO: 8209 and the 5′UTR element consists of SEQ ID NO: 8236; (2) the 3′UTR element consists of SEQ ID NO: 8201 and the 5′UTR element consists of SEQ ID NO: 8236; (3) the 3′UTR element consists of SEQ ID NO: 8209 and the 5′UTR element consists of SEQ ID NO: 8214; (4) the 3′UTR element consists of SEQ ID NO: 8209 and the 5′UTR element consists of SEQ ID NO: 8243; (5) the 3′UTR element consists of SEQ ID NO: 8281 and the 5′UTR element consists of SEQ ID NO: 8259; or (6) the 3′UTR element consists of SEQ ID NO: 8281 and the 5′UTR element consists of SEQ ID NO: 8263.

In certain embodiments, the artificial RNA molecule comprises the poly(A) tail, wherein the poly(A) tail comprises the nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314. In certain embodiments, the artificial RNA molecule comprises the poly(A) tail, wherein the poly(A) tail consists of the nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

In certain embodiments, the artificial RNA molecule comprises the 3′UTR element, the 5′UTR element, and the poly(A) tail, wherein (1) the 3′UTR element comprises SEQ ID NO: 8209 and the 5′UTR element comprises SEQ ID NO: 8236; (2) the 3′UTR element comprises SEQ ID NO: 8201 and the 5′UTR element comprises SEQ ID NO: 8236; (3) the 3′UTR element comprises SEQ ID NO: 8209 and the 5′UTR element comprises SEQ ID NO: 8214; (4) the 3′UTR element comprises SEQ ID NO: 8209 and the 5′UTR element comprises SEQ ID NO: 8243; (5) the 3′UTR element comprises SEQ ID NO: 8281 and the 5′UTR element comprises SEQ ID NO: 8259; or (6) the 3′UTR element comprises SEQ ID NO: 8281 and the 5′UTR element comprises SEQ ID NO: 8263; and the poly(A) tail comprises a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314. In certain embodiments, the artificial RNA molecule comprises the 3′UTR element, the 5′UTR element, and the poly(A) tail, wherein (1) the 3′UTR element comprises SEQ ID NO: 8209 and the 5′UTR element comprises SEQ ID NO: 8236; (2) the 3′UTR element comprises SEQ ID NO: 8201 and the 5′UTR element comprises SEQ ID NO: 8236; (3) the 3′UTR element comprises SEQ ID NO: 8209 and the 5′UTR element comprises SEQ ID NO: 8214; or (4) the 3′UTR element comprises SEQ ID NO: 8209 and the 5′UTR comprises SEQ ID NO: 8243; and the poly(A) tail comprises a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

In certain embodiments, the artificial RNA molecule comprises the 3′UTR element, the 5′UTR element, and the poly(A) tail, wherein (1) the 3′UTR element comprises SEQ ID NO: 8209, the 5′UTR comprises SEQ ID NO: 8236, and the poly(A) tail comprises SEQ ID NO: 8306 or SEQ ID NO: 8307; (2) the 3′UTR element comprises SEQ ID NO: 8201, the 5′UTR element comprises SEQ ID NO: 8236, and the poly(A) tail comprises SEQ ID NO: 8306 or SEQ ID NO: 8307; (3) the 3′UTR element comprises SEQ ID NO: 8209, the 5′UTR element comprises SEQ ID NO: 8214, the poly(A) tail comprises SEQ ID NO: 8306 or SEQ ID NO: 8307; (4) the 3′UTR element comprises SEQ ID NO: 8209, the 5′UTR element comprises SEQ ID NO: 8243, and the poly(A) tail comprises SEQ ID NO: 8306 or SEQ ID NO: 8307; (5) the 3′UTR element comprises SEQ ID NO: 8281, the 5′UTR element comprises SEQ ID NO: 8259, the poly(A) tail comprises SEQ ID NO: 8306 or SEQ ID NO: 8307; or (6) the 3′UTR element comprises SEQ ID NO: 8281, the 5′UTR element comprises SEQ ID NO: 8263, and the poly(A) tail comprises SEQ ID NO: 8306 or SEQ ID NO: 8307. In certain embodiments, the artificial RNA molecule comprises the 3′UTR element, the 5′UTR element, and the poly(A) tail, wherein (1) the 3′UTR element comprises SEQ ID NO: 8209, the 5′UTR comprises SEQ ID NO: 8236, and the poly(A) tail comprises SEQ ID NO: 8306 or SEQ ID NO: 8307; (2) the 3′UTR element comprises SEQ ID NO: 8201, the 5′UTR element comprises SEQ ID NO: 8236, and the poly(A) tail comprises SEQ ID NO: 8306 or SEQ ID NO: 8307; (3) the 3′UTR element comprises SEQ ID NO: 8209, the 5′UTR element comprises SEQ ID NO: 8214, the poly(A) tail comprises SEQ ID NO: 8306 or SEQ ID NO: 8307; or (4) the 3′UTR element comprises SEQ ID NO: 8209, the 5′UTR element comprises SEQ ID NO: 8243, and the poly(A) tail comprises SEQ ID NO: 8306 or SEQ ID NO: 8307.

In certain embodiments, the artificial RNA molecule comprises the 3′UTR element, the 5′UTR element, and the poly(A) tail, wherein (1) the 3′UTR element comprises SEQ ID NO: 8201, the 5′UTR element comprises SEQ ID NO: 8236, and the poly(A) tail comprises SEQ ID NO:8307; (2) the 3′UTR element comprises SEQ ID NO: 8209, the 5′UTR element comprises SEQ ID NO: 8214, and the poly(A) tail comprises SEQ ID NO:8306. In certain embodiments, the artificial RNA molecule comprises the 3′UTR element, the 5′UTR element, and the poly(A) tail, wherein (1) the 3′UTR element consists of SEQ ID NO: 8201, the 5′UTR element consists of SEQ ID NO: 8236, and the poly(A) tail consists of SEQ ID NO: 8307; (2) the 3′UTR element consists of SEQ ID NO: 8209, the 5′UTR element consists of SEQ ID NO: 8214, and the poly(A) tail consists of SEQ ID NO:8306.

Also provided is an artificial ribonucleic acid (RNA) molecule for enhancing expression of a polypeptide comprising a reverse transcriptase (RT) domain and optionally an endonuclease domain, the RNA molecule comprising (a) a nucleotide sequence encoding the polypeptide; and (b) a poly(A) tail for enhancing expression of the polypeptide, wherein the poly(A) tail consists of a nucleic acid sequence comprising a pattern of 12-18 adenosine nucleotides followed by at least two non-adenosine nucleotides, 22-28 adenosine nucleotides followed by at least two non-adenosine nucleotides, 32-38 adenosine nucleotides followed by at least two non-adenosine nucleotides, and 42-48 adenosine nucleotides.

In certain embodiments, the artificial RNA molecule consists of a nucleic acid sequence comprising a pattern of 13-17 adenosine nucleotides followed by 1-4 non-adenosine nucleotides, 23-27 adenosine nucleotides followed by 1-5 non-adenosine nucleotides, 33-37 adenosine nucleotides followed by 2-6 non-adenosine nucleotides, and 43-47 adenosine nucleotides. In certain embodiments, the artificial RNA molecule consists of a nucleic acid sequence comprising a pattern of 14-16 adenosine nucleotides followed by at least two (e.g., 2-6) non-adenosine nucleotides, 24-26 adenosine nucleotides followed by at least two (e.g., 2-6) non-adenosine nucleotides, 34-36 adenosine nucleotides followed by at least two (e.g., 2-6) non-adenosine nucleotides, and 44-46 adenosine nucleotides. In certain embodiments, the artificial ribonucleic acid (RNA) molecule consists of a nucleic acid sequence comprising a pattern of 15 adenosine nucleotides followed by at least two (e.g., 2-4) non-adenosine nucleotides, 25 adenosine nucleotides followed by at least two (e.g., 2-4) non-adenosine nucleotides, 35 adenosine nucleotides followed by at least two (e.g., 2-4) non-adenosine nucleotides, and 45 adenosine nucleotides. In certain embodiments, the artificial RNA molecule consists of a nucleic acid sequence comprising a pattern of 15 adenosine nucleotides followed by 1-4 non-adenosine nucleotides, 25 adenosine nucleotides followed by 1-5 non-adenosine nucleotides, 35 adenosine nucleotides followed by 2-6 non-adenosine nucleotides, and 45 adenosine nucleotides.

In certain embodiments, there are two to ten non-adenosine nucleotides. In certain embodiments, from 5′ to 3′ of the artificial RNA molecule, the number of non-adenosine nucleotides in each group of non-adenosine nucleotides increases. In certain embodiments, from 5′ to 3′ of the artificial RNA molecule, the number of non-adenosine nucleotides in each group of non-adenosine nucleotides is 2, 3, and 4 non-adenosine nucleotides, respectively.

In certain embodiments, the non-adenosine nucleotides are selected from a cytosine nucleotide or a uridine nucleotide.

In certain embodiments, the artificial RNA molecule comprises one or more chemically modified nucleotides.

Also provided are deoxyribonucleic acid (DNA) molecules encoding an artificial RNA molecule of the instant invention.

### Gene Modifying Polypeptides

A gene modifying polypeptide, in some embodiments, acts as a substantially autonomous protein machine capable of integrating a template nucleic acid sequence into a target DNA molecule (e.g., in a mammalian host cell, such as a genomic DNA molecule in the host cell), substantially without relying on host machinery. For example, the gene modifying polypeptide may comprise a DNA-binding domain, a reverse transcriptase domain, and an endonuclease domain. In some embodiments, the DNA-binding function may involve an RNA component that directs the protein to a DNA sequence, e.g., a gRNA spacer. In other embodiments, the gene modifying polypeptide may comprise a reverse transcriptase domain and an endonuclease domain. In some embodiments, an RNA template element is provided with a gene modifying system, wherein the RNA template element is typically heterologous to the gene modifying polypeptide element and provides an object sequence to be inserted (reverse transcribed) into the host genome. In some embodiments, the gene modifying polypeptide is capable of target primed reverse transcription. In some embodiments, the gene modifying polypeptide is capable of second-strand synthesis.

Gene modifying polypeptides suitable for use in the compositions and methods described herein include, e.g., polypeptides comprising reverse transcriptases (e.g., retroviral or retrotransposon reverse transcriptases), retrotransposases, DNA transposases, and recombinases (e.g., serine recombinases and tyrosine recombinases). Exemplary Gene Writer polypeptides, i.e., gene modifying polypeptides, and systems comprising them and methods of using them are described, e.g., in WO2020/047124 and WO2021/178720, which are incorporated by reference herein in their entirety, including the amino acid and nucleic acid sequences therein.

For example, Table 3 of WO2020/047124 is herein incorporated by reference in its entirety. In some embodiments, a gene modifying polypeptide comprises an amino acid sequence of column 8 of Table 3 of WO2020/047124, or any domain thereof (e.g., a DNA binding domain, RNA binding domain, endonuclease domain, or RT domain) or a sequence having at least 70%, 75%, 80%, 85%, 90%, 95%, 96%, 97%, 98%, or 99% identity thereto. In some embodiments, a template RNA comprises a sequence of Table 3 of WO2020/047124 (e.g., one or both of a 5′ untranslated region of column 6 and a 3′ untranslated region of column 7), or a sequence having at least 70%, 75%, 80%, 85%, 90%, 95%, 96%, 97%, 98%, or 99% identity thereto.

Exemplary gene modifying polypeptides, systems comprising the gene modifying polypeptides, and methods of using the gene modifying polypeptides are also described, e.g., in WO2021/178720, which is incorporated herein by reference with respect to retroviral RT domains, including the amino acid and nucleic acid sequences therein. The exemplary gene modifying polypeptides and retroviral RT domain sequences are described in Table 30, Table 31, and Table 44 in WO2021/178720. Accordingly, a gene modifying polypeptide described herein may comprise an amino acid sequence according to any of the Tables mentioned, or a domain thereof (e.g., a retroviral RT domain), or a functional fragment or variant of any of the foregoing, or an amino acid sequence having at least 70%, 80%, 85%, 90%, 95%, or 99% identity thereto.

Exemplary retrotransposon gene modifying polypeptides, template nucleic acids, and systems comprising the same are also described, e.g., in Tables 3A, 3B, 10, and 11 of WO2021178717A2, which are incorporated by reference herein in their entirety.

In some embodiments, the first gene modifying polypeptide is combined with a second polypeptide in a gene modifying system. In some embodiments, the second polypeptide may comprise an endonuclease domain. In some embodiments, the second polypeptide may comprise a polymerase domain, e.g., a reverse transcriptase domain. In some embodiments, the second polypeptide may comprise a DNA-dependent DNA polymerase domain. In some embodiments, the second polypeptide aids in completion of the genome edit, e.g., by contributing to second-strand synthesis or DNA repair resolution.

In some embodiments, a gene modifying polypeptide includes one or more domains that, collectively, facilitate 1) binding the template nucleic acid, 2) binding the target DNA molecule, and 3) facilitate integration of the at least a portion of the template nucleic acid into the target DNA. In some embodiments, the gene modifying polypeptide is an engineered polypeptide that comprises one or more amino acid substitutions to a corresponding naturally occurring sequence. In some embodiments, the gene modifying polypeptide comprises two or more domains that are heterologous relative to each other, e.g., through a heterologous fusion (or other conjugate) of otherwise wild-type domains, or well as fusions of modified domains, e.g., by way of replacement or fusion of a heterologous sub-domain or other substituted domain. For instance, in some embodiments, one or more of: the RT domain is heterologous to the DNA binding domain (DBD); the DBD is heterologous to the endonuclease domain; or the RT domain is heterologous to the endonuclease domain.

A functional gene modifying polypeptide can be made up of unrelated DNA binding, reverse transcription, and endonuclease domains. This modular structure allows combining of functional domains, e.g., dCas9 (DNA binding), MMLV reverse transcriptase (reverse transcription), FokI (endonuclease). In some embodiments, multiple functional domains may arise from a single protein, e.g., Cas9 or Cas9 nickase (DNA binding, endonuclease).

In some embodiments, a gene modifying polypeptide as described herein comprises a reverse transcriptase or RT domain (e.g., as described herein) that comprises a MoMLV RT sequence or variant thereof. In embodiments, the MoMLV RT sequence comprises one or more mutations selected from D200N, L603W, T330P, T306K, W313F, D524G, E562Q, D583N, P51L, S67R, E67K, T197A, H204R, E302K, F309N, L435G, N454K, H594Q, D653N, R110S, and K103L. In embodiments, the MoMLV RT sequence comprises a combination of mutations, such as D200N, L603W, and T330P, optionally further including T306K and/or W313F.

In some embodiments, the gene modifying polypeptide comprises an endonuclease domain of (e.g., as described herein) nCas9, e.g., comprising an N863A mutation (e.g., in spCas9) or a H840A mutation.

In certain embodiments, the polypeptide comprises the reverse transcriptase domain and the endonuclease domain. The endonuclease domain can, for example, be a nickase domain, such as a Cas9 domain selected from SpCas9 domain, a BlatCas9 domain, a Nme2 Cas9 domain, a PnpCas9 domain, a SauCas9 domain, a SauCas9-KKH domain, a SauriCas9 domain, a SauriCas9-KKH domain, a ScaCas9-Sc++ domain, a SpyCas9 domain, a SpyCas9-NG domain, a SpyCas9-SpRY domain, or a St1Cas9 domain. In certain embodiments, the Cas9 domain comprising an N670A mutation, an N611A mutation, an N605A mutation, an N580A mutation, an N588A mutation, an N872A mutation, an N863A mutation, an N622A mutation, or an H840A mutation.

The reverse transcriptase domain can, for example, be selected from a retrovirus transcriptase domain. In certain embodiments, the retrovirus reverse transcriptase domain is a gamma retrovirus-derived reverse transcriptase domain. The gamma retrovirus-derived reverse transcriptase domain can, for example, comprise an amino acid sequence of a reverse-transcriptase domain sequence from a family selected from AVIRE, BAEVM, FFV, FLV, FOAMV, GALV, KORV, MLVAV, MLVBM, MLVCB, MLVFF, MLVMS, PERV, SFV1, SFV3L, WMSV, and XMRV6. In certain embodiments, the gamma retrovirus-derived reverse transcriptase domain is not derived from PERV.

In certain embodiments, the reverse transcriptase domain comprises one, two, three, four, five, six or more mutations corresponding to the following mutations D200N, L603W, T330P, D524G, E562Q, D583N, P51L, S67R, T197A, H204R, E302K, F309N, W313F, L435G, N454K, H594Q, L671P, E69K, or D653N in the reverse transcriptase domain of a murine leukemia virus reverse transcriptase.

Preferably the polypeptide comprises the amino acid sequence of SEQ ID NO: 8257 or SEQ ID NO: 8371.

In some embodiments, the RT and endonuclease domains are joined by a flexible linker, e.g., comprising the amino acid sequence AEAAAKEAAAKEAAAKEAAAKALEAE AAAKEAAAKEAAAKEAAAKA (SEQ ID NO: 8275).

In some embodiments, the endonuclease domain is N-terminal relative to the RT domain. In some embodiments, the endonuclease domain is C-terminal relative to the RT domain.

In some embodiments, a gene modifying polypeptide is capable of producing a substitution into the target site of at least 1, 2, 3, 4, 5, 6, 7, 8, 9, 10, 15, 20, 25, 30, 35, 40, 45, 50, 60, 70, 80, 90, or 100 or more nucleotides. In some embodiments, the substitution is a transition mutation. In some embodiments, the substitution is a transversion mutation. In some embodiments, the substitution converts an adenine to a thymine, an adenine to a guanine, an adenine to a cytosine, a guanine to a thymine, a guanine to a cytosine, a guanine to an adenine, a thymine to a cytosine, a thymine to an adenine, a thymine to a guanine, a cytosine to an adenine, a cytosine to a guanine, or a cytosine to a thymine.

In some embodiments, an insertion, deletion, substitution, or combination thereof, increases or decreases expression (e.g., transcription or translation) of a gene. In some embodiments, an insertion, deletion, substitution, or combination thereof, increases or decreases expression (e.g., transcription or translation) of a gene by altering, adding, or deleting sequences in a promoter or enhancer, e.g., sequences that bind transcription factors. In some embodiments, an insertion, deletion, substitution, or combination thereof alters translation of a gene (e.g., alters an amino acid sequence), inserts or deletes a start or stop codon, alters or fixes the translation frame of a gene. In some embodiments, an insertion, deletion, substitution, or combination thereof alters splicing of a gene, e.g., by inserting, deleting, or altering a splice acceptor or donor site. In some embodiments, an insertion, deletion, substitution, or combination thereof alters the transcript or protein half-life. In some embodiments, an insertion, deletion, substitution, or combination thereof alters protein localization in the cell (e.g., from the cytoplasm to a mitochondria, from the cytoplasm into the extracellular space (e.g., adds a secretion tag)). In some embodiments, an insertion, deletion, substitution, or combination thereof alters (e.g., improves) protein folding (e.g., to prevent accumulation of misfolded proteins). In some embodiments, an insertion, deletion, substitution, or combination thereof, alters, increases, decreases the activity of a gene, e.g., a protein encoded by the gene.

Retargeting (e.g., of a gene modifying polypeptide or nucleic acid molecule, or of a system as described herein) generally comprises: (i) directing the polypeptide to bind and cleave at the target site; and/or (ii) designing the template RNA to have complementarity to the target sequence. In some embodiments, the template RNA has complementarity to the target sequence 5′ of the first-strand nick, e.g., such that the 3′ end of the template RNA anneals and the 5′ end of the target site serves as the primer, e.g., for target-primed reverse transcription (TPRT). In some embodiments, the endonuclease domain of the polypeptide and the 5′ end of the RNA template are also modified as described.

In some embodiments, a gene modifying polypeptide comprises a modification to a DNA-binding domain, e.g., relative to the wild-type polypeptide. In some embodiments, the DNA-binding domain comprises an addition, deletion, replacement, or modification to the amino acid sequence of the original DNA-binding domain. In some embodiments, the DNA-binding domain is modified to include a heterologous functional domain that binds specifically to a target nucleic acid (e.g., DNA) sequence of interest. In some embodiments, the functional domain replaces at least a portion (e.g., the entirety of) the prior DNA-binding domain of the polypeptide. In some embodiments, the functional domain comprises a zinc finger (e.g., a zinc finger that specifically binds to the target nucleic acid (e.g., DNA) sequence of interest). In some embodiments, the functional domain comprises a Cas domain (e.g., a Cas domain that specifically binds to the target nucleic acid (e.g., DNA) sequence of interest). In embodiments, the Cas domain comprises a Cas9 or a mutant or variant thereof (e.g., as described herein, see, e.g., SEQ ID NOs: 8331-8369). In embodiments, the Cas domain is associated with a guide RNA (gRNA), e.g., as described herein. In embodiments, the Cas domain is directed to a target nucleic acid (e.g., DNA) sequence of interest by the gRNA. In embodiments, the Cas domain is encoded in the same nucleic acid (e.g., RNA) molecule as the gRNA. In embodiments, the Cas domain is encoded in a different nucleic acid (e.g., RNA) molecule from the gRNA.

In some embodiments, a gene modifying polypeptide comprises a modification to an endonuclease domain, e.g., relative to the wild-type polypeptide. In some embodiments, the endonuclease domain comprises an addition, deletion, replacement, or modification to the amino acid sequence of the original endonuclease domain. In some embodiments, the endonuclease domain is modified to include a heterologous functional domain that binds specifically to and/or induces endonuclease cleavage of a target nucleic acid (e.g., DNA) sequence of interest. In some embodiments, the endonuclease domain comprises a zinc finger. In some embodiments, the endonuclease domain comprises a Cas domain (e.g., a Cas9 or a mutant or variant thereof). In embodiments, the endonuclease domain comprising the Cas domain is associated with a guide RNA (gRNA), e.g., as described herein. In some embodiments, the endonuclease domain is modified to include a functional domain that does not target a specific target nucleic acid (e.g., DNA) sequence. In embodiments, the endonuclease domain comprises a FokI domain.

In some embodiments, the reverse transcriptase (RT) domain exhibits enhanced stringency of target-primed reverse transcription (TPRT) initiation, e.g., relative to an endogenous RT domain. In some embodiments, the RT domain initiates TPRT when the 3 nucleotides (nt) in the target site immediately upstream of the first strand nick, e.g., the genomic DNA priming the RNA template, have at least 66% or 100% complementarity to the 3 nt of homology in the RNA template. In some embodiments, the RT domain initiates TPRT when there are less than 5 nt mismatched (e.g., less than 1, 2, 3, 4, or 5 nt mismatched) between the template RNA homology and the target DNA priming reverse transcription. In some embodiments, the RT domain is modified such that the stringency for mismatches in priming the TPRT reaction is increased, e.g., wherein the RT domain does not tolerate any mismatches or tolerates fewer mismatches in the priming region relative to a wild-type (e.g., unmodified) RT domain.

In some embodiments, the RT domain comprises a HIV-1 RT domain. In embodiments, the HIV-1 RT domain initiates lower levels of synthesis even with three nucleotide mismatches relative to an alternative RT domain (e.g., as described by Jamburuthugoda and Eickbush J Mol Biol 407(5):661-672 (2011); incorporated herein by reference in its entirety). In some embodiments, the RT domain forms a dimer (e.g., a heterodimer or homodimer). In some embodiments, the RT domain is monomeric. In some embodiments, an RT domain, naturally functions as a monomer or as a dimer (e.g., heterodimer or homodimer). In some embodiments, an RT domain naturally functions as a monomer, e.g., is derived from a virus wherein it functions as a monomer. In embodiments, the RT domain is selected from an RT domain from murine leukemia virus (MLV; sometimes referred to as MoMLV) (e.g., P03355), porcine endogenous retrovirus (PERV) (e.g., UniProt Q4VFZ2), mouse mammary tumor virus (MMTV) (e.g., UniProt P03365), Avian reticuloendotheliosis virus (AVIRE) (e.g., UniProtKB accession: P03360); Feline leukemia virus (FLV or FeLV) (e.g., e.g., UniProtKB accession: P10273); Mason-Pfizer monkey virus (MPMV) (e.g., UniProt P07572), bovine leukemia virus (BLV) (e.g., UniProt P03361), human T-cell leukemia virus-1 (HTLV-1) (e.g., UniProt P03362), human foamy virus (HFV) (e.g., UniProt P14350), simian foamy virus (SFV) (e.g., SFV3L) (e.g., UniProt P23074 or P27401), or bovine foamy/syncytial virus (BFV/BSV) (e.g., UniProt 041894), or a functional fragment or variant thereof (e.g., an amino acid sequence having at least 70%, 80%, 90%, 95%, or 99% identity thereto). In some embodiments, an RT domain is dimeric in its natural functioning. In some embodiments, the RT domain is derived from a virus wherein it functions as a dimer. In embodiments, the RT domain is selected from an RT domain from avian sarcoma/leukemia virus (ASLV) (e.g., UniProt A0A142BKH1), Rous sarcoma virus (RSV) (e.g., UniProt P03354), avian myeloblastosis virus (AMV) (e.g., UniProt Q83133), human immunodeficiency virus type I (HIV-1) (e.g., UniProt P03369), human immunodeficiency virus type II (HIV-2) (e.g., UniProt P15833), simian immunodeficiency virus (SIV) (e.g., UniProt P05896), bovine immunodeficiency virus (BIV) (e.g., UniProt P19560), equine infectious anemia virus (EIAV) (e.g., UniProt P03371), or feline immunodeficiency virus (FIV) (e.g., UniProt P16088) (Herschhorn and Hizi Cell Mol Life Sci 67 (16): 2717-2747 (2010)), or a functional fragment or variant thereof (e.g., an amino acid sequence having at least 70%, 80%, 90%, 95%, or 99% identity thereto). Naturally heterodimeric RT domains may, in some embodiments, also be functional as homodimers. In some embodiments, dimeric RT domains are expressed as fusion proteins, e.g., as homodimeric fusion proteins or heterodimeric fusion proteins. In some embodiments, the RT function of the system is fulfilled by multiple RT domains (e.g., as described herein). In further embodiments, the multiple RT domains are fused or separate, e.g., may be on the same polypeptide or on different polypeptides.

In some embodiments, a gene modifying polypeptide possesses the function of DNA target site cleavage via an endonuclease domain. In some embodiments, a gene modifying polypeptide comprises a DNA binding domain, e.g., for binding to a target nucleic acid. In some embodiments, a domain (e.g., a Cas domain) of the gene modifying polypeptide comprises two or more smaller domains, e.g., a DNA binding domain and an endonuclease domain. It is understood that when a DNA binding domain (e.g., a Cas domain) is said to bind to a target nucleic acid sequence, in some embodiments, the binding is mediated by a gRNA.

In some embodiments, a domain has two functions. For example, in some embodiments, the endonuclease domain is also a DNA-binding domain. In some embodiments, the endonuclease domain is also a template nucleic acid (e.g., template RNA) binding domain. For example, in some embodiments, a polypeptide comprises a CRISPR-associated endonuclease domain that binds a template RNA comprising a gRNA, binds a target DNA sequence (e.g., with complementarity to a portion of the gRNA), and cuts the target DNA sequence. In some embodiments, an endonuclease domain or endonuclease/DNA-binding domain from a heterologous source can be used or can be modified (e.g., by insertion, deletion, or substitution of one or more residues) in a gene modifying system described herein.

In some embodiments, the endonuclease domain has nickase activity that nicks the target site DNA of the first strand, e.g., in some embodiments, the endonuclease domain cuts the genomic DNA of the target site near to the site of alteration on the strand that will be extended by the writing domain. In some embodiments, the endonuclease domain has nickase activity that nicks the target site DNA of the first strand and does not nick the target site DNA of the second strand. For example, when a polypeptide comprises a CRISPR-associated endonuclease domain having nickase activity, in some embodiments, said CRISPR-associated endonuclease domain nicks the target site DNA strand containing the PAM site (e.g., and does not nick the target site DNA strand that does not contain the PAM site). As a further example, when a polypeptide comprises a CRISPR-associated endonuclease domain having nickase activity, in some embodiments, said CRISPR-associated endonuclease domain nicks the target site DNA strand not containing the PAM site (e.g., and does not nick the target site DNA strand that contains the PAM site).

In some embodiments, the polypeptide comprises a single domain having endonuclease activity (e.g., a single endonuclease domain) and said domain nicks both the first strand and the second strand. For example, in such an embodiment the endonuclease domain may be a CRISPR-associated endonuclease domain, and the template nucleic acid (e.g., template RNA) comprises a gRNA spacer that directs nicking of the first strand and an additional gRNA spacer that directs nicking of the second strand. In some embodiments, the polypeptide comprises a plurality of domains having endonuclease activity, and a first endonuclease domain nicks the first strand and a second endonuclease domain nicks the second strand (optionally, the first endonuclease domain does not (e.g., cannot) nick the second strand and the second endonuclease domain does not (e.g., cannot) nick the first strand).

In some embodiments, a gene modifying polypeptide described herein comprises a Cas domain. In some embodiments, the Cas domain can direct the gene modifying polypeptide to a target site specified by a gRNA spacer, thereby modifying a target nucleic acid sequence in “cis.” In some embodiments, a gene modifying polypeptide is fused to a Cas domain. In some embodiments, a gene modifying polypeptide comprises a CRISPR/Cas domain (also referred to herein as a CRISPR-associated protein). In some embodiments, a CRISPR/Cas domain comprises a protein involved in the clustered regulatory interspaced short palindromic repeat (CRISPR) system, e.g., a Cas protein, and optionally binds a guide RNA, e.g., single guide RNA (sgRNA).

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a DNA-binding domain or endonuclease domain includes a sequence targeting polypeptide, such as a Cas protein, e.g., Cas9. In certain embodiments a Cas protein, e.g., a Cas9 protein, may be obtained from a bacteria or archaea or synthesized using known methods. Additional description of CRISPR systems can be found in WO2021/178898, incorporated herein by reference in its entirety.

### Sequences of Exemplary Cas9-Linker-RT Fusions

In some embodiments, a gene modifying polypeptide (e.g., a gene modifying polypeptide that is part of a system described herein) comprises an amino acid sequence of any one of SEQ ID NOs: 1-7743, or an amino acid sequence having at least 70%, 75%, 80%, 85%, 90%, 95%, or 99% identity thereto. In some embodiments, a gene modifying polypeptide comprises an amino acid sequence of any one of SEQ ID NOs: 1-7743, or an amino acid sequence having at least 80% identity thereto. In some embodiments, a gene modifying polypeptide comprises an amino acid sequence of any one of SEQ ID NOs: 1-7743, or an amino acid sequence having at least 90% identity thereto. In some embodiments, a gene modifying polypeptide comprises an amino acid sequence of any one of SEQ ID NOS: 1-7743, or an amino acid sequence having at least 95% identity thereto. In some embodiments, a gene modifying polypeptide comprises an amino acid sequence of any one of SEQ ID NOs: 1-7743, or an amino acid sequence having at least 99% identity thereto. In some embodiments, a gene modifying polypeptide comprises an amino acid sequence of any one of SEQ ID NOs: 1-7743. In some embodiments, a gene modifying polypeptide comprises an amino acid sequence of any one of SEQ ID NOs: 6001-7743, or an amino acid sequence having at least 70%, 75%, 80%, 85%, 90%, 95%, or 99% identity thereto. In some embodiments, a gene modifying polypeptide comprises an amino acid sequence of any one of SEQ ID NOs: 4501-4541, or an amino acid sequence having at least 70%, 75%, 80%, 85%, 90%, 95%, or 99% identity thereto.

In some embodiments, a gene modifying polypeptide comprises an amino acid sequence as listed in Table T1, or an amino acid sequence having at least 70%, 75%, 80%, 85%, 90%, 95%, or 99% identity thereto. In some embodiments, a gene modifying polypeptide comprises a linker comprising a linker sequence as listed in Table T1, or an amino acid sequence having at least 70%, 75%, 80%, 85%, 90%, 95%, or 99% identity thereto. In some embodiments, a gene modifying polypeptide comprises an RT domain comprising an RT domain sequence as listed in Table T1, or an amino acid sequence having at least 70%, 75%, 80%, 85%, 90%, 95%, or 99% identity thereto. In some embodiments, a gene modifying polypeptide comprises: (i) a linker comprising a linker sequence as listed in a row of Table T1, or an amino acid sequence having at least 70%, 75%, 80%, 85%, 90%, 95%, or 99% identity thereto; and (ii) an RT domain comprising an RT domain sequence as listed in the same row of Table T1, or an amino acid sequence having at least 70%, 75%, 80%, 85%, 90%, 95%, or 99% identity thereto.

TABLE T1
Selection of exemplary gene modifying polypeptides

| SEQ ID | | SEQ ID | |
|---|---|---|---|
| NO: for Full | | NO: | |
| Polypeptide | | of | |
| Sequence | Linker Sequence | linker | RT name |
| 1372 | AEAAAKEAAAKEAAAKEAAAKALEAE | 8137 | AVIRE_P03360_3mutA |
| | AAAKEAAAKEAAAKEAAAKA | | |
| 1197 | AEAAAKEAAAKEAAAKEAAAKALEAE | 8138 | FLV_P10273_3mutA |
| | AAAKEAAAKEAAAKEAAAKA | | |
| 2784 | AEAAAKEAAAKEAAAKEAAAKALEAE | 8139 | MLVMS_P03355_3mutA_ |
| | AAAKEAAAKEAAAKEAAAKA | | WS |
| 647 | AEAAAKEAAAKEAAAKEAAAKALEAE | 8140 | SFV3L_P27401_2mutA |
| | AAAKEAAAKEAAAKEAAAKA | | |

In some embodiments, a gene modifying polypeptide comprises an amino acid sequence as listed in Table T2, or an amino acid sequence having at least 70%, 75%, 80%, 85%, 90%, 95%, or 99% identity thereto. In some embodiments, a gene modifying polypeptide comprises a linker comprising a linker sequence as listed in Table T2, or an amino acid sequence having at least 70%, 75%, 80%, 85%, 90%, 95%, or 99% identity thereto. In some embodiments, a gene modifying polypeptide comprises an RT domain comprising an RT domain sequence as listed in Table T2, or an amino acid sequence having at least 70%, 75%, 80%, 85%, 90%, 95%, or 99% identity thereto. In some embodiments, a gene modifying polypeptide comprises: (i) a linker comprising a linker sequence as listed in a row of Table T2, or an amino acid sequence having at least 70%, 75%, 80%, 85%, 90%, 95%, or 99% identity thereto; and (ii) an RT domain comprising an RT domain sequence as listed in the same row of Table T2, or an amino acid sequence having at least 70%, 75%, 80%, 85%, 90%, 95%, or 99% identity thereto.

TABLE T2
Selection of exemplary gene modifying polypeptides

| SEQ ID | | | |
|---|---|---|---|
| NO: | | SEQ | |
| for Full | | ID NO: | |
| Polypeptide | | of | |
| Sequence | Linker Sequence | linker | RT name |
| 2311 | GGGGSGGGGSGGGGSGGGGS | 8141 | MLVCB_P08361_3mutA |
| 1373 | GGGGSGGGGSGGGGSGGGGSGGGGSGGGG | 8142 | AVIRE_P03360_3mutA |
| | S | | |
| 2644 | GGGGSGGGGSGGGGSGGGGSGGGGSGGGG | 8143 | MLVMS_P03355_PLV919 |
| | S | | |
| 2304 | GSSGSSGSSGSSGSSGSS | 8144 | MLVCB_P08361_3mutA |
| 2325 | EAAAKEAAAKEAAAKEAAAK | 8145 | MLVCB_P08361_3mutA |
| 2322 | EAAAKEAAAKEAAAKEAAAKEAAAKEAA | 8146 | MLVCB_P08361_3mutA |
| | AK | | |
| 2187 | PAPAPAPAPAP | 8147 | MLVBM_Q7SVK7_3mut |
| 2309 | PAPAPAPAPAPAP | 8148 | MLVCB_P08361_3mutA |
| 2534 | PAPAPAPAPAPAP | 8149 | MLVFF_P26809_3mutA |
| 2797 | PAPAPAPAPAPAP | 8150 | MLVMS_P03355_3mutA_WS |
| 3084 | PAPAPAPAPAPAP | 8151 | MLVMS_P03355_3mutA_WS |
| 2868 | PAPAPAPAPAPAP | 8152 | MLVMS_P03355_PLV919 |
| 126 | EAAAKGGG | 8153 | PERV_Q4VFZ2_3mut |
| 306 | EAAAKGGG | 8154 | PERV_Q4VFZ2_3mut |
| 1410 | PAPGGG | 8155 | AVIRE_P03360_3mutA |
| 804 | GGGGSSGGS | 8156 | WMSV_P03359_3mut |
| 1937 | GGGGGSEAAAK | 8157 | BAEVM_P10272_3mutA |
| 2721 | GGGEAAAKGGS | 8158 | MLVMS_P03355_3mut |
| 3018 | GGGEAAAKGGS | 8159 | MLVMS_P03355_3mut |
| 1018 | GGGEAAAKGGS | 8160 | XMRV6_A1Z651_3mutA |
| 2317 | GGSGGGPAP | 8161 | MLVCB_P08361_3mutA |
| 2649 | PAPGGSGGG | 8162 | MLVMS_P03355_PLV919 |
| 2878 | PAPGGSGGG | 8163 | MLVMS_P03355_PLV919 |
| 912 | GGSEAAAKPAP | 8164 | WMSV_P03359_3mutA |
| 2338 | GGSPAPEAAAK | 8165 | MLVCB_P08361_3mutA |
| 2527 | GGSPAPEAAAK | 8166 | MLVFF_P26809_3mutA |
| 141 | EAAAKGGSPAP | 8167 | PERV_Q4VFZ2_3mut |
| 341 | EAAAKGGSPAP | 8168 | PERV_Q4VFZ2_3mut |
| 2315 | EAAAKPAPGGS | 8169 | MLVCB_P08361_3mutA |
| 3080 | EAAAKPAPGGS | 8170 | MLVMS_P03355_3mutA_WS |
| 2688 | GGGGSSEAAAK | 8171 | MLVMS_P03355_PLV919 |
| 2885 | GGGGSSEAAAK | 8172 | MLVMS_P03355_PLV919 |
| 2810 | GSSGGGEAAAK | 8173 | MLVMS_P03355_3mutA_WS |
| 3057 | GSSGGGEAAAK | 8174 | MLVMS_P03355_3mutA_WS |
| 1861 | GSSEAAAKGGG | 8175 | MLVAV_P03356_3mutA |
| 3056 | GSSGGGPAP | 8176 | MLVMS_P03355_3mutA_WS |
| 1038 | GSSPAPGGG | 8177 | XMRV6_A1Z651_3mutA |
| 2308 | PAPGGGGSS | 8178 | MLVCB_P08361_3mutA |
| 1672 | GGGEAAAKPAP | 8179 | KORV_Q9TTC1-Pro_3mutA |
| 2526 | GGGEAAAKPAP | 8180 | MLVFF_P26809_3mutA |
| 1938 | GGGPAPEAAAK | 8181 | BAEVM_P10272_3mutA |
| 2641 | GSSEAAAKPAP | 8182 | MLVMS_P03355_PLV919 |
| 2891 | GSSEAAAKPAP | 8183 | MLVMS_P03355_PLV919 |
| 1225 | GSSPAPEAAAK | 8184 | FLV_P10273_3mutA |
| 2839 | GSSPAPEAAAK | 8185 | MLVMS_P03355_3mutA_WS |
| 3127 | GSSPAPEAAAK | 8186 | MLVMS_P03355_3mutA_WS |
| 2798 | PAPGSSEAAAK | 8187 | MLVMS_P03355_3mutA_WS |
| 3091 | PAPGSSEAAAK | 8188 | MLVMS_P03355_3mutA_WS |
| 1372 | AEAAAKEAAAKEAAAKEAAAKALEAEAA | 8189 | AVIRE_P03360_3mutA |
| | AKEAAAKEAAAKEAAAKA | | |
| 1197 | AEAAAKEAAAKEAAAKEAAAKALEAEAA | 8190 | FLV_P10273_3mutA |
| | AKEAAAKEAAAKEAAAKA | | |
| 2611 | AEAAAKEAAAKEAAAKEAAAKALEAEAA | 8191 | MLVMS_P03355_PLV919 |
| | AKEAAAKEAAAKEAAAKA | | |
| 2784 | AEAAAKEAAAKEAAAKEAAAKALEAEAA | 8192 | MLVMS_P03355_3mutA_WS |
| | AKEAAAKEAAAKEAAAKA | | |
| 480 | AEAAAKEAAAKEAAAKEAAAKALEAEAA | 8193 | SFV1_P23074_2mutA |
| | AKEAAAKEAAAKEAAAKA | | |
| 647 | AEAAAKEAAAKEAAAKEAAAKALEAEAA | 8194 | SFV3L_P27401_2mutA |
| | AKEAAAKEAAAKEAAAKA | | |
| 1006 | AEAAAKEAAAKEAAAKEAAAKALEAEAA | 8195 | XMRV6_A1Z651_3mutA |
| | AKEAAAKEAAAKEAAAKA | | |
| 2518 | SGSETPGTSESATPES | 8196 | MLVFF_P26809_3mutA |

### Subsequences of Exemplary Gene Modifying Polypeptides

In some embodiments, the gene modifying polypeptide comprises, in N-terminal to C-terminal order, one or more (e.g., 1, 2, 3, 4, 5, or all 6) of an N-terminal methionine residue, a first nuclear localization signal (NLS), a DNA binding domain, a linker, an RT domain, and/or a second NLS. In some embodiments, a gene modifying polypeptide comprises, in N-terminal to C-terminal order, a NLS (e.g., a first NLS), a DNA binding domain, a linker, and an RT domain, wherein the linker and RT domain are the linker and RT domain of a gene modifying polypeptide of any one of SEQ ID NOs: 1-7743, or an amino acid sequence having at least 70%, 75%, 80%, 85%, 90%, 95%, or 99% identity to said linker and RT domain. In some embodiments, a gene modifying polypeptide comprises, in N-terminal to C-terminal order, a DNA binding domain, a linker, an RT domain, and an NLS (e.g., a second NLS) wherein the linker and RT domain are the linker and RT domain of a gene modifying polypeptide of any one of SEQ ID NOs: 1-7743, or an amino acid sequence having at least 70%, 75%, 80%, 85%, 90%, 95%, or 99% identity to said linker and RT domain. In some embodiments, a gene modifying polypeptide comprises, in N-terminal to C-terminal order, a first NLS, a DNA binding domain, a linker, an RT domain, and a second NLS, wherein the linker and RT domain are the linker and RT domain of a gene modifying polypeptide of any one of SEQ ID NOs: 1-7743, or an amino acid sequence having at least 70%, 75%, 80%, 85%, 90%, 95%, or 99% identity to said linker and RT domain. In some embodiments, the gene modifying polypeptide further comprises an N-terminal methionine residue.

In some embodiments, the gene modifying polypeptide comprises, in N-terminal to C-terminal order, one or more (e.g., 1, 2, 3, 4, 5, or all 6) of an N-terminal methionine residue, a first nuclear localization signal (NLS) (e.g., of a gene modifying polypeptide of any one of SEQ ID NOs: 1-7743 and/or as listed in any of Tables T1 or T2, or an amino acid sequence having at least 70%, 75%, 80%, 85%, 90%, 95%, or 99% identity thereto), a DNA binding domain (e.g., a Cas domain, e.g., a SpyCas9 domain, e.g., as listed in SEQ ID NOS: 8331-8369, or an amino acid sequence having at least 70%, 75%, 80%, 85%, 90%, 95%, or 99% identity thereto; or a DNA binding domain of a gene modifying polypeptide of any one of SEQ ID NOs: 1-7743 and/or as listed in any of Tables T1 or T2, or an amino acid sequence having at least 70%, 75%, 80%, 85%, 90%, 95%, or 99% identity thereto), a linker (e.g., of a gene modifying polypeptide of any one of SEQ ID NOs: 1-7743 and/or as listed in any of Tables T1 or T2, or an amino acid sequence having at least 70%, 75%, 80%, 85%, 90%, 95%, or 99% identity thereto), an RT domain (e.g., of a gene modifying polypeptide of any one of SEQ ID NOs: 1-7743 and/or as listed in any of Tables T1 or T2, or an amino acid sequence having at least 70%, 75%, 80%, 85%, 90%, 95%, or 99% identity thereto), and a second NLS (e.g., of a gene modifying polypeptide of any one of SEQ ID NOs: 1-7743 and/or as listed in any of Tables T1 or T2, or an amino acid sequence having at least 70%, 75%, 80%, 85%, 90%, 95%, or 99% identity thereto). In some embodiments, the gene modifying polypeptide further comprises (e.g., C-terminal to the second NLS) a T2A sequence and/or a puromycin sequence (e.g., of a gene modifying polypeptide of any one of SEQ ID NOs: 1-7743 and/or as listed in any of Tables T1 or T2, or an amino acid sequence having at least 70%, 75%, 80%, 85%, 90%, 95%, or 99% identity thereto). In some embodiments, a nucleic acid encoding a gene modifying polypeptide (e.g., as described herein) encodes a T2A sequence, e.g., wherein the T2A sequence is situated between a region encoding the gene modifying polypeptide and a second region, wherein the second region optionally encodes a selectable marker, e.g., puromycin.

In certain embodiments, the first NLS comprises a first NLS sequence of a gene modifying polypeptide having an amino acid sequence of any one of SEQ ID NOs: 1-7743, or an amino acid sequence having at least 70%, 75%, 80%, 85%, 90%, 95%, or 99% identity thereto. In certain embodiments, the first NLS comprises a first NLS sequence of a gene modifying polypeptide as listed in any of Tables T1 or T2, or an amino acid sequence having at least 70%, 75%, 80%, 85%, 90%, 95%, or 99% identity thereto. In certain embodiments, the first NLS sequence comprises a C-myc NLS. In certain embodiments, the first NLS comprises the amino acid sequence PAAKRVKLD (SEQ ID NO: 8277), or an amino acid sequence having at least 70%, 75%, 80%, 85%, 90%, 95%, or 99% identity thereto.

In certain embodiments, the gene modifying polypeptide further comprises a spacer sequence between the first NLS and the DNA binding domain. In certain embodiments, the spacer sequence between the first NLS and the DNA binding domain comprises 1, 2, 3, 4, 5, 6, 7, 8, 9, or 10 amino acids. In certain embodiments, the spacer sequence between the first NLS and the DNA binding domain comprises the amino acid sequence GG.

In certain embodiments, the DNA binding domain comprises a DNA binding domain of a gene modifying polypeptide of any one of SEQ ID NOs: 1-7743, or an amino acid sequence having at least 70%, 75%, 80%, 85%, 90%, 95%, or 99% identity thereto. In certain embodiments, the DNA binding domain comprises a DNA binding domain of a gene modifying polypeptide as listed in any of Tables T1 or T2, or an amino acid sequence having at least 70%, 75%, 80%, 85%, 90%, 95%, or 99% identity thereto. In certain embodiments, the DNA binding domain comprises a Cas domain (e.g., as listed in SEQ ID NOs: 8331-8369). In certain embodiments, the DNA binding domain comprises the amino acid sequence of a Spy Cas9 polypeptide (e.g., as listed in SEQ ID NOs: 8331-8369, e.g., a Cas9 N863A polypeptide), or an amino acid sequence having at least 70%, 75%, 80%, 85%, 90%, 95%, or 99% identity thereto. In certain embodiments, the DNA binding domain comprises the amino acid sequence: DKKYSIGLDIGTNSVGWAVITDEYKVPSKKFKVLGNTDRHSIKKNLIGALLFDSGETAEATR LKRTARRRYTRRKNRICYLQEIFSNEMAKVDDSFFHRLEESELVEEDKKHERHPIFGNIVDE VAYHEKYPTIYHLRKKLVDSTDKADLRLIYLALAHMIKFRGHFLIEGDLNPDNSDVDKLFIQ LVQTYNQLFEENPINASGVDAKAILSARLSKSRRLENLIAQLPGEKKNGLFGNLIALSLGLT PNFKSNFDLAEDAKLQLSKDTYDDDLDNLLAQIGDQYADLFLAAKNLSDAILLSDILRVNTE ITKAPLSASMIKRYDEHHQDLTLLKALVROQLPEKYKEIFFDQSKNGYAGYIDGGASQEEFY KFIKPILEKMDGTEELLVKLNREDLLRKORTFDNGSIPHQIHLGELHAILRRQEDFYPFLKD NREKIEKILTFRIPYYVGPLARGNSRFAWMTRKSEETITPWNFEEVVDKGASAQSFIERMTN FDKNLPNEKVLPKHSLLYEYFTVYNELTKVKYVTEGMRKPAFLSGEQKKAIVDLLEKTNRKV TVKQLKEDYFKKIECFDSVEISGVEDRENASLGTYHDLLKIIKDKDELDNEENEDILEDIVL TLTLFEDREMIEERLKTYAHLFDDKVMKOLKRRRYTGWGRLSRKLINGIRDKQSGKTILDFL KSDGFANRNEMQLIHDDSLTFKEDIQKAQVSGOGDSLHEHIANLAGSPAIKKGILQTVKVVD ELVKVMGRHKPENIVIEMARENQTTQKGQKNSRERMKRIEEGIKELGSQILKEHPVENTQLQ NEKLYLYYLONGRDMYVDQELDINRLSDYDVDHIVPQSFLKDDSIDNKVLTRSDKARGKSDN VPSEEVVKKMKNYWRQLLNAKLITQRKFDNLTKAERGGLSELDKAGFIKRQLVETRQITKHV AQILDSRMNTKYDENDKLIREVKVITLKSKLVSDFRKDFQFYKVREINNYHHAHDAYLNAVV GTALIKKYPKLESEFVYGDYKVYDVRKMIAKSEQEIGKATAKYFFYSNIMNFFKTEITLANG EIRKRPLIETNGETGEIVWDKGRDFATVRKVLSMPQVNIVKKTEVQTGGFSKESILPKRNSD KLIARKKDWDPKKYGGFDSPTVAYSVLVVAKVEKGKSKKLKSVKELLGITIMERSSFEKNPI DFLEAKGYKEVKKDLIIKLPKYSLFELENGRKRMLASAGELOKGNELALPSKYVNFLYLASH YEKLKGSPEDNEQKQLFVEQHKHYLDEIIEQISEFSKRVILADANLDKVLSAYNKHRDKPIR EQAENIIHLFTLTNLGAPAAFKYFDTTIDRKRYTSTKEVLDATLIHQSITGLYETRIDLSQL GGD (SEQ ID NO: 8197), or an amino acid sequence having at least 70%, 75%, 80%, 85%, 90%, 95%, or 99% identity thereto.

In certain embodiments, the gene modifying polypeptide further comprises a spacer sequence between the DNA binding domain and the linker. In certain embodiments, the spacer sequence between the DNA binding domain and the linker comprises 1, 2, 3, 4, 5, 6, 7, 8, 9, or 10 amino acids. In certain embodiments, the spacer sequence between the DNA binding domain and the linker comprises the amino acid sequence GG.

In certain embodiments, the linker comprises a linker sequence of a gene modifying polypeptide of any one of SEQ ID NOs: 1-7743, or an amino acid sequence having at least 70%, 75%, 80%, 85%, 90%, 95%, or 99% identity thereto. In certain embodiments, the linker comprises a linker sequence of a gene modifying polypeptide as listed in any of Tables T1 or T2, or an amino acid sequence having at least 70%, 75%, 80%, 85%, 90%, 95%, or 99% identity thereto. In certain embodiments, the linker comprises an amino acid sequence as listed in Table 10, or an amino acid sequence having at least 70%, 75%, 80%, 85%, 90%, 95%, or 99% identity thereto.

TABLE 10
Exemplary linker sequences

| Amino Acid Sequence | SEQ ID NO |
|---|---|
| GGS | 5101 |
| GGSGGS | 5102 |
| GGSGGSGGS | 5103 |
| GGSGGSGGSGGS | 5104 |
| GGSGGSGGSGGSGGS | 5105 |
| GGSGGSGGSGGSGGSGGS | 5106 |
| GGGGS | 5107 |
| GGGGSGGGGS | 5108 |
| GGGGSGGGGSGGGGS | 5109 |
| GGGGSGGGGSGGGGSGGGGS | 5110 |
| GGGGSGGGGSGGGGSGGGGSGGGGS | 5111 |
| GGGGSGGGGGGGGSGGGGSGGGGSGGGGS | 5112 |
| GGG | 5113 |
| GGGG | 5114 |
| GGGGG | 5115 |
| GGGGGG | 5116 |
| GGGGGGG | 5117 |
| GGGGGGGG | 5118 |
| GSS | 5119 |
| GSSGSS | 5120 |
| GSSGSSGSS | 5121 |
| GSSGSSGSSGSS | 5122 |
| GSSGSSGSSGSSGSS | 5123 |
| GSSGSSGSSGSSGSSGSS | 5124 |
| EAAAK | 5125 |
| EAAAKEAAAK | 5126 |
| EAAAKEAAAKEAAAK | 5127 |
| EAAAKEAAAKEAAAKEAAAK | 5128 |
| EAAAKEAAAKEAAAKEAAAKEAAAK | 5129 |
| EAAAKEAAAKEAAAKEAAAKEAAAKEAAAK | 5130 |
| PAP | 5131 |
| PAPAP | 5132 |
| PAPAPAP | 5133 |
| PAPAPAPAP | 5134 |
| PAPAPAPAPAP | 5135 |
| PAPAPAPAPAPAP | 5136 |
| GGSGGG | 5137 |
| GGGGGS | 5138 |
| GGSGSS | 5139 |
| GSSGGS | 5140 |
| GGSEAAAK | 5141 |
| EAAAKGGS | 5142 |
| GGSPAP | 5143 |
| PAPGGS | 5144 |
| GGGGSS | 5145 |
| GSSGGG | 5146 |
| GGGEAAAK | 5147 |
| EAAAKGGG | 5148 |
| GGGPAP | 5149 |
| PAPGGG | 5150 |
| GSSEAAAK | 5151 |
| EAAAKGSS | 5152 |
| GSSPAP | 5153 |
| PAPGSS | 5154 |
| EAAAKPAP | 5155 |
| PAPEAAAK | 5156 |
| GGSGGGGSS | 5157 |
| GGSGSSGGG | 5158 |
| GGGGGSGSS | 5159 |
| GGGGSSGGS | 5160 |
| GSSGGSGGG | 5161 |
| GSSGGGGGS | 5162 |
| GGSGGGEAAAK | 5163 |
| GGSEAAAKGGG | 5164 |
| GGGGGSEAAAK | 5165 |
| GGGEAAAKGGS | 5166 |
| EAAAKGGSGGG | 5167 |
| EAAAKGGGGGS | 5168 |
| GGSGGGPAP | 5169 |
| GGSPAPGGG | 5170 |
| GGGGGSPAP | 5171 |
| GGGPAPGGS | 5172 |
| PAPGGSGGG | 5173 |
| PAPGGGGGS | 5174 |
| GGSGSSEAAAK | 5175 |
| GGSEAAAKGSS | 5176 |
| GSSGGSEAAAK | 5177 |
| GSSEAAAKGGS | 5178 |
| EAAAKGGSGSS | 5179 |
| EAAAKGSSGGS | 5180 |
| GGSGSSPAP | 5181 |
| GGSPAPGSS | 5182 |
| GSSGGSPAP | 5183 |
| GSSPAPGGS | 5184 |
| PAPGGSGSS | 5185 |
| PAPGSSGGS | 5186 |
| GGSEAAAKPAP | 5187 |
| GGSPAPEAAAK | 5188 |
| EAAAKGGSPAP | 5189 |
| EAAAKPAPGGS | 5190 |
| PAPGGSEAAAK | 5191 |
| PAPEAAAKGGS | 5192 |
| GGGGSSEAAAK | 5193 |
| GGGEAAAKGSS | 5194 |
| GSSGGGEAAAK | 5195 |
| GSSEAAAKGGG | 5196 |
| EAAAKGGGGSS | 5197 |
| EAAAKGSSGGG | 5198 |
| GGGGSSPAP | 5199 |
| GGGPAPGSS | 5200 |
| GSSGGGPAP | 5201 |
| GSSPAPGGG | 5202 |
| PAPGGGGSS | 5203 |
| PAPGSSGGG | 5204 |
| GGGEAAAKPAP | 5205 |
| GGGPAPEAAAK | 5206 |
| EAAAKGGGPAP | 5207 |
| EAAAKPAPGGG | 5208 |
| PAPGGGEAAAK | 5209 |
| PAPEAAAKGGG | 5210 |
| GSSEAAAKPAP | 5211 |
| GSSPAPEAAAK | 5212 |
| ΕΛΛΛKGSSPAP | 5213 |
| EAAAKPAPGSS | 5214 |
| PAPGSSEAAAK | 5215 |
| PAPEAAAKGSS | 5216 |
| AEAAAKEAAAKEAAAKEAAAKALEAEAAAKEAAAKEA | 5217 |
| AAKEAAAKA | |
| GGGGSEAAAKGGGGS | 5218 |
| EAAAKGGGGSEAAAK | 5219 |
| SGSETPGTSESATPES | 5220 |
| GSAGSAAGSGEF | 5221 |
| SGGSSGGSSGSETPGTSESATPESSGGSSGGSS | 5222 |

In some embodiments, a linker of a gene modifying polypeptide comprises a motif chosen from: (SGGS)ₙ (SEQ ID NO: 5025), (GGGS)ₙ (SEQ ID NO: 5026), (GGGGS)ₙ (SEQ ID NO: 5027), (G)ₙ, (EAAAK)ₙ (SEQ ID NO: 5028), (GGS)ₙ, or (XP)ₙ.

In certain embodiments, the gene modifying polypeptide further comprises a spacer sequence between the linker and the RT domain. In certain embodiments, the spacer sequence between the linker and the RT domain comprises 1, 2, 3, 4, 5, 6, 7, 8, 9, or 10 amino acids. In certain embodiments, the spacer sequence between the linker and the RT domain comprises the amino acid sequence GG.

In certain embodiments, the RT domain comprises a RT domain sequence of a gene modifying polypeptide of any one of SEQ ID NOs: 1-7743, or an amino acid sequence having at least 70%, 75%, 80%, 85%, 90%, 95%, or 99% identity thereto. In certain embodiments, the RT domain comprises a RT domain sequence of a gene modifying polypeptide as listed in any of Tables T1 or T2, or an amino acid sequence having at least 70%, 75%, 80%, 85%, 90%, 95%, or 99% identity thereto. In certain embodiments, the RT domain comprises an amino acid sequence of any one of SEQ ID NOs: 8001-8136, or an amino acid sequence having at least 70%, 75%, 80%, 85%, 90%, 95%, or 99% identity thereto. In some embodiments, the RT domain has a length of about 400-500, 500-600, 600-700, 700-800, 800-900, or 900-1000 amino acids.

In certain embodiments, the gene modifying polypeptide further comprises a spacer sequence between the RT domain and the second NLS. In certain embodiments, the spacer sequence between the RT domain and the second NLS comprises 1, 2, 3, 4, 5, 6, 7, 8, 9, or 10 amino acids. In certain embodiments, the spacer sequence between the RT domain and the second NLS comprises the amino acid sequence AG.

In certain embodiments, the second NLS comprises a second NLS sequence of a gene modifying polypeptide of any one of SEQ ID NOs: 1-7743. In certain embodiments, the second NLS comprises a second NLS sequence of a gene modifying polypeptide as listed in any of Tables T1 or T2. In certain embodiments, the second NLS sequence comprises a plurality of partial NLS sequences. In embodiments, the NLS sequence, e.g., the second NLS sequence, comprises a first partial NLS sequence, e.g., comprising the amino acid sequence KRTADGSEFE (SEQ ID NO: 8198), or an amino acid sequence having at least 70%, 75%, 80%, 85%, 90%, 95%, or 99% identity thereto. In embodiments, the NLS sequence, e.g., the second NLS sequence, comprises a second partial NLS sequence. In embodiments, the NLS sequence, e.g., the second NLS sequence, comprises an SV40A5 NLS, e.g., a bipartite SV40A5 NLS, e.g., comprising the amino acid sequence KRTADGSEFESPKKKAKVE (SEQ ID NO: 8199), or an amino acid sequence having at least 70%, 75%, 80%, 85%, 90%, 95%, or 99% identity thereto. In certain embodiments, the NLS sequence, e.g., the second NLS sequence, comprises the amino acid sequence KRTADGSEFEKRTADGSEFESPKKKAKVE (SEQ ID NO: 8200), or an amino acid sequence having at least 70%, 75%, 80%, 85%, 90%, 95%, or 99% identity thereto.

In certain embodiments, the gene modifying polypeptide further comprises a spacer sequence between the second NLS and the T2A sequence and/or puromycin sequence. In certain embodiments, the spacer sequence between the second NLS and the T2A sequence and/or puromycin sequence comprises 1, 2, 3, 4, 5, 6, 7, 8, 9, or 10 amino acids. In certain embodiments, the spacer sequence between the second NLS and the T2A sequence and/or puromycin sequence comprises the amino acid sequence GSG.

### Linkers and RT Domains

In some embodiments, the gene modifying polypeptide comprises a linker (e.g., as described herein) and an RT domain (e.g., as described herein). In certain embodiments, the gene modifying polypeptide comprises, in N-terminal to C-terminal order, a linker (e.g., as described herein) and an RT domain (e.g., as described herein).

In certain embodiments, the linker comprises a linker sequence as listed in Table 10, or an amino acid sequence having at least 70%, 75%, 80%, 85%, 90%, 95%, or 99% identity thereto. In certain embodiments, the linker comprises a linker sequence of any one of SEQ ID NOs: 1-7743, or an amino acid sequence having at least 70%, 75%, 80%, 85%, 90%, 95%, or 99% identity thereto. In certain embodiments, the linker comprises a linker sequence of any one of SEQ ID NOs: 6001-7743, or an amino acid sequence having at least 70%, 75%, 80%, 85%, 90%, 95%, or 99% identity thereto. In certain embodiments, the linker comprises a linker sequence of any one of SEQ ID NOs: 4501-4541, or an amino acid sequence having at least 70%, 75%, 80%, 85%, 90%, 95%, or 99% identity thereto. In certain embodiments, the linker comprises a linker sequence of an exemplary gene modifying polypeptide listed in any of Tables T1 or T2, or an amino acid sequence having at least 70%, 75%, 80%, 85%, 90%, 95%, or 99% identity thereto. In certain embodiments, the RT domain comprises an RT domain sequence having an amino acid sequence selected from SEQ ID NOs: 8001-8136, or an amino acid sequence having at least 70%, 75%, 80%, 85%, 90%, 95%, or 99% identity thereto. In certain embodiments, the RT domain comprises an RT domain sequence of an exemplary gene modifying polypeptide listed in any of Tables T1 or T2, or an amino acid sequence having at least 70%, 75%, 80%, 85%, 90%, 95%, or 99% identity thereto.

In some embodiments, a gene modifying polypeptide comprises a portion of a gene modifying polypeptide of any one of SEQ ID NOs: 1-7743, wherein the portion comprises a linker and RT domain, or an amino acid sequence having at least 70%, 75%, 80%, 85%, 90%, 95%, or 99% identity to said portion.

In some embodiments, a gene modifying polypeptide comprises a linker of a gene modifying polypeptide of any one of SEQ ID NOs: 1-7743, or an amino acid sequence having at least 70%, 75%, 80%, 85%, 90%, 95%, or 99% identity to said linker. In some embodiments, a gene modifying polypeptide comprises a linker of a gene modifying polypeptide of any one of SEQ ID NOs: 6001-7743, or an amino acid sequence having at least 70%, 75%, 80%, 85%, 90%, 95%, or 99% identity to said linker. In some embodiments, a gene modifying polypeptide comprises a linker of a gene modifying polypeptide of any one of SEQ ID NOs: 4501-4541, or an amino acid sequence having at least 70%, 75%, 80%, 85%, 90%, 95%, or 99% identity to said linker. In some embodiments, a gene modifying polypeptide comprises a linker of a gene modifying polypeptide as listed in any of Tables T1 or T2, or a linker comprising an amino acid sequence having at least 70%, 75%, 80%, 85%, 90%, 95%, or 99% identity thereto.

In some embodiments, a gene modifying polypeptide comprises an RT domain of a gene modifying polypeptide of any one of SEQ ID NOs: 1-7743, or an amino acid sequence having at least 70%, 75%, 80%, 85%, 90%, 95%, or 99% identity to said RT domain. In some embodiments, a gene modifying polypeptide comprises an RT domain of a gene modifying polypeptide of any one of SEQ ID NOs: 6001-7743, or an amino acid sequence having at least 70%, 75%, 80%, 85%, 90%, 95%, or 99% identity said RT domain. In some embodiments, a gene modifying polypeptide comprises an RT domain of a gene modifying polypeptide of any one of SEQ ID NOs: 4501-4541, or an amino acid sequence having at least 70%, 75%, 80%, 85%, 90%, 95%, or 99% identity said RT domain. In some embodiments, a gene modifying polypeptide comprises an RT domain of a gene modifying polypeptide as listed in any of Tables T1 or T2, or an RT domain comprising an amino acid sequence having at least 70%, 75%, 80%, 85%, 90%, 95%, or 99% identity thereto.

In certain embodiments, the linker and the RT domain of a gene modifying polypeptide comprise the amino acid sequences of a linker and RT domain (or amino acid sequences having at least 70%, 75%, 80%, 85%, 90%, 95%, or 99% identity thereto) of a gene modifying polypeptide having the amino acid sequence of any one of SEQ ID NOs: 1-7743. In certain embodiments, the linker and the RT domain of a gene modifying polypeptide comprise amino acid sequences of a linker and RT domain having at least 80% identity to the linker and RT domains of any one of SEQ ID NOs: 1-7743. In certain embodiments, the linker and the RT domain of a gene modifying polypeptide comprise amino acid sequences of a linker and RT domain having at least 90% identity to the linker and RT domains of any one of SEQ ID NOs: 1-7743. In certain embodiments, the linker and the RT domain of a gene modifying polypeptide comprise amino acid sequences of a linker and RT domain having at least 95% identity to the linker and RT domains of any one of SEQ ID NOs: 1-7743. In certain embodiments, the linker and the RT domain of a gene modifying polypeptide comprise amino acid sequences of a linker and RT domain having at least 99% identity to the linker and RT domains of any one of SEQ ID NOs: 1-7743. In certain embodiments, the linker and the RT domain of a gene modifying polypeptide comprise the amino acid sequences of a linker and RT domain (or amino acid sequences having at least 70%, 75%, 80%, 85%, 90%, 95%, or 99% identity thereto) of a gene modifying polypeptide having the amino acid sequence of any one of SEQ ID NOs: 6001-7743. In certain embodiments, the linker and the RT domain of a gene modifying polypeptide comprise the amino acid sequences of a linker and RT domain (or amino acid sequences having at least 70%, 75%, 80%, 85%, 90%, 95%, or 99% identity thereto) of a gene modifying polypeptide having the amino acid sequence of any one of SEQ ID NOs: 4501-4541. In certain embodiments, the linker and the RT domain of a gene modifying polypeptide comprise the amino acid sequences of a linker and RT domain (or amino acid sequences having at least 70%, 75%, 80%, 85%, 90%, 95%, or 99% identity thereto) from a single row of any of Tables T1 or T2 (e.g., from a single exemplary gene modifying polypeptide as listed in any of Tables T1 or T2).

In certain embodiments, the linker and the RT domain of a gene modifying polypeptide comprise the amino acid sequences of a linker and RT domain (or amino acid sequences having at least 70%, 75%, 80%, 85%, 90%, 95%, or 99% identity thereto) from two different amino acid sequences selected from SEQ ID NOs: 1-7743. In certain embodiments, the linker and the RT domain of a gene modifying polypeptide comprise the amino acid sequences of a linker and RT domain (or amino acid sequences having at least 70%, 75%, 80%, 85%, 90%, 95%, or 99% identity thereto) from different rows of any of Tables T1 or T2.

In certain embodiments, the gene modifying polypeptide further comprises a first NLS (e.g., a 5′ NLS), e.g., as described herein. In certain embodiments, the gene modifying polypeptide further comprises a second NLS (e.g., a 3′ NLS), e.g., as described herein. In certain embodiments, the gene modifying polypeptide further comprises an N-terminal methionine residue.

### RT Families and Mutants

In certain embodiments, a gene modifying polypeptide comprises the amino acid sequence of an RT domain sequence from a family selected from: AVIRE, BAEVM, FFV, FLV, FOAMV, GALV, KORV, MLVAV, MLVBM, MLVCB, MLVFF, MLVMS, PERV, SFV1, SFV3L, WMSV, XMRV6, BLVAU, BLVJ, HTLIA, HTLIC, HTL1L, HTL32, HTL3P, HTLV2, JSRV, MLVF5, MLVRD, MMTVB, MPMV, SFVCP, SMRVH, SRV1, SRV2, and WDSV. In certain embodiments, a gene modifying polypeptide comprises the amino acid sequence of an RT domain sequence from a family selected from: AVIRE, BAEVM, FFV, FLV, FOAMV, GALV, KORV, MLVAV, MLVBM, MLVCB, MLVFF, MLVMS, PERV, SFV1, SFV3L, WMSV, and XMRV6.

In certain embodiments, a gene modifying polypeptide comprises the amino acid sequence of an RT domain sequence from an MLVMS RT domain. In embodiments, the amino acid sequence of an RT domain sequence comprises one or more point mutations as listed in column 1 of Table M1, or a point mutation corresponding thereto. In embodiments, the amino acid sequence of an RT domain sequence comprises one or more point mutations as listed in column 3 of Table M1 (Gen1 MLVMS), or a point mutation corresponding thereto. In embodiments, the amino acid sequence of an RT domain sequence comprises one or more point mutations at an amino acid position of the RT domain as listed in columns 1 and 2 of Table M2, or an amino acid position corresponding thereto.

In certain embodiments, a gene modifying polypeptide comprises the amino acid sequence of an RT domain sequence from an AVIRE RT domain. In embodiments, the amino acid sequence of an RT domain sequence comprises one or more point mutations as listed in column 2 of Table M1, or a point mutation corresponding thereto. In embodiments, the amino acid sequence of an RT domain sequence comprises one or more point mutations as listed in column 4 of Table M1 (Gen2 AVIRE), or a point mutation corresponding thereto. In embodiments, the amino acid sequence of an RT domain sequence comprises one or more point mutations at an amino acid position of the RT domain as listed in columns 3 and 4 of Table M2, or an amino acid position corresponding thereto. In certain embodiments, the RT domain comprises an IENSSP (e.g., at the C-terminus).

TABLE M1
Exemplary point mutations in MLVMS and AVIRE RT domains

| RT-linker | | Gen1 | |
|---|---|---|---|
| filing | Corresponding | MLVMS | Gen2 AVIRE |
| (MLVMS) | AVIRE | (PLV4921) | (PLV10990) |
| | | H8Y | |
| P51L | Q51L | | |
| S67R | T67R | | |
| E67K | E67K | | |
| E69K | E69K | | |
| T197A | T197A | | |
| D200N | D200N | D200N | D200N |
| H204R | N204R | | |
| E302K | E302K | | |
| | | T306K | T306K |
| F309N | Y309N | | |
| W313F | W313F | W313F | W313F |
| T330P | G330P | T330P | G330P |
| L435G | T436G | | |
| N454K | N455K | | |
| D524G | D526G | | |
| E562Q | E564Q | | |
| D583N | D585N | | |
| H594Q | H596Q | | |
| L603W | L605W | L603W | L605W |
| D653N | D655N | | |
| L671P | L673P | | |

IENSSP at C-term

TABLE M2
Positions that can be mutated in exemplary MLVMS and AVIRE
RT domains
WT residue & position

| MLVMS | MLVMS | | AVIRE |
|---|---|---|---|
| aa | position # * | AVIRE aa | position # * |
| H | 8 | Y | 8 |
| P | 51 | Q | 51 |
| S | 67 | T | 67 |
| E | 69 | E | 69 |
| T | 197 | T | 197 |
| D | 200 | D | 200 |
| H | 204 | N | 204 |
| E | 302 | E | 302 |
| T | 306 | T | 306 |
| F | 309 | Y | 309 |
| W | 313 | W | 313 |
| T | 330 | G | 330 |
| L | 435 | T | 436 |
| N | 454 | N | 455 |
| D | 524 | D | 526 |
| E | 562 | E | 564 |
| D | 583 | D | 585 |
| H | 594 | H | 596 |
| L | 603 | L | 605 |
| D | 653 | D | 655 |
| L | 671 | S | 673 |

In certain embodiments, a gene modifying polypeptide comprises a gamma retrovirus derived RT domain. In certain embodiments, the gamma retrovirus-derived RT domain of a gene modifying polypeptide comprises the amino acid sequence of an RT domain sequence from a family selected from: AVIRE, BAEVM, FFV, FLV, FOAMV, GALV, KORV, MLVAV, MLVBM, MLVCB, MLVFF, MLVMS, PERV, SFV1, SFV3L, WMSV, and XMRV6. In some embodiments, the gamma retrovirus-derived RT domain of a gene modifying polypeptide is not derived from PERV. In some embodiments, said RT includes one, two, three, four, five, six or more mutations corresponding to mutations D200N, L603W, T330P, D524G, E562Q, D583N, P51L, S67R, E67K, T197A, H204R, E302K, F309N, W313F, L435G, N454K, H594Q, L671P, E69K, H8Y, T306K, or D653N in the RT domain of murine leukemia virus reverse transcriptase. In some embodiments, the gene modifying polypeptide further comprises a linker having at least 99% identity to a linker domains of any one of SEQ ID NOs: 1-7743. In some embodiments, the gene modifying polypeptide further comprises a linker having at least 99% or 100% identity to SEQ ID NO: 5217.

In embodiments, the RT domain comprises the amino acid sequence of an RT domain of an AVIRE RT (e.g., an AVIRE_P03360 sequence, e.g., SEQ ID NO: 8001), or an amino acid sequence having at least 70%, 75%, 80%, 85%, 90%, 95%, or 99% identity thereto. In some embodiments, the RT domain comprises the amino acid sequence of an AVIRE RT further comprising one, two, three, four, or five mutations selected from the group consisting of D200N, G330P, L605W, T306K, and W313F, or a corresponding position in a homologous RT domain. In some embodiments, the RT domain comprises the amino acid sequence of an AVIRE RT further comprising one, two, or three mutations selected from the group consisting of D200N, G330P, and L605W, or a corresponding position in a homologous RT domain.

In embodiments, the RT domain comprises the amino acid sequence of an RT domain of a BAEVM RT (e.g., an BAEVM_P10272 sequence, e.g., SEQ ID NO: 8004), or an amino acid sequence having at least 70%, 75%, 80%, 85%, 90%, 95%, or 99% identity thereto. In some embodiments, the RT domain comprises the amino acid sequence of a BAEVM RT further comprising one, two, three, four, or five mutations selected from the group consisting of D198N, E328P, L602W, T304K, and W311F, or a corresponding position in a homologous RT domain. In some embodiments, the RT domain comprises the amino acid sequence of a BAEVM RT further comprising one, two, or three mutations selected from the group consisting of D198N, E328P, and L602W, or a corresponding position in a homologous RT domain.

In embodiments, the RT domain comprises the amino acid sequence of an RT domain of an FFV RT (e.g., an FFV_093209 sequence, e.g., SEQ ID NO: 8012), or an amino acid sequence having at least 70%, 75%, 80%, 85%, 90%, 95%, or 99% identity thereto. In some embodiments, the RT domain comprises the amino acid sequence of an FFV RT further comprising one, two, three, or four mutations selected from the group consisting of D21N, T293N, T419P, and L393K, or a corresponding position in a homologous RT domain. In some embodiments, the RT domain comprises the amino acid sequence of an FFV RT further comprising one, two, or three mutations selected from the group consisting of D21N, T293N, and T419P, or a corresponding position in a homologous RT domain. In some embodiments, the RT domain comprises the amino acid sequence of an FFV RT further comprising the mutation D21N. In some embodiments, the RT domain comprises the amino acid sequence of an FFV RT further comprising one, two, or three mutations selected from the group consisting of T207N, T333P, and L307K, or a corresponding position in a homologous RT domain. In some embodiments, the RT domain comprises the amino acid sequence of an FFV RT further comprising one or two mutations selected from the group consisting of T207N and T333P, or a corresponding position in a homologous RT domain.

In embodiments, the RT domain comprises the amino acid sequence of an RT domain of an FLV RT (e.g., an FLV_P10273 sequence, e.g., SEQ ID NO: 8019), or an amino acid sequence having at least 70%, 75%, 80%, 85%, 90%, 95%, or 99% identity thereto. In some embodiments, the RT domain comprises the amino acid sequence of an FLV RT further comprising one, two, three, or four mutations selected from the group consisting of D199N, L602W, T305K, and W312F, or a corresponding position in a homologous RT domain. In some embodiments, the RT domain comprises the amino acid sequence of an FLV RT further comprising one or two mutations selected from the group consisting of D199N and L602W, or a corresponding position in a homologous RT domain.

In embodiments, the RT domain comprises the amino acid sequence of an RT domain of a FOAMV RT (e.g., an FOAMV_P14350 sequence, e.g., SEQ ID NO: 8021), or an amino acid sequence having at least 70%, 75%, 80%, 85%, 90%, 95%, or 99% identity thereto. In some embodiments, the RT domain comprises the amino acid sequence of an FOAMV RT further comprising one, two, three, or four mutations selected from the group consisting of D24N, T296N, S420P, and L396K, or a corresponding position in a homologous RT domain. In some embodiments, the RT domain comprises the amino acid sequence of an FOAMV RT further comprising one, two, or three mutations selected from the group consisting of D24N, T296N, and S420P, or a corresponding position in a homologous RT domain. In some embodiments, the RT domain comprises the amino acid sequence of an FOAMV RT further comprising the mutation D24N, or a corresponding position in a homologous RT domain. In some embodiments, the RT domain comprises the amino acid sequence of an FOAMV RT further comprising one, two, or three mutations selected from the group consisting of T207N, S331P, and L307K, or a corresponding position in a homologous RT domain. In some embodiments, the RT domain comprises the amino acid sequence of an FOAMV RT further comprising one or two mutations selected from the group consisting of T207N and S331P, or a corresponding position in a homologous RT domain.

In embodiments, the RT domain comprises the amino acid sequence of an RT domain of a GALV RT (e.g., an GALV_P21414 sequence, e.g., SEQ ID NO: 8027), or an amino acid sequence having at least 70%, 75%, 80%, 85%, 90%, 95%, or 99% identity thereto. In some embodiments, the RT domain comprises the amino acid sequence of a GALV RT further comprising one, two, three, four, or five mutations selected from the group consisting of D198N, E328P, L600W, T304K, and W311F, or a corresponding position in a homologous RT domain. In some embodiments, the RT domain comprises the amino acid sequence of a GALV RT further comprising one, two, or three mutations selected from the group consisting of D198N, E328P, and L600W, or a corresponding position in a homologous RT domain.

In embodiments, the RT domain comprises the amino acid sequence of an RT domain of a KORV RT (e.g., an KORV_Q9TTC1 sequence, e.g., SEQ ID NO: 8047), or an amino acid sequence having at least 70%, 75%, 80%, 85%, 90%, 95%, or 99% identity thereto. In some embodiments, the RT domain comprises the amino acid sequence of a GALV RT further comprising one, two, three, four, five, or six mutations selected from the group consisting of D32N, D322N, E452P, L274W, T428K, and W435F, or a corresponding position in a homologous RT domain. In some embodiments, the RT domain comprises the amino acid sequence of a GALV RT further comprising one, two, three, or four mutations selected from the group consisting of D32N, D322N, E452P, and L274W, or a corresponding position in a homologous RT domain. In some embodiments, the RT domain comprises the amino acid sequence of a GALV RT further comprising the mutation D32N. In some embodiments, the RT domain comprises the amino acid sequence of a KORV RT further comprising one, two, three, four, or five mutations selected from the group consisting of D231N, E361P, L633W, T337K, and W344F, or a corresponding position in a homologous RT domain. In some embodiments, the RT domain comprises the amino acid sequence of a KORV RT further comprising one, two, or three mutations selected from the group consisting of D23IN, E361P, and L633W, or a corresponding position in a homologous RT domain.

In embodiments, the RT domain comprises the amino acid sequence of an RT domain of a MLVAV RT (e.g., an MLVAV_P03356 sequence, e.g., SEQ ID NO: 8053), or an amino acid sequence having at least 70%, 75%, 80%, 85%, 90%, 95%, or 99% identity thereto. In some embodiments, the RT domain comprises the amino acid sequence of a MLVAV RT further comprising one, two, three, four, or five mutations selected from the group consisting of D200N, T330P, L603W, T306K, and W313F, or a corresponding position in a homologous RT domain. In some embodiments, the RT domain comprises the amino acid sequence of a MLVAV RT further comprising one, two, or three mutations selected from the group consisting of D200N, T330P, and L603W, or a corresponding position in a homologous RT domain.

In embodiments, the RT domain comprises the amino acid sequence of an RT domain of a MLVBM RT (e.g., an MLVBM_Q7SVK7 sequence, e.g., SEQ ID NO: 8056), or an amino acid sequence having at least 70%, 75%, 80%, 85%, 90%, 95%, or 99% identity thereto. In some embodiments, the RT domain comprises the amino acid sequence of a MLVBM RT further comprising one, two, three, four, or five mutations selected from the group consisting of D199N, T329P, L602W, T305K, and W312F, or a corresponding position in a homologous RT domain. In some embodiments, the RT domain comprises the amino acid sequence of a MLVBM RT further comprising one, two, and three mutations selected from the group consisting of D200N, T330P, and L603W, or a corresponding position in a homologous RT domain.

In embodiments, the RT domain comprises the amino acid sequence of an RT domain of a MLVCB RT (e.g., an MLVCB_P08361 sequence, e.g., SEQ ID NO: 8062), or an amino acid sequence having at least 70%, 75%, 80%, 85%, 90%, 95%, or 99% identity thereto. In some embodiments, the RT domain comprises the amino acid sequence of a MLVCB RT further comprising one, two, three, four, or five mutations selected from the group consisting of D200N, T330P, L603W, T306K, and W313F, or a corresponding position in a homologous RT domain. In some embodiments, the RT domain comprises the amino acid sequence of a MLVCB RT further comprising one, two, and three mutations selected from the group consisting of D200N, T330P, and L603W, or a corresponding position in a homologous RT domain.

In embodiments, the RT domain comprises the amino acid sequence of an RT domain of a MLVFF RT, or an amino acid sequence having at least 70%, 75%, 80%, 85%, 90%, 95%, or 99% identity thereto. In some embodiments, the RT domain comprises the amino acid sequence of a MLVFF RT further comprising one, two, three, four, or five mutations selected from the group consisting of D200N, T330P, L603W, T306K, and W313F, or a corresponding position in a homologous RT domain. In some embodiments, the RT domain comprises the amino acid sequence of a MLVFF RT further comprising one, two, and three mutations selected from the group consisting of D200N, T330P, and L603W, or a corresponding position in a homologous RT domain.

In embodiments, the RT domain comprises the amino acid sequence of an RT domain of a MLVMS RT (e.g., an MLVMS_reference sequence, e.g., SEQ ID NO: 8370; or an MLVMS_P03355 sequence, e.g., SEQ ID NO: 8070), or an amino acid sequence having at least 70%, 75%, 80%, 85%, 90%, 95%, or 99% identity thereto. In some embodiments, the RT domain comprises the amino acid sequence of a MLVMS RT further comprising one, two, three, four, five, or six mutations selected from the group consisting of D200N, T330P, L603W, T306K, W313F, and H8Y, or a corresponding position in a homologous RT domain. In some embodiments, the RT domain comprises the amino acid sequence of a MLVMS RT further comprising one, two, three, four, or five mutations selected from the group consisting of D200N, T330P, L603W, T306K, and W313F, or a corresponding position in a homologous RT domain. In some embodiments, the RT domain comprises the amino acid sequence of a MLVMS RT further comprising one, two, or three mutations selected from the group consisting of D200N, T330P, and L603W, or a corresponding position in a homologous RT domain.

In embodiments, the RT domain comprises the amino acid sequence of an RT domain of a PERV RT (e.g., an PERV_Q4VFZ2 sequence, e.g., SEQ ID NO: 8099), or an amino acid sequence having at least 70%, 75%, 80%, 85%, 90%, 95%, or 99% identity thereto. In some embodiments, the RT domain comprises the amino acid sequence of a PERV RT further comprising one, two, three, four, or five mutations selected from the group consisting of D196N, E326P, L599W, T302K, and W309F, or a corresponding position in a homologous RT domain. In some embodiments, the RT domain comprises the amino acid sequence of a PERV RT further comprising one, two, or three mutations selected from the group consisting of D196N, E326P, and L599W, or a corresponding position in a homologous RT domain.

In embodiments, the RT domain comprises the amino acid sequence of an RT domain of a SFV1 RT (e.g., an SFV1_P23074 sequence, e.g., SEQ ID NO: 8105), or an amino acid sequence having at least 70%, 75%, 80%, 85%, 90%, 95%, or 99% identity thereto. In some embodiments, the RT domain comprises the amino acid sequence of a SFV1 RT further comprising one, two, three, or four mutations selected from the group consisting of D24N, T296N, N420P, and L396K, or a corresponding position in a homologous RT domain. In some embodiments, the RT domain comprises the amino acid sequence of a SFV1 RT further comprising one, two, or three mutations selected from the group consisting of D24N, T296N, and N420P, or a corresponding position in a homologous RT domain. In some embodiments, the RT domain comprises the amino acid sequence of a SFV1 RT further comprising the D24N, or a corresponding position in a homologous RT domain.

In embodiments, the RT domain comprises the amino acid sequence of an RT domain of a SFV3L RT (e.g., an SFV3L_P27401 sequence, e.g., SEQ ID NO: 8111), or an amino acid sequence having at least 70%, 75%, 80%, 85%, 90%, 95%, or 99% identity thereto. In some embodiments, the RT domain comprises the amino acid sequence of a SFV3L RT further comprising one, two, three, or four mutations selected from the group consisting of D24N, T296N, N422P, and L396K, or a corresponding position in a homologous RT domain. In some embodiments, the RT domain comprises the amino acid sequence of a SFV3L RT further comprising one, two, or three mutations selected from the group consisting of D24N, T296N, and N422P, or a corresponding position in a homologous RT domain. In some embodiments, the RT domain comprises the amino acid sequence of a SFV3L RT further comprising the mutation D24N, or a corresponding position in a homologous RT domain. In some embodiments, the RT domain comprises the amino acid sequence of a SFV3L RT further comprising one, two, or three mutations selected from the group consisting of T307N, N333P, and L307K, or a corresponding position in a homologous RT domain. In some embodiments, the RT domain comprises the amino acid sequence of a SFV3L RT further comprising one or two mutations selected from the group consisting of T307N and N333P, or a corresponding position in a homologous RT domain.

In embodiments, the RT domain comprises the amino acid sequence of an RT domain of a WMSV RT (e.g., an WMSV_P03359 sequence, e.g., SEQ ID NO: 8131), or an amino acid sequence having at least 70%, 75%, 80%, 85%, 90%, 95%, or 99% identity thereto. In some embodiments, the RT domain comprises the amino acid sequence of a WMSV RT further comprising one, two, three, four, or five mutations selected from the group consisting of D198N, E328P, L600W, T304K, and W311F, or a corresponding position in a homologous RT domain. In some embodiments, the RT domain comprises the amino acid sequence of a WMSV RT further comprising one, two, or three mutations selected from the group consisting of D198N, E328P, and L600W, or a corresponding position in a homologous RT domain.

In embodiments, the RT domain comprises the amino acid sequence of an RT domain of a XMRV6 RT (e.g., an XMRV6_AIZ651 sequence, e.g., SEQ ID NO: 8134), or an amino acid sequence having at least 70%, 75%, 80%, 85%, 90%, 95%, or 99% identity thereto. In some embodiments, the RT domain comprises the amino acid sequence of a XMRV6 RT further comprising one, two, three, four, or five mutations selected from the group consisting of D200N, T330P, L603W, T306K, and W313F, or a corresponding position in a homologous RT domain. In some embodiments, the RT domain comprises the amino acid sequence of a XMRV6 RT further comprising one, two, or three mutations selected from the group consisting of D200N, T330P, and L603W, or a corresponding position in a homologous RT domain.

In certain embodiments, the RT domain of a gene modifying polypeptide comprises the amino acid sequence of an RT domain of an AVIRE RT, or an amino acid sequence having at least 70%, 75%, 80%, 85%, 90%, 95%, or 99% identity thereto. In embodiments, the RT domain comprises the amino acid sequence of an RT domain comprised in a sequence listed in column 1 of Table A5, or an amino acid sequence having at least 70%, 75%, 80%, 85%, 90%, 95%, or 99% identity thereto. In some embodiments, the gene modifying polypeptide further comprises a linker having at least 99% or 100% identity to SEQ ID NO: 5217.

In certain embodiments, the RT domain of a gene modifying polypeptide comprises the amino acid sequence of an RT domain of an MLVMS RT, or an amino acid sequence having at least 70%, 75%, 80%, 85%, 90%, 95%, or 99% identity thereto. In embodiments, the RT domain comprises the amino acid sequence of an RT domain comprised in a sequence listed in any of columns 2-6 of Table A5, or an amino acid sequence having at least 70%, 75%, 80%, 85%, 90%, 95%, or 99% identity thereto. In some embodiments, the gene modifying polypeptide further comprises a linker having at least 99% or 100% identity to SEQ ID NO: 5217.

TABLE A5
Exemplary gene modifying polypeptides comprising
an AVIRE RT domain or an MLVMS RT domain.

| | |
|---|---|
| AVIRE SEQ ID | |
| NOs: | MLVMS SEQ ID NOS: |

| | | | | | |
|---|---|---|---|---|---|
| 1 | 2704 | 3007 | 3038 | 2638 | 2930 |
| 2 | 2706 | 3007 | 3038 | 2639 | 2930 |
| 3 | 2708 | 3008 | 3039 | 2639 | 2931 |
| 4 | 2709 | 3008 | 3039 | 2640 | 2931 |
| 5 | 2709 | 3009 | 3040 | 2640 | 2932 |
| 6 | 2710 | 3010 | 3040 | 2641 | 2932 |
| 7 | 2957 | 3010 | 3041 | 2641 | 2933 |
| 9 | 2957 | 3011 | 3041 | 2642 | 2933 |
| 10 | 2958 | 3012 | 3042 | 2642 | 2934 |
| 12 | 2959 | 3012 | 3042 | 2643 | 2934 |
| 13 | 2960 | 3013 | 3043 | 2643 | 2935 |
| 14 | 2962 | 3013 | 3043 | 2644 | 2935 |
| 6076 | 6042 | 3014 | 3044 | 2644 | 2936 |
| 6143 | 6068 | 3014 | 3044 | 2645 | 2936 |
| 6200 | 6097 | 3015 | 3045 | 2645 | 2937 |
| 6254 | 6136 | 3015 | 3045 | 2646 | 2937 |
| 6274 | 6156 | 3016 | 3046 | 2646 | 2938 |
| 6315 | 6215 | 3016 | 3046 | 2647 | 2938 |
| 6328 | 6216 | 3017 | 3047 | 2647 | 2939 |
| 6337 | 6301 | 3018 | 3047 | 2648 | 2939 |
| 6403 | 6352 | 3018 | 3048 | 2648 | 2940 |
| 6420 | 6365 | 3019 | 3048 | 2649 | 2940 |
| 6440 | 6411 | 3019 | 3049 | 2649 | 2941 |
| 6513 | 6436 | 3020 | 3049 | 2650 | 2941 |
| 6552 | 6458 | 3020 | 3050 | 2650 | 2942 |
| 6613 | 6459 | 3021 | 3051 | 2651 | 2942 |
| 6671 | 6524 | 3021 | 3051 | 2651 | 2943 |
| 6822 | 6562 | 3022 | 3052 | 2652 | 2943 |
| 6840 | 6563 | 3023 | 3052 | 2652 | 2944 |
| 6884 | 6699 | 3023 | 3053 | 2653 | 2945 |
| 6907 | 6865 | 3024 | 3053 | 2653 | 2945 |
| 6970 | 7022 | 3024 | 3054 | 2654 | 2946 |
| 7025 | 7037 | 3025 | 3054 | 2655 | 2946 |
| 7052 | 7088 | 3025 | 3055 | 2655 | 2947 |
| 7078 | 7116 | 3026 | 3055 | 2656 | 2947 |
| 7243 | 7175 | 3026 | 3056 | 2656 | 2948 |
| 7253 | 7200 | 3027 | 3056 | 2657 | 2948 |
| 7318 | 7206 | 3027 | 3057 | 2657 | 2949 |
| 7379 | 7277 | 3028 | 3057 | 2658 | 2949 |
| 7486 | 7294 | 3028 | 3058 | 2658 | 2950 |
| 7524 | 7330 | 3029 | 3058 | 2659 | 2950 |
| 7668 | 7411 | 3030 | 3059 | 2659 | 2951 |
| 7680 | 7455 | 3030 | 3059 | 2660 | 2951 |
| 7720 | 7477 | 3031 | 3060 | 2660 | 2952 |
| 1137 | 7511 | 3031 | 3060 | 2661 | 2952 |
| 1138 | 7538 | 3032 | 3061 | 2661 | 2953 |
| 1139 | 7559 | 3032 | 3061 | 2662 | 2953 |
| 1140 | 7560 | 3033 | 3062 | 2662 | 2954 |
| 1141 | 7593 | 3033 | 3062 | 2663 | 2954 |
| 1142 | 7594 | 3034 | 3063 | 2663 | 2955 |
| 1143 | 7607 | 3034 | 3063 | 2664 | 2955 |
| 1144 | 7623 | 6025 | 3064 | 2664 | 6485 |
| 1145 | 7638 | 6041 | 3064 | 2665 | 6486 |
| 1146 | 7717 | 6043 | 3065 | 2665 | 6504 |
| 1147 | 7731 | 6098 | 3065 | 2666 | 6505 |
| 1148 | 7732 | 6099 | 3066 | 2666 | 6595 |
| 1149 | 2711 | 6180 | 3066 | 2667 | 6596 |
| 1150 | 2711 | 6182 | 3067 | 2667 | 6751 |
| 1151 | 2712 | 6237 | 3067 | 2668 | 6752 |
| 1152 | 2712 | 6238 | 3068 | 2668 | 6777 |
| 1153 | 2713 | 6311 | 3068 | 2669 | 6778 |
| 1154 | 2713 | 6312 | 3069 | 2669 | 7172 |
| 1155 | 2714 | 6578 | 3069 | 2670 | 7174 |
| 1156 | 2714 | 6579 | 3070 | 2670 | 7313 |
| 1157 | 2715 | 6663 | 3070 | 2671 | 7314 |
| 1158 | 2715 | 6664 | 3071 | 2671 | |
| 1159 | 2716 | 6708 | 3071 | 2672 | |
| 1160 | 2716 | 6709 | 3072 | 2672 | |
| 1161 | 2717 | 6809 | 3072 | 2673 | |
| 1162 | 2717 | 6831 | 3073 | 2673 | |
| 1163 | 2718 | 6832 | 3073 | 2674 | |
| 1164 | 2718 | 6864 | 3074 | 2674 | |
| 1165 | 2719 | 6866 | 3074 | 2675 | |
| 1166 | 2719 | 7089 | 3075 | 2675 | |
| 1167 | 2720 | 7157 | 3075 | 2676 | |
| 6015 | 2720 | 7159 | 3076 | 2676 | |
| 6029 | 2721 | 7173 | 3076 | 2677 | |
| 6045 | 2721 | 7176 | 3077 | 2677 | |
| 6077 | 2722 | 7293 | 3077 | 2678 | |
| 6129 | 2722 | 7295 | 3078 | 2678 | |
| 6144 | 2723 | 7343 | 3078 | 2679 | |
| 6164 | 2723 | 7393 | 3079 | 2680 | |
| 6201 | 2724 | 7394 | 3079 | 2680 | |
| 6227 | 2724 | 7425 | 3080 | 2681 | |
| 6244 | 2725 | 7426 | 3080 | 2681 | |
| 6250 | 2725 | 7444 | 3081 | 2682 | |
| 6264 | 2726 | 7445 | 3081 | 2682 | |
| 6289 | 2726 | 7476 | 3082 | 2683 | |
| 6304 | 2727 | 7478 | 3082 | 2683 | |
| 6316 | 2727 | 7496 | 3083 | 2684 | |
| 6384 | 2728 | 7497 | 3083 | 2684 | |
| 6421 | 2728 | 7537 | 3084 | 2685 | |
| 6441 | 2729 | 7539 | 3084 | 2685 | |
| 6492 | 2729 | 2780 | 3085 | 2686 | |
| 6514 | 2730 | 2780 | 3085 | 2686 | |
| 6530 | 2730 | 2781 | 3086 | 2687 | |
| 6569 | 2731 | 2781 | 3086 | 2687 | |
| 6584 | 2731 | 2782 | 3087 | 2688 | |
| 6621 | 2732 | 2782 | 3087 | 2688 | |
| 6651 | 2732 | 2783 | 3088 | 2689 | |
| 6659 | 2733 | 2783 | 3088 | 2689 | |
| 6683 | 2734 | 2784 | 3089 | 2690 | |
| 6703 | 2734 | 2784 | 3089 | 2690 | |
| 6727 | 2735 | 2785 | 3090 | 2691 | |
| 6732 | 2735 | 2785 | 3090 | 2692 | |
| 6745 | 2736 | 2786 | 3091 | 2692 | |
| 6755 | 2736 | 2786 | 3091 | 2693 | |
| 6784 | 2737 | 2787 | 3092 | 2693 | |
| 6817 | 2737 | 2787 | 3092 | 2694 | |
| 6823 | 2738 | 2788 | 3093 | 2694 | |
| 6841 | 2739 | 2788 | 3093 | 2695 | |
| 6871 | 2740 | 2789 | 3094 | 2695 | |
| 6885 | 2740 | 2789 | 3095 | 2696 | |
| 6898 | 2741 | 2790 | 3095 | 2696 | |
| 6908 | 2741 | 2790 | 3096 | 2697 | |
| 6933 | 2742 | 2791 | 3096 | 2697 | |
| 6971 | 2742 | 2791 | 3097 | 2698 | |
| 7009 | 2743 | 2792 | 3097 | 2698 | |
| 7018 | 2743 | 2792 | 3098 | 2699 | |
| 7045 | 2744 | 2793 | 3098 | 2699 | |
| 7053 | 2744 | 2793 | 3099 | 2700 | |
| 7068 | 2745 | 2794 | 3099 | 2700 | |
| 7079 | 2745 | 2794 | 3100 | 2701 | |
| 7096 | 2746 | 2795 | 3100 | 2701 | |
| 7104 | 2746 | 2795 | 3101 | 2702 | |
| 7122 | 2747 | 2796 | 3101 | 2702 | |
| 7151 | 2747 | 2796 | 3102 | 2703 | |
| 7163 | 2748 | 2797 | 3102 | 2703 | |
| 7181 | 2748 | 2797 | 3103 | 2862 | |
| 7244 | 2749 | 2798 | 3103 | 2862 | |
| 7273 | 2750 | 2798 | 3104 | 2863 | |
| 7319 | 2750 | 2799 | 3104 | 2863 | |
| 7336 | 2751 | 2799 | 3105 | 2864 | |
| 7380 | 2751 | 2800 | 3105 | 2864 | |
| 7402 | 2752 | 2800 | 3106 | 2865 | |
| 7462 | 2752 | 2801 | 3106 | 2865 | |
| 7487 | 2753 | 2801 | 3107 | 2866 | |
| 7525 | 2753 | 2802 | 3107 | 2866 | |
| 7569 | 2754 | 2802 | 3108 | 2867 | |
| 7626 | 2754 | 2803 | 3108 | 2867 | |
| 7689 | 2755 | 2803 | 3109 | 2868 | |
| 7707 | 2755 | 2804 | 3109 | 2868 | |
| 7721 | 2756 | 2804 | 3110 | 2869 | |
| 1371 | 2756 | 2805 | 3110 | 2869 | |
| 1372 | 2757 | 2805 | 3111 | 2870 | |
| 1373 | 2758 | 2806 | 3111 | 2870 | |
| 1374 | 2758 | 2806 | 3112 | 2871 | |
| 1375 | 2759 | 2807 | 3112 | 2871 | |
| 1376 | 2759 | 2807 | 3113 | 2872 | |
| 1377 | 2760 | 2808 | 3113 | 2872 | |
| 1378 | 2760 | 2808 | 3114 | 2873 | |
| 1379 | 2761 | 2809 | 3114 | 2873 | |
| 1380 | 2761 | 2809 | 3115 | 2874 | |
| 1381 | 2762 | 2810 | 3115 | 2874 | |
| 1382 | 2762 | 2810 | 3116 | 2875 | |
| 1383 | 2763 | 2811 | 3116 | 2875 | |
| 1384 | 2763 | 2811 | 3117 | 2876 | |
| 1385 | 2764 | 2812 | 3117 | 2876 | |
| 1386 | 2764 | 2812 | 3118 | 2877 | |
| 1387 | 2765 | 2813 | 3118 | 2877 | |
| 1388 | 2765 | 2813 | 3119 | 2878 | |
| 1389 | 2766 | 2814 | 3119 | 2878 | |
| 1390 | 2766 | 2814 | 3120 | 2879 | |
| 1391 | 2767 | 2815 | 3120 | 2879 | |
| 1392 | 2767 | 2815 | 3121 | 2880 | |
| 1393 | 2768 | 2816 | 3121 | 2880 | |
| 1394 | 2768 | 2816 | 3122 | 2881 | |
| 1395 | 2769 | 2817 | 3122 | 2881 | |
| 1396 | 2769 | 2817 | 3123 | 2882 | |
| 1397 | 2770 | 2818 | 3123 | 2882 | |
| 1398 | 2770 | 2818 | 3124 | 2883 | |
| 1399 | 2771 | 2819 | 3124 | 2883 | |
| 1400 | 2771 | 2819 | 3125 | 2884 | |
| 1401 | 2772 | 2820 | 3125 | 2884 | |
| 1402 | 2773 | 2820 | 3126 | 2885 | |
| 1403 | 2773 | 2821 | 3126 | 2885 | |
| 1404 | 2774 | 2821 | 3127 | 2886 | |
| 1405 | 2774 | 2822 | 3127 | 2886 | |
| 1406 | 2775 | 2822 | 3128 | 2887 | |
| 1407 | 2775 | 2823 | 3128 | 2887 | |
| 1408 | 2776 | 2823 | 3129 | 2888 | |
| 1409 | 2776 | 2824 | 3129 | 2888 | |
| 1410 | 2777 | 2824 | 3130 | 2889 | |
| 1411 | 2777 | 2825 | 3130 | 2889 | |
| 1412 | 2778 | 2825 | 3131 | 2890 | |
| 1413 | 2779 | 2826 | 3131 | 2890 | |
| 1414 | 2779 | 2826 | 3132 | 2891 | |
| 1415 | 2965 | 2827 | 3133 | 2891 | |
| 1416 | 2965 | 2827 | 3133 | 2892 | |
| 1417 | 2966 | 2828 | 3134 | 2893 | |
| 1418 | 2966 | 2828 | 3134 | 2893 | |
| 1419 | 2967 | 2829 | 3135 | 2894 | |
| 1420 | 2968 | 2829 | 3135 | 2894 | |
| 1421 | 2968 | 2830 | 3136 | 2895 | |
| 1422 | 2969 | 2830 | 3136 | 2895 | |
| 1423 | 2969 | 2831 | 6181 | 2896 | |
| 1424 | 2970 | 2831 | 6183 | 2896 | |
| 1425 | 2970 | 2832 | 6284 | 2897 | |
| 1426 | 2971 | 2832 | 6285 | 2897 | |
| 1427 | 2971 | 2833 | 6760 | 2898 | |
| 1428 | 2972 | 2833 | 6761 | 2898 | |
| 1429 | 2972 | 2834 | 7036 | 2899 | |
| 1430 | 2973 | 2834 | 7038 | 2899 | |
| 1431 | 2974 | 2835 | 7158 | 2900 | |
| 1432 | 2974 | 2835 | 7160 | 2900 | |
| 1433 | 2975 | 2836 | 2610 | 2901 | |
| 1434 | 2976 | 2836 | 2610 | 2901 | |
| 1435 | 2976 | 2837 | 2611 | 2902 | |
| 1436 | 2977 | 2837 | 2611 | 2902 | |
| 1437 | 2977 | 2838 | 2612 | 2903 | |
| 1439 | 2978 | 2838 | 2612 | 2903 | |
| 1440 | 2978 | 2839 | 2613 | 2904 | |
| 1441 | 2979 | 2839 | 2613 | 2904 | |
| 1442 | 2979 | 2840 | 2614 | 2905 | |
| 1443 | 2980 | 2840 | 2614 | 2905 | |
| 1444 | 2980 | 2841 | 2615 | 2906 | |
| 1445 | 2981 | 2841 | 2615 | 2906 | |
| 1446 | 2981 | 2842 | 2616 | 2907 | |
| 1447 | 2982 | 2842 | 2616 | 2907 | |
| 6001 | 2982 | 2843 | 2617 | 2908 | |
| 6030 | 2983 | 2843 | 2617 | 2908 | |
| 6078 | 2983 | 2844 | 2618 | 2909 | |
| 6108 | 2984 | 2844 | 2618 | 2909 | |
| 6130 | 2985 | 2845 | 2619 | 2910 | |
| 6165 | 2985 | 2845 | 2619 | 2910 | |
| 6265 | 2986 | 2846 | 2620 | 2911 | |
| 6275 | 2987 | 2846 | 2620 | 2911 | |
| 6305 | 2987 | 2847 | 2621 | 2912 | |
| 6329 | 2988 | 2847 | 2621 | 2912 | |
| 6370 | 2988 | 2848 | 2622 | 2913 | |
| 6385 | 2989 | 2848 | 2622 | 2913 | |
| 6404 | 2989 | 2849 | 2623 | 2914 | |
| 6531 | 2990 | 2849 | 2623 | 2914 | |
| 6585 | 2990 | 2850 | 2624 | 2915 | |
| 6622 | 2991 | 2850 | 2624 | 2915 | |
| 6652 | 2991 | 2851 | 2625 | 2916 | |
| 6733 | 2992 | 2851 | 2625 | 2916 | |
| 6756 | 2992 | 2852 | 2626 | 2917 | |
| 6765 | 2993 | 2852 | 2626 | 2917 | |
| 6798 | 2993 | 2853 | 2627 | 2918 | |
| 6824 | 2994 | 2853 | 2627 | 2919 | |
| 6972 | 2994 | 2854 | 2628 | 2919 | |
| 7046 | 2995 | 2854 | 2628 | 2920 | |
| 7054 | 2995 | 2855 | 2629 | 2920 | |
| 7069 | 2996 | 2855 | 2629 | 2921 | |
| 7080 | 2996 | 2856 | 2630 | 2921 | |
| 7105 | 2997 | 2856 | 2630 | 2922 | |
| 7123 | 2998 | 2857 | 2631 | 2922 | |
| 7143 | 2998 | 2857 | 2631 | 2923 | |
| 7152 | 2999 | 2858 | 2632 | 2923 | |
| 7204 | 2999 | 2858 | 2632 | 2924 | |
| 7320 | 3001 | 2859 | 2633 | 2924 | |
| 7351 | 3001 | 2859 | 2633 | 2925 | |
| 7381 | 3002 | 2860 | 2634 | 2925 | |
| 7403 | 3002 | 2860 | 2634 | 2926 | |
| 7438 | 3003 | 2861 | 2635 | 2926 | |
| 7488 | 3003 | 2861 | 2635 | 2927 | |
| 7500 | 3004 | 3035 | 2636 | 2927 | |
| 7526 | 3004 | 3036 | 2636 | 2928 | |
| 7588 | 3005 | 3036 | 2637 | 2928 | |
| 7612 | 3005 | 3037 | 2637 | 2929 | |
| 7627 | 3006 | 3037 | 2638 | 2929 | |

### Systems

In an aspect, the disclosure relates to a system comprising nucleic acid molecule encoding a gene modifying polypeptide (e.g., as described herein) and a template nucleic acid (e.g., a template RNA, e.g., as described herein). In certain embodiments, the nucleic acid molecule encoding the gene modifying polypeptide comprises one or more silent mutations in the coding region (e.g., in the sequence encoding the RT domain) relative to a nucleic acid molecule as described herein. In certain embodiments, the system further comprises a gRNA (e.g., a gRNA that binds to a polypeptide that induces a nick, e.g., in the opposite strand of the target DNA bound by the gene modifying polypeptide).

In certain embodiments, the nucleic acid molecule encoding the gene modifying polypeptide encodes a polypeptide having an amino acid sequence selected from SEQ ID NOs: 1-7743, or an amino acid sequence having at least 70%, 75%, 80%, 85%, 90%, 95%, or 99% identity thereto. In certain embodiments, the nucleic acid molecule encoding the gene modifying polypeptide encodes a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the nucleic acid molecule encoding the gene modifying polypeptide encodes a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the nucleic acid molecule encoding the gene modifying polypeptide encodes a polypeptide as listed in any of Tables T1 or T2, or an amino acid sequence having at least 70%, 75%, 80%, 85%, 90%, 95%, or 99% identity thereto.

In certain embodiments, the nucleic acid molecule encoding the gene modifying polypeptide comprises a sequence encoding a portion of an amino acid sequence selected from SEQ ID NOs: 1-7743, wherein the portion comprises a linker and RT domain, or an amino acid sequence having at least 70%, 75%, 80%, 85%, 90%, 95%, or 99% identity to said portion. In certain embodiments, the nucleic acid molecule encoding the gene modifying polypeptide comprises a sequence encoding a portion of an amino acid sequence selected from SEQ ID NOs: 6001-7743, wherein the portion comprises a linker and RT domain, or an amino acid sequence having at least 70%, 75%, 80%, 85%, 90%, 95%, or 99% identity to said portion. In certain embodiments, the nucleic acid molecule encoding the gene modifying polypeptide comprises a sequence encoding a portion of an amino acid sequence selected from SEQ ID NOs: 4501-4541, wherein the portion comprises a linker and RT domain, or an amino acid sequence having at least 70%, 75%, 80%, 85%, 90%, 95%, or 99% identity to said portion. In certain embodiments, the nucleic acid molecule encoding the gene modifying polypeptide comprises a sequence encoding a portion of a polypeptide listed in any of Tables T1 or T2, wherein the portion comprises a linker and RT domain, or an amino acid sequence having at least 70%, 75%, 80%, 85%, 90%, 95%, or 99% identity to said portion.

In certain embodiments, the nucleic acid molecule encoding the gene modifying polypeptide comprises a sequence encoding the linker of an amino acid sequence selected from SEQ ID NOs: 1-7743, or an amino acid sequence having at least 70%, 75%, 80%, 85%, 90%, 95%, or 99% identity thereto. In certain embodiments, the nucleic acid molecule encoding the gene modifying polypeptide comprises a sequence encoding the linker of a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the nucleic acid molecule encoding the gene modifying polypeptide comprises a sequence encoding the linker of a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the nucleic acid molecule encoding the gene modifying polypeptide comprises a sequence encoding the linker of a polypeptide as listed in any of Tables T1 or T2, or an amino acid sequence having at least 70%, 75%, 80%, 85%, 90%, 95%, or 99% identity thereto.

In certain embodiments, the nucleic acid molecule encoding the gene modifying polypeptide comprises a sequence encoding the RT domain of an amino acid sequence selected from SEQ ID NOs: 1-7743, or an amino acid sequence having at least 70%, 75%, 80%, 85%, 90%, 95%, or 99% identity thereto. In certain embodiments, the nucleic acid molecule encoding the gene modifying polypeptide comprises a sequence encoding the RT domain of a polypeptide having an amino acid sequence selected from SEQ ID NOs: 6001- 7743, or an amino acid sequence having at least 70%, 75%, 80%, 85%, 90%, 95%, or 99% identity thereto. In certain embodiments, the nucleic acid molecule encoding the gene modifying polypeptide comprises a sequence encoding the RT domain of a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the nucleic acid molecule encoding the gene modifying polypeptide comprises a sequence encoding the RT domain of a polypeptide as listed in any of Tables T1 or T2, or an amino acid sequence having at least 70%, 75%, 80%, 85%, 90%, 95%, or 99% identity thereto.

In an aspect, the disclosure relates to a system comprising a gene modifying polypeptide (e.g., as described herein) and a template nucleic acid (e.g., a template RNA, e.g., as described herein).

In certain embodiments, the gene modifying polypeptide comprises a polypeptide having an amino acid sequence selected from SEQ ID NOs: 1-7743, or an amino acid sequence having at least 70%, 75%, 80%, 85%, 90%, 95%, or 99% identity thereto. In certain embodiments, the gene modifying polypeptide comprises a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the gene modifying polypeptide comprises a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the gene modifying polypeptide comprises a polypeptide as listed in any of Tables T1 or T2, or an amino acid sequence having at least 70%, 75%, 80%, 85%, 90%, 95%, or 99% identity thereto.

In certain embodiments, the gene modifying polypeptide comprises a portion of an amino acid sequence selected from SEQ ID NOs: 1-7743, wherein the portion comprises a linker and RT domain, or an amino acid sequence having at least 70%, 75%, 80%, 85%, 90%, 95%, or 99% identity to said portion. In certain embodiments, the gene modifying polypeptide comprises a portion of an amino acid sequence selected from SEQ ID NOs: 6001-7743, wherein the portion comprises a linker and RT domain, or an amino acid sequence having at least 70%, 75%, 80%, 85%, 90%, 95%, or 99% identity to said portion. In certain embodiments, the gene modifying polypeptide comprises a portion of an amino acid sequence selected from SEQ ID NOs: 4501-4541, wherein the portion comprises a linker and RT domain, or an amino acid sequence having at least 70%, 75%, 80%, 85%, 90%, 95%, or 99% identity to said portion. In certain embodiments, the gene modifying polypeptide comprises a portion of a polypeptide listed in any of Tables T1 or T2, wherein the portion comprises a linker and RT domain, or an amino acid sequence having at least 70%, 75%, 80%, 85%, 90%, 95%, or 99% identity to said portion.

In certain embodiments, the gene modifying polypeptide comprises the linker of an amino acid sequence selected from SEQ ID NOs: 1-7743, or an amino acid sequence having at least 70%, 75%, 80%, 85%, 90%, 95%, or 99% identity thereto. In certain embodiments, the gene modifying polypeptide comprises a sequence encoding the linker of a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the gene modifying polypeptide comprises a sequence encoding the linker of a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the gene modifying polypeptide comprises the linker of a polypeptide as listed in any of Tables T1 or T2, or an amino acid sequence having at least 70%, 75%, 80%, 85%, 90%, 95%, or 99% identity thereto.

In certain embodiments, the gene modifying polypeptide comprises the RT domain of an amino acid sequence selected from SEQ ID NOs: 1-7743, or an amino acid sequence having at least 70%, 75%, 80%, 85%, 90%, 95%, or 99% identity thereto. In certain embodiments, the gene modifying polypeptide comprises a sequence encoding the RT domain of a polypeptide having an amino acid sequence selected from SEQ ID NOs: 6001-7743, or an amino acid sequence having at least 70%, 75%, 80%, 85%, 90%, 95%, or 99% identity thereto. In certain embodiments, the gene modifying polypeptide comprises a sequence encoding the RT domain of a polypeptide having an amino acid sequence selected from SEQ ID NOs: 4501-4541, or an amino acid sequence having at least 70%, 75%, 80%, 85%, 90%, 95%, or 99% identity thereto. In certain embodiments, the gene modifying polypeptide comprises the RT domain of a polypeptide as listed in any of Tables T1 or T2, or an amino acid sequence having at least 70%, 75%, 80%, 85%, 90%, 95%, or 99% identity thereto.

### Systems for Modifying DNA

Also provided herein are systems for modifying DNA. The gene modifying systems can, for example, comprise (a) a gene modifying polypeptide or a nucleic acid molecule encoding the gene modifying polypeptide, wherein the gene modifying polypeptide comprise (i) a reverse transcriptase (RT) domain, and either an endonuclease domain that contains DNA binding functionality or an endonuclease domain and separate DNA binding domain; and (b) a template RNA.

Thus, provided herein are systems for modifying DNA. The systems can, for example, comprise (a) an artificial ribonucleic acid (RNA) molecule for enhancing expression of a polypeptide comprising a reverse transcriptase (RT) domain and optionally an endonuclease domain, the artificial RNA molecule comprising: (1) a nucleotide sequence encoding the polypeptide; (2) at least one of: (i) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269 and 8281; and/or (ii) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or (3) a poly(A) tail for enhancing expression of the polypeptide, wherein the poly(A) tail comprises a nucleic acid sequence selected from the group consisting of SEQ ID NOs: 8282-8314; and (b) a template RNA (or DNA encoding the template RNA) comprising (e.g., from 5′ to 3′): (i) optionally a sequence that binds a target site in the DNA (e.g., a second strand of a site in a target genome); (ii) a sequence that binds the polypeptide; (iii) a heterologous object sequence; and (iv) optionally a 3′ target homology domain. The heterologous object sequence can, for example, comprise an alteration relative to a corresponding original sequence (e.g., a wild-type sequence), wherein the alteration improves the speed, fidelity, or speed and fidelity of target-primed reverse transcription by the reverse transcriptase.

In certain embodiments, the systems can, for example, comprise (a) an artificial ribonucleic acid (RNA) molecule for enhancing expression of a polypeptide comprising a reverse transcriptase (RT) domain and optionally an endonuclease domain, the artificial nucleic acid molecule comprising: (1) a nucleotide sequence encoding the polypeptide; (2) at least one of: (i) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269 and 8281; and/or (ii) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or (3) a poly(A) tail for enhancing expression of the polypeptide, wherein the poly(A) tail comprises a nucleic acid sequence selected from the group consisting of SEQ ID NOs: 8282-8314; and (b) a template RNA (or DNA encoding the template RNA) comprising (e.g., from 5′ to 3′): (i) optionally a sequence that binds a target site in the DNA (e.g., a second strand of a site in a target genome), (ii) a sequence that binds the polypeptide, (iii) a heterologous object sequence, and (iv) optionally a 3′ target homology domain. Preferably, the heterologous object sequence has one or both of the following characteristics: i) does not comprise self-complementary sequences, e.g., that form hairpin structures, e.g., under stringent conditions, or if a self-complementary sequence is present, it has one, two, or all of the following characteristics (1) each self-complementary sequence is no more than 10, 9, 8, 7, 6, 5, 4, or 3 nucleotides in length; (2) the self-complementary sequence forms a hairpin comprising arms of no longer than 10, 9, 8, 7, 6, 5, 4, or 3 nucleotides in length, or (3) the self-complementary sequence comprises at least 1, 2, 3, 4, or 5 positions of non-complementarity (e.g., mismatches or bulges) with its partner sequence, and (4) does not comprise a repetitive sequence (e.g., a single-, di-, or tri-nucleotide repetitive sequence) or if a repetitive sequence is present it is of no more than 12, 11, 10, 9, 8, 7, or 6 nucleotides in length.

### Template Nucleic Acid or RNA

The gene modifying systems described herein can modify a host target DNA site by using a template nucleic acid. In some embodiments, the gene modifying systems described herein transcribe an RNA sequence template into host target DNA sites by target-primed reverse transcription (TPRT). By modifying DNA sequence(s) via reverse transcription of the RNA sequence template directly into the host genome, the gene modifying system can insert an object sequence into a target genome without the need for exogenous DNA sequences to be introduced into the host cell (unlike, for example, CRISPR systems), as well as eliminate an exogenous DNA insertion step. The gene modifying system can also delete a sequence from the target genome or introduce a substitution using an object sequence. Therefore, the gene modifying system provides a platform for the use of customized RNA sequence templates containing object sequences, e.g., sequences comprising heterologous gene coding and/or function information.

In some embodiments, the template nucleic acid comprises one or more sequence (e.g., 2 sequences) that binds the gene modifying polypeptide.

In some embodiments, the template nucleic acid comprises RNA. In some embodiments, the template nucleic acid comprises DNA (e.g., single stranded or double stranded DNA).

In some embodiments, the template nucleic acid comprises one or more (e.g., 2) homology domains that have homology to the target sequence. In some embodiments, the homology domains are about 10-20, 20-50, or 50-100 nucleotides in length.

In some embodiments, a template RNA can comprise a gRNA sequence, e.g., to direct the gene modifying polypeptide to a target site of interest. In some embodiments, a template RNA comprises (e.g., from 5′ to 3′) (i) optionally a gRNA spacer that binds a target site (e.g., a second strand of a site in a target genome), (ii) optionally a gRNA scaffold that binds a polypeptide described herein (e.g., a gene modifying polypeptide or a Cas polypeptide), (iii) a heterologous object sequence comprising a mutation region (optionally the heterologous object sequence comprises, from 5′ to 3′, a first homology region, a mutation region, and a second homology region), and (iv) a primer binding site (PBS) sequence comprising a 3′ target homology domain.

The template nucleic acid (e.g., template RNA) component of a genome editing system described herein typically is able to bind the gene modifying polypeptide of the system. In some embodiments the template nucleic acid (e.g., template RNA) has a 3′ region that is capable of binding a gene modifying polypeptide. The binding region, e.g., 3′ region, may be a structured RNA region, e.g., having at least 1, 2 or 3 hairpin loops, capable of binding the gene modifying polypeptide of the system. The binding region may associate the template nucleic acid (e.g., template RNA) with any of the polypeptide modules. In some embodiments, the binding region of the template nucleic acid (e.g., template RNA) may associate with an RNA-binding domain in the polypeptide. In some embodiments, the binding region of the template nucleic acid (e.g., template RNA) may associate with the reverse transcription domain of the gene modifying polypeptide (e.g., specifically bind to the RT domain). In some embodiments, the template nucleic acid (e.g., template RNA) may associate with the DNA binding domain of the polypeptide, e.g., a gRNA associating with a Cas9-derived DNA binding domain. In some embodiments, the binding region may also provide DNA target recognition, e.g., a gRNA hybridizing to the target DNA sequence and binding the polypeptide, e.g., a Cas9 domain. In some embodiments, the template nucleic acid (e.g., template RNA) may associate with multiple components of the polypeptide, e.g., DNA binding domain and reverse transcription domain.

In some embodiments, the template nucleic acid is a template RNA. In some embodiments, the template RNA comprises one or more modified nucleotides. For example, in some embodiments, the template RNA comprises one or more deoxyribonucleotides. In some embodiments, regions of the template RNA are replaced by DNA nucleotides, e.g., to enhance stability of the molecule. For example, the 3′ end of the template may comprise DNA nucleotides, while the rest of the template comprises RNA nucleotides that can be reverse transcribed. For instance, in some embodiments, the heterologous object sequence is primarily or wholly made up of RNA nucleotides (e.g., at least 90%, 95%, 98%, or 99% RNA nucleotides). In some embodiments, the PBS sequence is primarily or wholly made up of DNA nucleotides (e.g., at least 90%, 95%, 98%, or 99% DNA nucleotides). In other embodiments, the heterologous object sequence for writing into the genome may comprise DNA nucleotides. In some embodiments, the DNA nucleotides in the template are copied into the genome by a domain capable of DNA-dependent DNA polymerase activity. In some embodiments, the DNA-dependent DNA polymerase activity is provided by a DNA polymerase domain in the polypeptide. In some embodiments, the DNA-dependent DNA polymerase activity is provided by a reverse transcriptase domain that is also capable of DNA-dependent DNA polymerization, e.g., second strand synthesis. In some embodiments, the template molecule is composed of only DNA nucleotides.

In some embodiments, a system described herein comprises two nucleic acids which together comprise the sequences of a template RNA described herein. In some embodiments, the two nucleic acids are associated with each other non-covalently, e.g., directly associated with each other (e.g., via base pairing), or indirectly associated as part of a complex comprising one or more additional molecule.

A template RNA described herein may comprise, from 5′ to 3′: (1) a gRNA spacer; (2) a gRNA scaffold; (3) heterologous object sequence (4) a primer binding site (PBS) sequence.

As described herein a gRNA spacer can direct the gene modifying system to a target nucleic acid, and a gRNA scaffold can promote the association of the template RNA with the Cas domain of the gene modifying polypeptide, thus allowing for the editing of the target sequence. In certain embodiments, a gRNA that comprises a gRNA spacer and a gRNA scaffold, but not a heterologous object sequence or a PBS sequence can, for example, be used to induce second strand nicking.

As described herein a heterologous object sequence can be used as a template for the gene modifying polypeptide for reverse transcription to write a desired sequence into the target nucleic acid. In some embodiments, the heterologous object sequence comprises, from 5′ to 3′, a post-edit homology region, the mutation region, and a pre-edit homology region. Without wishing to be bound by theory, an RT performing reverse transcription on the template RNA first reverse transcribes the pre-edit homology region, then the mutation region, and then the post-edit homology region, thereby creating a DNA strand comprising the desired mutation with a homology region on either side.

As described herein, a template nucleic acid can, for example, comprise a PBS sequence. In some embodiments, a PBS sequence is disposed 3′ of the heterologous object sequence and is complementary to a sequence adjacent to a site to be modified by a system described herein, or comprises no more than 1, 2, 3, 4, or 5 mismatches to a sequence complementary to the sequence adjacent to a site to be modified by the system/gene modifying polypeptide. In some embodiments, the PBS sequence binds within 1, 2, 3, 4, 5, 6, 7, 8, 9, or 10 nucleotides of a nick site in the target nucleic acid molecule. In some embodiments, binding of the PBS sequence to the target nucleic acid molecule permits initiation of target-primed reverse transcription (TPRT), e.g., with the 3′ homology domain acting as a primer for TPRT. In some embodiments, the PBS sequence is 3-5, 5-10, 10-30, 10-25, 10-20, 10-19, 10-18, 10-17, 10-16, 10-15, 10-14, 10-13, 10-12, 10-11, 11-30, 11-25, 11-20, 11-19, 11-18, 11-17, 11-16, 11-15, 11-14, 11-13, 11-12, 12-30, 12-25, 12-20, 12-19, 12-18, 12-17, 12-16, 12-15, 12-14, 12-13, 13-30, 13-25, 13-20, 13-19, 13-18, 13-17, 13-16, 13-15, 13-14, 14-30, 14-25, 14-20, 14-19, 14-18, 14-17, 14-16, 14-15, 15-30, 15-25, 15-20, 15-19, 15-18, 15-17, 15-16, 16-30, 16-25, 16-20, 16-19, 16-18, 16-17, 17-30, 17-25, 17-20, 17-19, 17-18, 18-30, 18-25, 18-20, 18-19, 19-30, 19-25, 19-20, 20-30, 20-25, or 25-30 nucleotides in length, e.g., 10-17, 12-16, or 12-14 nucleotides in length. In some embodiments, the PBS sequence is 5-20, 8-16, 8-14, 8-13, 9-13, 9-12, or 10-12 nucleotides in length, e.g., 9-12 nucleotides in length.

Template nucleic acid and template RNA is described in WO2021/248102, incorporated herein by reference in its entirety.

In certain embodiments, the template RNA further comprises a reverse transcriptase (RT) terminator sequence situated between the heterologous object sequence and either (i) or (ii).

In certain embodiments, the heterologous object sequence encodes a target polypeptide or portion thereof or comprises a sequence that is the reverse complement of a sequence encoding the target polypeptide or portion thereof.

In certain embodiments, the polypeptide comprises the reverse transcriptase domain and the endonuclease domain, and the endonuclease domain is a Cas9 domain, and the template RNA comprises (i) a gRNA spacer that is complementary to a first portion of a target gene, and optionally comprises one or more consecutive nucleotides starting with the 3′ end of the flanking nucleotides of the gRNA spacer; (ii) a gRNA scaffold that binds to the Cas9 domain; (iii) a heterologous object sequence comprising a mutation region to introduce a mutation into (e.g., to correct a mutation in) a second portion of the target gene (wherein optionally the heterologous sequence comprises, from 5′ to 3′ a post-edit homology region, a mutation region, and a pre-edit homology region), and (iv) a primer binding site (PBS) sequence comprising at least 5, 6, 7, or 8 bases with 100% identity to a third portion of the target gene.

Template RNA sequences are also provided in Tables 1A-1D, 5A-5F, 8A-8D, E3, E3A, BB, E5, E5A, E6, and E6A of WO2023039435, which is incorporated herein by reference in its entirety.

In certain embodiments, the target gene is a human PAH gene, and the template RNA comprises (i) a gRNA spacer that is complementary to a first portion of the human PAH gene, wherein the gRNA spacer has a sequence comprising the core nucleotides of a gRNA spacer sequence, preferably of Table 1A, Table 1B, Table 1C, or Table 1D in WO2023039435, which is herein incorporated by reference in its entirety, and optionally comprises one or more consecutive nucleotides starting with the 3′ end of the flanking nucleotides of the gRNA spacer, or wherein the gRNA spacer has a sequence of a spacer chosen from Tables 5A-5F, 8A-8D, E3, E3A, BB, E5, E5A, E6, or E6A in WO2023039435, which is herein incorporated by reference in its entirety; (ii) a gRNA scaffold that binds to the Cas9 domain; (iii) a heterologous object sequence comprising a mutation region to introduce a mutation into (e.g., to correct a mutation in) a second portion of the human PAH gene (wherein optionally the heterologous object sequence comprises, from 5′ to 3′, a post-edit homology region, a mutation region, and a pre-edit homology region); and (iv) a primer binding site (PBS) sequence comprising at least 5, 6, 7, or 8 bases with 100% identity to a third portion of the human PAH gene.

In certain embodiments, the template RNA consists of the sequence of SEQ ID NO: 8258 (RNACS7570); SEQ ID NO: 8320 (RNACS229); SEQ ID NO: 8321 (RNACS1515), or SEQ ID NO: 8372.

In certain embodiments, the target site is in a human genome.

### Therapeutic Applications

By integrating coding genes into a RNA sequence template, the system can address therapeutic needs, for example, by providing expression of a therapeutic transgene in individuals with loss-of-function mutations, by replacing gain-of-function mutations with normal transgenes, by providing regulatory sequences to eliminate gain-of-function mutation expression, and/or by controlling the expression of operably linked genes, transgenes and systems thereof. In certain embodiments, the RNA sequence template encodes a promotor region specific to the therapeutic needs of the host cell, for example a tissue specific promotor or enhancer. In still other embodiments, a promotor can be operably linked to a coding sequence.

In some embodiments, a system as described herein can be used to make an insertion, deletion, substitution, or combination thereof in a cell, tissue, or subject. In some embodiments, an insertion, deletion, substitution, or combination thereof, increases or decreases expression (e.g., transcription or translation) of a gene. In some embodiments, an insertion, deletion, substitution, or combination thereof, increases or decreases expression (e.g., transcription or translation) of a gene by altering, adding, or deleting sequences in a promoter or enhancer, e.g., sequences that bind transcription factors. In some embodiments, an insertion, deletion, substitution, or combination thereof alters translation of a gene (e.g., alters an amino acid sequence), inserts or deletes a start or stop codon, alters or fixes the translation frame of a gene.

In some embodiments, an insertion, deletion, substitution, or combination thereof alters splicing of a gene, e.g., by inserting, deleting, or altering a splice acceptor or donor site. In some embodiments, an insertion, deletion, substitution, or combination thereof alters transcript or protein half-life. In some embodiments, an insertion, deletion, substitution, or combination thereof alters protein localization in the cell (e.g., from the cytoplasm to a mitochondria, from the cytoplasm into the extracellular space (e.g. adds a secretion tag)). In some embodiments, an insertion, deletion, substitution, or combination thereof alters (e.g., improves) protein folding (e.g., to prevent accumulation of misfolded proteins). In some embodiments, an insertion, deletion, substitution, or combination thereof, alters, increases, decreases the activity of a gene, e.g., a protein encoded by the gene.

The disclosure is directed, in part, to a method of modifying a target site in genomic DNA in a cell. In some embodiments, the method comprises contacting the cell with a system, template RNA, virus, viral-like particle, or virosome, or LNP described herein, or DNA encoding the same, thereby modifying the target site in genomic DNA in a cell.

Thus, provided are methods for modifying a target site in genomic DNA in a cell. The methods comprise contacting the cell with the system of the invention or one or more RNAs encoding the system of the invention, thereby modifying the target site in the genomic DNA in a cell. In certain embodiments, the cell is a T cell (e.g., a primary T cell).

The disclosure is directed, in part, to a method for treating a subject having a disease or condition associated with a genetic defect. In some embodiments, the method comprises administering to the subject a system, template RNA, virus, viral-like particle, or virosome, or LNP described herein, or DNA encoding the same, thereby treating the subject having a disease or condition associated with a genetic defect. In some embodiments, the disease or condition associated with a genetic defect is an indication listed in any of Tables 9-12 of International Application Publication WO2021/178720, which is herein incorporated by reference in its entirety including said tables, and/or wherein the genetic defect is a defect in a gene listed in any of said Tables 9-12 therein. In some embodiments, the subject is a human subject.

Thus, provided are methods for treating a subject having a disease or condition associated with a genetic defect. The methods comprise administering to the subject the system of the invention, thereby treating the subject having a disease or condition associated with a genetic defect.

Accordingly, provided herein are methods for treating phenylketonuria (PKU) or hyperphenylalaninemia (e.g., mild or severe hyperphenylalaninemia) in a subject in need thereof. In some embodiments, treatment results in amelioration of one or more symptoms associated with PKU or hyperphenylalaninemia, as indicated in WO2023039435, which is incorporated by reference herein in its entirety.

In some embodiments, treatment with a gene modifying system described herein results in one or more of (a) an increase in phenylalanine hydroxylase (PAH) activity, efficiency, and/or function; (b) a decrease in the concentration of phenylalanine in the blood and/or cerebrospinal fluid; (c) increase in the concentration of tyrosine in the blood; (d) a restoration of normal synthesis of dopamine, norepinephrine, and/or melanin; (e) a reduction in ureagenesis; and/or (f) an improvement in protein retention and/or Phe utilization as compared to a subject having PKU that has not been treated with a gene modifying system described herein.

### Administration and Delivery

The compositions and systems described herein may be used in vitro or in vivo. In some embodiments the system or components of the system are delivered to cells (e.g., mammalian cells, e.g., human cells), e.g., in vitro or in vivo. In some embodiments, the cells are eukaryotic cells, e.g., cells of a multicellular organism, e.g., an animal, e.g., a mammal (e.g., human, swine, bovine), a bird (e.g., poultry, such as chicken, turkey, or duck), or a fish. In some embodiments, the cells are non-human animal cells (e.g., a laboratory animal, a livestock animal, or a companion animal). In some embodiments, the cell is a stem cell (e.g., a hematopoietic stem cell), a fibroblast, or a T cell. In some embodiments, the cell is an immune cell, e.g., a T cell (e.g., a Treg, CD4, CD8, γδ, or memory T cell), B cell (e.g., memory B cell or plasma cell), or NK cell. In some embodiments, the cell is a non-dividing cell, e.g., a non-dividing fibroblast or non-dividing T cell.

In one embodiment the system and/or components of the system are delivered as nucleic acid. For example, the gene modifying polypeptide may be delivered in the form of a DNA or RNA encoding the polypeptide, and the template RNA may be delivered in the form of RNA or its complementary DNA to be transcribed into RNA. In some embodiments the system or components of the system are delivered on 1, 2, 3, 4, or more distinct nucleic acid molecules. In some embodiments the system or components of the system are delivered as a combination of DNA and RNA. In some embodiments the system or components of the system are delivered as a combination of DNA and protein. In some embodiments the system or components of the system are delivered as a combination of RNA and protein. In some embodiments the gene modifying polypeptide is delivered as a protein.

In some embodiments the system or components of the system are delivered to cells, e.g., mammalian cells or human cells, using a vector. The vector may be, e.g., a plasmid or a virus. In some embodiments, delivery is in vivo, in vitro, ex vivo, or in situ. In some embodiments the virus is an adeno associated virus (AAV), a lentivirus, or an adenovirus. In some embodiments the system or components of the system are delivered to cells with a viral-like particle or a virosome. In some embodiments the delivery uses more than one virus, viral-like particle or virosome.

In one embodiment, the compositions and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

A variety of nanoparticles can be used for delivery, such as a liposome, a lipid nanoparticle, a cationic lipid nanoparticle, an ionizable lipid nanoparticle, a polymeric nanoparticle, a gold nanoparticle, a dendrimer, a cyclodextrin nanoparticle, a micelle, or a combination of the foregoing.

The methods and systems provided by the invention, may employ any suitable carrier or delivery modality, including, in certain embodiments, lipid nanoparticles (LNPs). Any LNPs known in the art may be used. LNPs are described in WO2021/178898, incorporated herein by reference in its entirety.

### Kits

The disclosure is also directed, in part, to kits comprising (a) a system, template nucleic acid (e.g., template RNA), a reaction mixture, a DNA molecule, an RNA molecule, or a pharmaceutical composition as described herein, and (b) instructions for using the system, template nucleic acid (e.g., template RNA), reaction mixture, DNA molecule, RNA molecule, or the pharmaceutical composition described herein. In some embodiments, a kit further comprises a cell (e.g., a cell from a cell line or a cell from a subject, e.g., a human cell) or DNA (e.g., genomic DNA or a vector) comprising a target site (e.g., a target site that is the target of the system or template RNA).

### EMBODIMENTS

The application includes, but is not limited to, the following numbered embodiments:

Embodiment 1 is an artificial ribonucleic acid (RNA) molecule for enhancing expression of a polypeptide comprising a reverse transcriptase (RT) domain and optionally an endonuclease domain, the RNA molecule comprising:
(1) a nucleotide sequence encoding the polypeptide;
(2) at least one of:
   (a) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269, and 8281; and/or
   (b) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or
(3) a poly(A) tail for enhancing expression of the polypeptide, wherein the poly(A) tail comprises a nucleic acid sequence selected from the group consisting of SEQ ID NOs: 8282-8314.

Embodiment 1a is an artificial ribonucleic acid (RNA) molecule for enhancing expression of a polypeptide comprising a reverse transcriptase (RT) domain and optionally an endonuclease domain, the RNA molecule comprising:
(1) a nucleotide sequence encoding the polypeptide;
(2) at least one of:
   (a) a 3′-untranslated region (3′UTR) element for enhancing expression of the polypeptide, wherein the 3′UTR element consists of a nucleic acid sequence selected from the group consisting of SEQ ID NOs: 8201-8211, 8256, 8264-8269, and 8281; and/or
   (b) a 5′-untranslated region (5′UTR) element for enhancing expression of the polypeptide, wherein the 5′UTR element consists of a nucleic acid sequence selected form the group consisting of SEQ ID NOs: 8212-8247 and 8259-8263; and/or
(3) a poly(A) tail for enhancing expression of the polypeptide, wherein the poly(A) tail comprises a nucleic acid sequence selected from the group consisting of SEQ ID NOs: 8282-8314.

Embodiment 1b is an artificial ribonucleic acid (RNA) molecule for enhancing expression of a polypeptide comprising a reverse transcriptase (RT) domain and optionally an endonuclease domain, the RNA molecule comprising a nucleotide sequence encoding the polypeptide; and at least one of:
(a) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269, and 8281; and/or
(b) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263.

Embodiment 1c is an artificial ribonucleic acid (RNA) molecule for enhancing expression of a polypeptide comprising a reverse transcriptase (RT) domain and optionally an endonuclease domain, the RNA molecule comprising a nucleotide sequence encoding the polypeptide; and at least one of:
(a) a 3′-untranslated region (3′UTR) element for enhancing expression of the polypeptide, wherein the 3′UTR element consists of a nucleic acid sequence selected from the group consisting of SEQ ID NOs: 8201-8211, 8256, 8264-8269, and 8281; and/or
(b) a 5′-untranslated region (5′UTR) element for enhancing expression of the polypeptide, wherein the 5′UTR element consists of a nucleic acid sequence selected form the group consisting of SEQ ID NOs: 8212-8247 and 8259-8263.

Embodiment 1d is an artificial ribonucleic acid (RNA) molecule for enhancing expression of a polypeptide comprising a reverse transcriptase (RT) domain and optionally an endonuclease domain, the RNA molecule comprising:
(a) a nucleotide sequence encoding the polypeptide; and
(b) a poly(A) tail for enhancing expression of the polypeptide, wherein the poly(A) tail comprises a nucleic acid sequence selected from the group consisting of SEQ ID NOs: 8201-8233.

Embodiment 1e is an artificial ribonucleic acid (RNA) molecule for enhancing expression of a polypeptide comprising a reverse transcriptase (RT) domain and optionally an endonuclease domain, the RNA molecule comprising:
(a) a nucleotide sequence encoding the polypeptide; and
(b) a poly(A) tail for enhancing expression of the polypeptide, wherein the poly(A) tail consists of a nucleic acid sequence selected from the group consisting of SEQ ID NOs: 8282-8314.

Embodiment 2 is the artificial RNA molecule of embodiment 1 or 1b, wherein the 3′UTR element comprises a nucleic acid sequence of SEQ ID NO: 8201, SEQ ID NO: 8204, SEQ ID NO: 8209, SEQ ID NO: 8211, SEQ ID NO: 8256, or SEQ ID NO: 8281.

Embodiment 2a is the artificial RNA molecule of embodiment 1a or 1c, wherein the 3′UTR element consists of a nucleic acid sequence of SEQ ID NO: 8201, SEQ ID NO: 8204, SEQ ID NO: 8209, SEQ ID NO: 8211, SEQ ID NO: 8256, or SEQ ID NO: 8281.

Embodiment 3 is the artificial RNA molecule of any one of embodiments 1, 1b, or 2, wherein the 5′UTR element comprises a nucleic acid sequence of SEQ ID NO: 8214, SEQ ID NO: 8230, SEQ ID NO: 8235, SEQ ID NO: 8236, SEQ ID NO: 8243, SEQ ID NO: 8259, or SEQ ID NO: 8263.

Embodiment 3a is the artificial RNA molecule of any one of embodiments 1a, 1c, or 2a, wherein the 5′UTR element consists of a nucleic acid sequence of SEQ ID NO: 8214, SEQ ID NO: 8230, SEQ ID NO: 8235, SEQ ID NO: 8236, SEQ ID NO: 8243, SEQ ID NO: 8259, or SEQ ID NO: 8263.

Embodiment 4 is the artificial RNA molecule of any one of embodiments 1, 1b, 2, or 3, comprising the 3′UTR element and the 5′UTR element, wherein:
(1) the 3′UTR element comprises SEQ ID NO: 8209 and the 5′UTR element comprises SEQ ID NO: 8236;
(2) the 3′UTR element comprises SEQ ID NO: 8201 and the 5′UTR element comprises SEQ ID NO: 8236;
(3) the 3′UTR element comprises SEQ ID NO: 8209 and the 5′UTR element comprises SEQ ID NO: 8214;
(4) the 3′UTR element comprises SEQ ID NO: 8209 and the 5′UTR element comprises SEQ ID NO: 8243;
(5) the 3′UTR element comprises SEQ ID NO: 8281 and the 5′UTR element comprises SEQ ID NO: 8259; or
(6) the 3′UTR element comprises SEQ ID NO: 8281 and the 5′UTR element comprises SEQ ID NO: 8263.

Embodiment 4a is the artificial RNA molecule of any one of embodiments 1a, 1c, 2a, or 3a, comprising the 3′UTR element and the 5′UTR element, wherein:
(1) the 3′UTR element consists of SEQ ID NO: 8209 and the 5′UTR element consists of SEQ ID NO: 8236;
(2) the 3′UTR element consists of SEQ ID NO: 8201 and the 5′UTR element consists of SEQ ID NO: 8236;
(3) the 3′UTR element consists of SEQ ID NO: 8209 and the 5′UTR element consists of SEQ ID NO: 8214;
(4) the 3′UTR element consists of SEQ ID NO: 8209 and the 5′UTR element consists of SEQ ID NO: 8243;
(5) the 3′UTR element consists of SEQ ID NO: 8281 and the 5′UTR element consists of SEQ ID NO: 8259; or
(6) the 3′UTR element consists of SEQ ID NO: 8281 and the 5′UTR element consists of SEQ ID NO: 8263.

Embodiment 4b is the artificial RNA molecule of embodiment 4, comprising the 3′UTR element and the 5′UTR element, wherein the 3′UTR element comprises SEQ ID NO: 8201 and the 5′UTR element comprises SEQ ID NO: 8236.

Embodiment 4c is the artificial RNA molecule of embodiment 4, comprising the 3′UTR element and the 5′UTR element, wherein the 3′UTR element comprises SEQ ID NO: 8209 and the 5′UTR element comprises SEQ ID NO: 8214.

Embodiment 5 is the artificial RNA molecule of any one of embodiments 1, 1d, and 2-4c, wherein the poly(A) tail comprises a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

Embodiment 5a is the artificial RNA molecule of any one of embodiments 1, 1e, and 2-4c, wherein the poly(A) tail consists of a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

Embodiment 5b is the artificial RNA molecule of embodiment 5, wherein the poly(A) tail comprises the nucleic acid sequence of SEQ ID NO: 8306 or SEQ ID NO: 8307.

Embodiment 5c is the artificial RNA molecule of embodiment 5a, wherein the poly(A) tail consists of the nucleic acid sequence of SEQ ID NO: 8306 or SEQ ID NO: 8307.

Embodiment 5d is the artificial RNA molecule of any one of embodiments 1-3a, comprising the 3′UTR element, the 5′UTR element, and the poly(A) tail, wherein:
(1) the 3′UTR element comprises SEQ ID NO: 8209, the 5′UTR element comprises SEQ ID NO: 8236, and the poly(A) tail comprises SEQ ID NO: 8306 or SEQ ID NO: 8307;
(2) the 3′UTR element comprises SEQ ID NO: 8201, the 5′UTR element comprises SEQ ID NO: 8236, and the poly(A) tail comprises SEQ ID NO: 8306 or SEQ ID NO: 8307;
(3) the 3′UTR element comprises SEQ ID NO: 8209, the 5′UTR element comprises SEQ ID NO: 8214, and the poly(A) tail comprises SEQ ID NO: 8306 or SEQ ID NO: 8307;
(4) the 3′UTR element comprises SEQ ID NO: 8209, the 5′UTR element comprises SEQ ID NO: 8243, and the poly(A) tail comprises SEQ ID NO: 8306 or SEQ ID NO: 8307;
(5) the 3′UTR element comprises SEQ ID NO: 8281, the 5′UTR element comprises SEQ ID NO: 8259, and the poly(A) tail comprises SEQ ID NO: 8306 or SEQ ID NO: 8307; or
(6) the 3′UTR element comprises SEQ ID NO: 8281, the 5′UTR element comprises SEQ ID NO: 8263, and the poly(A) tail comprises SEQ ID NO: 8306 or SEQ ID NO: 8307.

Embodiment 6 is the artificial RNA molecule of any one of embodiments 1-5c, wherein the polypeptide comprises the reverse transcriptase domain and the endonuclease domain.

Embodiment 6a is the artificial RNA molecule of any one of embodiments 1-5c, wherein the polypeptide is a heterologous gene modifying polypeptide or a retrotransposon gene modifying polypeptide.

Embodiment 7 is the artificial RNA molecule of embodiment 6 or 6a, wherein the endonuclease domain is a nickase domain.

Embodiment 7a is the artificial RNA molecule of embodiment 7, wherein the nickase domain is a Cas9 domain, optionally, wherein the Cas9 domain is selected from a SpCas9 domain, a BlatCas9 domain, a Nme2 Cas9 domain, a PnpCas9 domain, a SauCas9 domain, a SauCas9-KKH domain, a SauriCas9 domain, a SauriCas9-KKH domain, a ScaCas9-Sc++ domain, a SpyCas9 domain, a SpyCas9-NG domain, a Spy Cas9-SpRY domain, or a St1Cas9 domain.

Embodiment 7b is the artificial RNA molecule of embodiment 7a, wherein the Cas9 domain comprises an N670A mutation, an N611A mutation, an N605A mutation, an N580A mutation, an N588A mutation, an N872A mutation, an N863A mutation, an N622A mutation, or an H840A mutation.

Embodiment 7c is the artificial RNA molecule of any one of embodiments 7-7b wherein the reverse transcriptase domain is selected from a retrovirus reverse transcriptase domain.

Embodiment 7d is the artificial RNA molecule of embodiment 7c, wherein the retrovirus reverse transcriptase domain is a gamma retrovirus-derived reverse transcriptase domain.

Embodiment 7e is the artificial RNA molecule of embodiment 7d, wherein the gamma retrovirus-derived reverse transcriptase domain comprises the amino acid sequence of a reverse-transcriptase domain sequence from a family selected from AVIRE, BAEVM, FFV, FLV, FOAMV, GALV, KORV, MLVAV, MLVBM, MLVCB, MLVFF, MLVMS, PERV, SFV1, SFV3L, WMSV, and XMRV6.

Embodiment 7f is the artificial RNA molecule of embodiment 7c or 7d, wherein the gamma retrovirus-derived reverse transcriptase domain is not derived from PERV.

Embodiment 7g is the artificial RNA molecule of any one of embodiments 7c-7f, wherein the reverse transcriptase domain comprises one, two, three, four, five, six or more mutations corresponding to the following mutations D200N, L603W, T330P, D524G, E562Q, D583N, P51L, S67R, T197A, H204R, E302K, F309N, W313F, L435G, N454K, H594Q, L671P, E69K, or D653N in the reverse transcriptase domain of murine leukemia virus reverse transcriptase.

Embodiment 7 h is the artificial RNA molecule of any one of embodiments 7-7g, wherein the polypeptide comprises the amino acid sequence of SEQ ID NO: 8257 or SEQ ID NO: 8371.

Embodiment 8 is an artificial ribonucleic acid (RNA) molecule comprising:
(1) at least one of:
   (a) a 3′-untranslated region (3′UTR) element comprising a nucleic acid sequence selected from the group consisting of SEQ ID NOs: 8201-8211; and/or
   (b) a 5′-untranslated region (5′UTR) element comprising a nucleic acid sequence selected form the group consisting of SEQ ID NOs: 8212-8247; and/or
(2) a poly(A) tail for enhancing expression of the polypeptide, wherein the poly(A) tail comprises a nucleic acid sequence selected from the group consisting of SEQ ID NOs: 8282-8314.

Embodiment 8a is an artificial ribonucleic acid (RNA) molecule consisting of:
(1) at least one of:
   (a) a 3′-untranslated region (3′UTR) element comprising a nucleic acid sequence selected from the group consisting of SEQ ID NOs: 8201-8211; and/or
   (b) a 5′-untranslated region (5′UTR) element comprising a nucleic acid sequence selected form the group consisting of SEQ ID NOs: 8212-8247; and/or
(2) a poly(A) tail for enhancing expression of the polypeptide, wherein the poly(A) tail consists of a nucleic acid sequence selected from the group consisting of SEQ ID NOs: 8282-8314.

Embodiment 8b is an artificial ribonucleic acid (RNA) molecule comprising:
(a) a 3′-untranslated region (3′UTR) element comprising a nucleic acid sequence selected from the group consisting of SEQ ID NOs: 8201-8211; and/or
(b) a 5′-untranslated region (5′UTR) element comprising a nucleic acid sequence selected form the group consisting of SEQ ID NOs: 8212-8247.

Embodiment 8c is an artificial ribonucleic acid (RNA) molecule consisting of:
(a) a 3′-untranslated region (3′UTR) element comprising a nucleic acid sequence selected from the group consisting of SEQ ID NOs: 8201-8211; and/or
(b) a 5′-untranslated region (5′UTR) element comprising a nucleic acid sequence selected form the group consisting of SEQ ID NOs: 8212-8247.

Embodiment 8d is an artificial ribonucleic acid (RNA) molecule comprising a poly(A) tail comprising a nucleic acid sequence selected from the group consisting of SEQ ID NOs: 8282-8314.

Embodiment 8e is an artificial ribonucleic acid (RNA) molecule comprising a poly(A) tail consisting of a nucleic acid sequence selected from the group consisting of SEQ ID NOs: 8282-8314.

Embodiment 9 is the artificial RNA molecule of embodiment 8 or 8b, wherein the 3′UTR element comprises a nucleic acid sequence of SEQ ID NO: 8201, SEQ ID NO: 8204, SEQ ID NO: 8209, or SEQ ID NO: 8211.

Embodiment 9a is the artificial RNA molecule of embodiment 8a, wherein the 3′UTR element consists of a nucleic acid sequence of SEQ ID NO: 8201, SEQ ID NO: 8204, SEQ ID NO: 8209, or SEQ ID NO: 8211.

Embodiment 10 is the artificial RNA molecule of any one of embodiments 8, 8b, or 9, wherein the 5′UTR element comprises a nucleic acid sequence of SEQ ID NO: 8214, SEQ ID NO: 8230, SEQ ID NO: 8235, SEQ ID NO: 8236, or SEQ ID NO: 8243.

Embodiment 10a is the artificial RNA molecule of any one of embodiments 8a, 8c, or 9a, wherein the 5′UTR element consists of a nucleic acid sequence of SEQ ID NO: 8214, SEQ ID NO: 8230, SEQ ID NO: 8235, SEQ ID NO: 8236, or SEQ ID NO: 8243.

Embodiment 11 is the artificial RNA molecule of any one of claims 8, 8b, 9, or 10, comprising the 3′UTR element and the 5′UTR element, wherein:
(1) the 3′UTR element comprises SEQ ID NO: 8209 and the 5′UTR element comprises SEQ ID NO: 8236;
(2) the 3′UTR element comprises SEQ ID NO: 8201 and the 5′UTR element comprises SEQ ID NO: 8236;
(3) the 3′UTR element comprises SEQ ID NO: 8209 and the 5′UTR element comprises SEQ ID NO: 8214; or
(4) the 3′UTR element comprises SEQ ID NO: 8209 and the 5′UTR element comprises SEQ ID NO: 8243.

Embodiment 11a is the artificial RNA molecule of any one of claims 8a, 8c, 9a, or 10a, comprising the 3′UTR element and the 5′UTR element, wherein:
(1) the 3′UTR element consists of SEQ ID NO: 8209 and the 5′UTR element consists of SEQ ID NO: 8236;
(2) the 3′UTR element consists of SEQ ID NO: 8201 and the 5′UTR element consists of SEQ ID NO: 8236;
(3) the 3′UTR element consists of SEQ ID NO: 8209 and the 5′UTR element consists of SEQ ID NO: 8214;
(4) the 3′UTR element consists of SEQ ID NO: 8209 and the 5′UTR element consists of SEQ ID NO: 8243.

Embodiment 12 is the artificial RNA molecule of embodiment 8, 8d, 9, 10, or 11, wherein the poly(A) tail comprises the nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

Embodiment 12a is the artificial RNA molecule of embodiment 12, wherein the poly(A) tail comprises the nucleic acid sequence of SEQ ID NO: 8306 or SEQ ID NO: 8307.

Embodiment 12b is the artificial RNA molecule of any one of embodiments 8, 8e, 9a, 10a, or 11a, wherein the poly(A) tail consists of the nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

Embodiment 12c is the artificial RNA molecule of embodiment 12b, wherein the poly(A) tail consists of the nucleic acid sequence of SEQ ID NO: 8306 or SEQ ID NO: 8307.

Embodiment 12d is the artificial RNA molecule of any one of embodiments 8-11a, comprising the 3′UTR element, the 5′UTR element, and the poly(A) tail, wherein:
(1) the 3′UTR element comprises SEQ ID NO: 8209, the 5′UTR element comprises SEQ ID NO: 8236; and the poly(A) tail comprises SEQ ID NO: 8306 or SEQ ID NO: 8307;
(2) the 3′UTR element comprises SEQ ID NO: 8201, the 5′UTR element comprises SEQ ID NO: 8236, and the poly(A) tail comprises SEQ ID NO: 8306 or SEQ ID NO: 8307;
(3) the 3′UTR element comprises SEQ ID NO: 8209, the 5′UTR element comprises SEQ ID NO: 8214, and the poly(A) tail comprises SEQ ID NO: 8306 or SEQ ID NO: 8307; or
(4) the 3′UTR element comprises SEQ ID NO: 8209, the 5′UTR element comprises SEQ ID NO: 8243, and the poly(A) tail comprises SEQ ID NO: 8306 or SEQ ID NO: 8307.

Embodiment 13 is a system for modifying DNA comprising:
(a) an artificial ribonucleic acid (RNA) molecule for enhancing expression of a polypeptide comprising a reverse transcriptase (RT) domain and optionally an endonuclease domain, the artificial RNA molecule comprising:
   (1) a nucleotide sequence encoding the polypeptide;
   (2) at least one of:
      (i) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269 and 8281; and/or
      (ii) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or
   (3) a poly(A) tail for enhancing expression of the polypeptide, wherein the poly(A) tail comprises a nucleic acid sequence selected from the group consisting of SEQ ID NOs: 8282-8314; and
(b) a template RNA (or DNA encoding the template RNA) comprising (e.g., from 5′ to 3′):
   (i) optionally a sequence that binds a target site in the DNA (e.g., a second strand of a site in a target genome);
      (ii) a sequence that binds the polypeptide;
      (iii) a heterologous object sequence; and
      (iv) optionally a 3′ target homology domain;
         wherein the heterologous object sequence comprises an alteration relative to a corresponding original sequence (e.g., a wild-type sequence), wherein the alteration improves the speed, fidelity, or speed and fidelity of target-primed reverse transcription by the reverse transcriptase.

Embodiment 13a is a system for modifying DNA comprising:
(a) an artificial ribonucleic acid (RNA) molecule for enhancing expression of a polypeptide comprising a reverse transcriptase (RT) domain and optionally an endonuclease domain, the artificial RNA molecule comprising a nucleotide sequence encoding the polypeptide; and at least one of:
   (i) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269 and 8281; and/or
   (ii) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and
(b) a template RNA (or DNA encoding the template RNA) comprising (e.g., from 5′ to 3′):
   (i) optionally a sequence that binds a target site in the DNA (e.g., a second strand of a site in a target genome);
   (ii) a sequence that binds the polypeptide;
   (iii) a heterologous object sequence; and
   (iv) optionally a 3′ target homology domain;
      wherein the heterologous object sequence comprises an alteration relative to a corresponding original sequence (e.g., a wild-type sequence), wherein the alteration improves the speed, fidelity, or speed and fidelity of target-primed reverse transcription by the reverse transcriptase.

Embodiment 13b is a system for modifying DNA comprising:
(a) an artificial ribonucleic acid (RNA) molecule for enhancing expression of a polypeptide comprising a reverse transcriptase (RT) domain and optionally an endonuclease domain, the artificial RNA molecule comprising (i) a nucleotide sequence encoding the polypeptide; and (ii) a poly(A) tail comprising a nucleic acid sequence selected from the group consisting of SEQ ID NOs: 8201-8233; and
(b) a template RNA (or DNA encoding the template RNA) comprising (e.g., from 5′ to 3′):
   (i) optionally a sequence that binds a target site in the DNA (e.g., a second strand of a site in a target genome);
   (ii) a sequence that binds the polypeptide;
   (iii) a heterologous object sequence; and
   (iv) optionally a 3′ target homology domain;
      wherein the heterologous object sequence comprises an alteration relative to a corresponding original sequence (e.g., a wild-type sequence), wherein the alteration improves the speed, fidelity, or speed and fidelity of target-primed reverse transcription by the reverse transcriptase.

Embodiment 14 is a system for modifying DNA comprising:
(a) an artificial ribonucleic acid (RNA) molecule for enhancing expression of a polypeptide comprising a reverse transcriptase (RT) domain and optionally an endonuclease domain, the artificial nucleic acid molecule comprising:
   (1) a nucleotide sequence encoding the polypeptide;
   (2) at least one of:
      (i) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269, and 8281; and/or
      (ii) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or
   (3) a poly(A) tail for enhancing expression of the polypeptide, wherein the poly(A) tail comprises a nucleic acid sequence selected from the group consisting of SEQ ID NOs: 8282-8314; and
(b) a template RNA (or DNA encoding the template RNA) comprising (e.g., from 5′ to 3′):
   (i) optionally a sequence that binds a target site in the DNA (e.g., a second strand of a site in a target genome),
      (ii) a sequence that binds the polypeptide,
      (iii) a heterologous object sequence, and
      (iv) optionally a 3′ target homology domain,
         preferably, the heterologous object sequence has one or both of the following characteristics: i) does not comprise self-complementary sequences, e.g., that form hairpin structures, e.g., under stringent conditions, or if a self-complementary sequence is present, it has one, two, or all of the following characteristics:
(1) each self-complementary sequence is no more than 10, 9, 8, 7, 6, 5, 4, or 3 nucleotides in length,
(2) the self-complementary sequence forms a hairpin comprising arms of no longer than 10, 9, 8, 7, 6, 5, 4, or 3 nucleotides in length, or
(3) the self-complementary sequence comprises at least 1, 2, 3, 4, or 5 positions of non-complementarity (e.g., mismatches or bulges) with its partner sequence, and
(4) does not comprise a repetitive sequence (e.g., a single-, di-, or tri-nucleotide repetitive sequence) or if a repetitive sequence is present it is of no more than 12, 11, 10, 9, 8, 7, or 6 nucleotides in length.

Embodiment 14a is a system for modifying DNA comprising:
(a) an artificial ribonucleic acid (RNA) molecule for enhancing expression of a polypeptide comprising a reverse transcriptase (RT) domain and optionally an endonuclease domain, the artificial nucleic acid molecule comprising a nucleotide sequence encoding the polypeptide; and at least one of:
   (i) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269, and 8281; and/or
   (ii) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and
(b) a template RNA (or DNA encoding the template RNA) comprising (e.g., from 5′ to 3′):
   (i) optionally a sequence that binds a target site in the DNA (e.g., a second strand of a site in a target genome),
   (ii) a sequence that binds the polypeptide,
   (iii) a heterologous object sequence, and
   (iv) optionally a 3′ target homology domain,
      preferably, the heterologous object sequence has one or both of the following characteristics: i) does not comprise self-complementary sequences, e.g., that form hairpin structures, e.g., under stringent conditions, or if a self-complementary sequence is present, it has one, two, or all of the following characteristics:
(1) each self-complementary sequence is no more than 10, 9, 8, 7, 6, 5, 4, or 3 nucleotides in length,
(2) the self-complementary sequence forms a hairpin comprising arms of no longer than 10, 9, 8, 7, 6, 5, 4, or 3 nucleotides in length, or
(3) the self-complementary sequence comprises at least 1, 2, 3, 4, or 5 positions of non-complementarity (e.g., mismatches or bulges) with its partner sequence, and
(4) does not comprise a repetitive sequence (e.g., a single-, di-, or tri-nucleotide repetitive sequence) or if a repetitive sequence is present it is of no more than 12, 11, 10, 9, 8, 7, or 6 nucleotides in length.

Embodiment 14b is a system for modifying DNA comprising:
(a) an artificial ribonucleic acid (RNA) molecule for enhancing expression of a polypeptide comprising a reverse transcriptase (RT) domain and optionally an endonuclease domain, the artificial nucleic acid molecule comprising (i) a nucleotide sequence encoding the polypeptide; and (ii) a poly(A) tail comprising a nucleic acid sequence selected from the group consisting of SEQ ID NOs: 8201-8233; and
(b) a template RNA (or DNA encoding the template RNA) comprising (e.g., from 5′ to 3′):
   (i) optionally a sequence that binds a target site in the DNA (e.g., a second strand of a site in a target genome),
   (ii) a sequence that binds the polypeptide,
   (iii) a heterologous object sequence, and
   (iv) optionally a 3′ target homology domain,
      preferably, the heterologous object sequence has one or both of the following characteristics: i) does not comprise self-complementary sequences, e.g., that form hairpin structures, e.g., under stringent conditions, or if a self-complementary sequence is present, it has one, two, or all of the following characteristics:
(1) each self-complementary sequence is no more than 10, 9, 8, 7, 6, 5, 4, or 3 nucleotides in length,
(2) the self-complementary sequence forms a hairpin comprising arms of no longer than 10, 9, 8, 7, 6, 5, 4, or 3 nucleotides in length, or
(3) the self-complementary sequence comprises at least 1, 2, 3, 4, or 5 positions of non-complementarity (e.g., mismatches or bulges) with its partner sequence, and
(4) does not comprise a repetitive sequence (e.g., a single-, di-, or tri-nucleotide repetitive sequence) or if a repetitive sequence is present it is of no more than 12, 11, 10, 9, 8, 7, or 6 nucleotides in length.

Embodiment 15 is the system of embodiment 13, 13a, 14, or 14a, wherein the 3′UTR element comprises a nucleic acid sequence of SEQ ID NO: 8201, SEQ ID NO: 8204, SEQ ID NO: 8209, SEQ ID NO: 8211, SEQ ID NO: 8256, or SEQ ID NO: 8281.

Embodiment 15a is the system of any one of embodiments 13, 13a, 14, or 14a, wherein the 3′UTR element consists of a nucleic acid sequence of SEQ ID NO: 8201, SEQ ID NO: 8204, SEQ ID NO: 8209, SEQ ID NO: 8211, SEQ ID NO: 8256, or SEQ ID NO: 8281.

Embodiment 16 is the system of any one of embodiments 13, 13a, 14, 14a, or 15, wherein the 5′UTR element comprises a nucleic acid sequence of SEQ ID NO: 8214, SEQ ID NO: 8230, SEQ ID NO: 8235, SEQ ID NO: 8236, SEQ ID NO: 8243, SEQ ID NO: 8259, or SEQ ID NO: 8263.

Embodiment 16a is the system of any one of embodiments 13, 13a, 14, 14a, 15, or 15a, wherein the 5′UTR element consists of a nucleic acid sequence of SEQ ID NO: 8214, SEQ ID NO: 8230, SEQ ID NO: 8235, SEQ ID NO: 8236, SEQ ID NO: 8243, SEQ ID NO: 8259, or SEQ ID NO: 8263.

Embodiment 17 is the system of any one of embodiments 13, 13a, 14, 14a, and 15-16a, wherein the artificial RNA molecule comprises the nucleotide sequence encoding the polypeptide, the 3′UTR element, and the 5′UTR element, and:
(1) the 3′UTR element comprises SEQ ID NO: 8209 and the 5′UTR element comprises SEQ ID NO: 8236;
(2) the 3′UTR element comprises SEQ ID NO: 8201 and the 5′UTR element comprises SEQ ID NO: 8236;
(3) the 3′UTR element comprises SEQ ID NO: 8209 and the 5′UTR element comprises SEQ ID NO: 8214;
(4) the 3′UTR element comprises SEQ ID NO: 8209 and the 5′UTR element comprises SEQ ID NO: 8243;
(5) the 3′UTR element comprises SEQ ID NO: 8281 and the 5′UTR element comprises SEQ ID NO: 8259; or
(6) the 3′UTR element comprises SEQ ID NO: 8281 and the 5′UTR element comprises SEQ ID NO: 8263.

Embodiment 17a is the system of any one of embodiments 13, 13a, 14, 14a, and 15-16a, wherein the artificial RNA molecule comprises the nucleotide sequence encoding the polypeptide, the 3′UTR, and the 5′UTR, and:
(1) the 3′UTR element consists of SEQ ID NO: 8209 and the 5′UTR element consists of SEQ ID NO: 8236;
(2) the 3′UTR element consists of SEQ ID NO: 8201 and the 5′UTR element consists of SEQ ID NO: 8236;
(3) the 3′UTR element consists of SEQ ID NO: 8209 and the 5′UTR element consists of SEQ ID NO: 8214;
(4) the 3′UTR element consists of SEQ ID NO: 8209 and the 5′UTR element consists of SEQ ID NO: 8243;
(5) the 3′UTR element consists of SEQ ID NO: 8281 and the 5′UTR element consists of SEQ ID NO: 8259; or
(6) the 3′UTR element consists of SEQ ID NO: 8281 and the 5′UTR element consists of SEQ ID NO: 8263.

Embodiment 17b is the system of embodiment 17, wherein the artificial RNA molecule comprises the nucleotide sequence encoding the polypeptide, the 3′UTR element, and the 5′UTR element, wherein the 3′UTR element comprises SEQ ID NO: 8201 and the 5′UTR element comprises SEQ ID NO: 8236.

Embodiment 17c is the system of embodiment 17, wherein the artificial RNA molecule comprises the nucleotide sequence encoding the polypeptide, the 3′UTR element, and the 5′UTR element, wherein the 3′UTR element comprises SEQ ID NO: 8209 and the 5′UTR element comprises SEQ ID NO: 8214.

Embodiment 18 is the system of any one of embodiments 13, 13b, 14, 14b, and 15-17c, wherein the poly(A) tail comprises a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

Embodiment 18a is the system of any one of embodiments 13, 13b, 14, 14b, and 15-18, wherein the poly(A) tail consists of a nucleic acid sequence of SEQ ID NO: 8294, SEQ ID NO: 8299, SEQ ID NO: 8306, SEQ ID NO: 8307, SEQ ID NO: 8309, SEQ ID NO: 8311, SEQ ID NO: 8312, or SEQ ID NO: 8314.

Embodiment 18b is the system of embodiment 18, wherein the poly(A) tail comprises the nucleic acid sequence of SEQ ID NO: 8306 or SEQ ID NO: 8307.

Embodiment 18c is the system of embodiments 18a, wherein the poly(A) tail consists of the nucleic acid sequence of SEQ ID NO: 8306 or SEQ ID NO: 8307.

Embodiment 18d is the system of any one of embodiments 13-16a, comprising the 3′UTR element, the 5′UTR element, and the poly(A) tail, wherein:
(1) the 3′UTR element comprises SEQ ID NO: 8209, the 5′UTR element comprises SEQ ID NO: 8236, and the poly(A) tail comprises SEQ ID NO: 8306 or SEQ ID NO: 8307;
(2) the 3′UTR element comprises SEQ ID NO: 8201, the 5′UTR element comprises SEQ ID NO: 8236, and the poly(A) tail comprises SEQ ID NO: 8306 or SEQ ID NO: 8307;
(3) the 3′UTR element comprises SEQ ID NO: 8209, the 5′UTR element comprises SEQ ID NO: 8214, and the poly(A) tail comprises SEQ ID NO: 8306 or SEQ ID NO: 8307;
(4) the 3′UTR element comprises SEQ ID NO: 8209, the 5′UTR element comprises SEQ ID NO: 8243, and the poly(A) tail comprises SEQ ID NO: 8306 or SEQ ID NO: 8307;
(5) the 3′UTR element comprises SEQ ID NO: 8281, the 5′UTR element comprises SEQ ID NO: 8259, and the poly(A) tail comprises SEQ ID NO: 8306 or SEQ ID NO: 8307; or
(6) the 3′UTR element comprises SEQ ID NO: 8281, the 5′UTR element comprises SEQ ID NO: 8263, and the poly(A) tail comprises SEQ ID NO: 8306 or SEQ ID NO: 8307.

Embodiment 19 is the system of any one of embodiments 13-18c, wherein the polypeptide comprises the reverse transcriptase domain and the endonuclease domain.

Embodiment 19a is the system of any one of embodiments 13-18c, wherein the polypeptide is a heterologous gene modifying polypeptide or a retrotransposon gene modifying polypeptide.

Embodiment 20 is the system of embodiment 19, wherein the endonuclease domain is a nickase domain.

Embodiment 20a is the system of embodiment 20, wherein the nickase domain is a Cas9 domain.

Embodiment 20b is the system of embodiment 20a, wherein the Cas9 domain is selected from a SpCas9 domain, a BlatCas9 domain, a Nme2 Cas9 domain, a PnpCas9 domain, a SauCas9 domain, a SauCas9-KKH domain, a SauriCas9 domain, a SauriCas9-KKH domain, a ScaCas9-Sc++ domain, a SpyCas9 domain, a SpyCas9-NG domain, a SpyCas9-SpRY domain, or a St1Cas9 domain.

Embodiment 20c is the system of any one of embodiments 20-20b, wherein the Cas9 domain comprises an N670A mutation, an N611A mutation, an N605A mutation, an N580A mutation, an N588A mutation, an N872A mutation, an N863A mutation, an N622A mutation, or an H840A mutation.

Embodiment 20d is the system of any one of embodiments 20-20c, wherein the reverse transcriptase domain is selected from a retrovirus reverse transcriptase domain.

Embodiment 20e is the system of embodiment 20d, wherein the retrovirus reverse transcriptase domain is a gamma retrovirus-derived reverse transcriptase domain.

Embodiment 20f is the system of embodiment 20e, wherein the gamma retrovirus-derived reverse transcriptase domain comprises the amino acid sequence of a reverse-transcriptase domain sequence from a family selected from AVIRE, BAEVM, FFV, FLV, FOAMV, GALV, KORV, MLVAV, MLVBM, MLVCB, MLVFF, MLVMS, PERV, SFV1, SFV3L, WMSV, and XMRV6.

Embodiment 20g is the system of embodiment 20e or 20f, wherein the gamma retrovirus-derived reverse transcriptase domain is not derived from PERV.

Embodiment 20 h is the system of any one of embodiments 20c-20f, wherein the reverse transcriptase domain comprises one, two, three, four, five, six, or more mutations corresponding to the following mutations D200N, L603W, T330P, D524G, E562Q, D583N, P51L, S67R, T197A, H204R, E302K, F309N, W313F, L435G, N454K, H594Q, L671P, E69K, or D653N in the reverse transcriptase domain of murine leukemia virus reverse transcriptase.

Embodiment 20i is the system of any one of embodiments 20-20h, wherein the polypeptide comprises the amino acid sequence of SEQ ID NO: 8257 or SEQ ID NO: 8371.

Embodiment 21 is the system of any one of embodiments 13-20i, wherein the template RNA further comprises an RT terminator sequence situated between the heterologous object sequence and either (i) or (ii).

Embodiment 22 is the system of any one of embodiments 13-21, wherein the heterologous object sequence encodes a target polypeptide or portion thereof or comprises a sequence that is the reverse complement of a sequence encoding the target polypeptide or portion thereof.

Embodiment 22a is the system of any one of embodiments 13-22, further comprising a second polypeptide or nucleic acid encoding a second polypeptide comprising an endonuclease domain.

Embodiment 22b is the system of any one of embodiments 13-22a, wherein (a) further comprises a DNA binding domain.

Embodiment 22c is the system of any one of embodiments 13-22b, wherein the second polypeptide further comprises a DNA binding domain.

Embodiment 23 is the system of any one of embodiments 13-22c, wherein the polypeptide comprises the reverse transcriptase domain and the endonuclease domain, and the endonuclease domain is a Cas9 domain, and the template RNA comprises:
(i) a gRNA spacer that is complementary to a first portion of a target gene, and optionally comprises one or more consecutive nucleotides starting with the 3′ end of the flanking nucleotides of the gRNA spacer;
(ii) a gRNA scaffold that binds to the Cas9 domain;
(iii) a heterologous object sequence comprising a mutation region to introduce a mutation into (e.g., to correct a mutation in) a second portion of the target gene (wherein optionally the heterologous sequence comprises, from 5′ to 3′ a post-edit homology region, a mutation region, and a pre-edit homology region), and
(iv) a primer binding site (PBS) sequence comprising at least 5, 6, 7, or 8 bases with 100% identity to a third portion of the target gene.

Embodiment 24 is the system of embodiment 23, wherein the target gene is a human PAH gene, and the template RNA comprises:
(i) a gRNA spacer that is complementary to a first portion of the human PAH gene, wherein the gRNA spacer has a sequence comprising the core nucleotides of a gRNA spacer sequence, preferably of Table 1A, Table 1B, Table 1C, or Table 1D in WO2023039435, which is herein incorporated by reference in its entirety, and optionally comprises one or more consecutive nucleotides starting with the 3′ end of the flanking nucleotides of the gRNA spacer, or wherein the gRNA spacer has a sequence of a spacer chosen from Tables 5A-5F, 8A-8D, E3, E3A, BB, E5, E5A, E6, or E6A in WO2023039435, which is herein incorporated by reference in its entirety;
(ii) a gRNA scaffold that binds to the Cas9 domain;
(iii) a heterologous object sequence comprising a mutation region to introduce a mutation into (e.g., to correct a mutation in) a second portion of the human PAH gene (wherein optionally the heterologous object sequence comprises, from 5′ to 3′, a post-edit homology region, a mutation region, and a pre-edit homology region); and
(iv) a primer binding site (PBS) sequence comprising at least 5, 6, 7, or 8 bases with 100% identity to a third portion of the human PAH gene.

Embodiment 25 is the system of any one of embodiments 13-24, wherein the template RNA comprises SEQ ID NO: 8258 (RNACS7570); SEQ ID NO: 8320 (RNACS229); SEQ ID NO: 8321 (RNACS1515); or SEQ ID NO: 8372.

Embodiment 26 is the system of any of embodiments 13-25, wherein the reverse transcriptase domain and the endonuclease domain are linked by a peptide linker.

Embodiment 27 is the system of any one of embodiments 13-26, wherein the target site is in a human genome.

Embodiment 28 is a reaction mixture comprising: a cell and the system of any one of embodiments 13-27.

Embodiment 28a is the reaction mixture of embodiment 28, wherein the cell is a T cell (e.g., a primary T cell).

Embodiment 29 is a reaction mixture comprising: a DNA comprising a target site and the system of any one of embodiments 13-27.

Embodiment 30 is the artificial RNA molecule of any one of embodiments 1-12c, wherein the artificial RNA molecule comprises one or more chemically modified nucleotides.

Embodiment 31 is a deoxyribonucleic acid (DNA) molecule encoding the artificial RNA molecule of any one of embodiments 1-12c.

Embodiment 32 is a pharmaceutical composition, comprising the artificial RNA molecule of any one of embodiments 1-12c and 30, the system of any one of embodiments 13-27, or one or more nucleic acids encoding the same, and a pharmaceutically acceptable excipient or carrier.

Embodiment 33 is the pharmaceutical composition of embodiment 32, wherein the pharmaceutically acceptable excipient or carrier is selected from the group consisting of a plasmid vector, a viral vector, a vesicle, and a lipid nanoparticle.

Embodiment 34 is the pharmaceutical composition of embodiment 33, wherein the viral vector is an adeno-associated virus.

Embodiment 35 is a host cell comprising the artificial RNA molecule or the system or the DNA molecule of any one of the preceding embodiments.

Embodiment 35a is the host cell of embodiment 35, wherein the host cell is a T cell (e.g., a primary T cell).

Embodiment 35b is the host cell of embodiment 35 or 35a, wherein the host cell is a mammalian cell.

Embodiment 35c is the host cell of embodiment 35b, wherein the mammalian cell is a human cell.

Embodiment 36 is a method of making the artificial RNA molecule of any one of embodiments 1-12c, the method comprising synthesizing the template RNA by in vitro transcription (e.g., solid state synthesis) or by introducing a DNA encoding the artificial RNA molecule into a host cell under conditions that allow for the production of the template RNA.

Embodiment 37 is a kit comprising:
(a) the system of any one of embodiments 13-27, the reaction mixture of embodiment 28 or 29, the DNA molecule of embodiment 31, or the pharmaceutical composition of any one of embodiments 32-34; and
(b) instructions for using the system, the reaction mixture, the DNA molecule, or the pharmaceutical composition.

Embodiment 38 is a lipid nanoparticle (LNP) comprising the artificial RNA molecule of any one of embodiments 1-12c and 30 or the system of any one of embodiments 13-27.

Embodiment 39 is a method for modifying a target site in genomic DNA in a cell, the method comprising: contacting the cell with the system of any one of embodiments 13-27 or one or more RNAs encoding the system, thereby modifying the target site in the genomic DNA in a cell.

Embodiment 40 is a method for treating a subject having a disease or condition associated with a genetic defect, the method comprising: administering to the subject the system of any one of embodiments 13-27, thereby treating the subject having a disease or condition associated with a genetic defect.

### EXAMPLES

The following examples of the invention are to further illustrate the nature of the invention. It should be understood that the following examples do not limit the invention and that the scope of the invention is to be determined by the appended claims.

### Example 1: Evaluating Expression and Activity of Exemplary Gene Modifying Polypeptide from mRNAs with Reference 5′ UTRs in U2OS Cells and Primary Mouse Hepatocytes

This example describes the use of exemplary gene modifying systems containing a template RNA and an mRNA encoding a gene modifying polypeptide to quantify the activity of mRNAs with various reference 5′ UTRs for targeted gene modifying function in a U2OS cell line and expression of the gene modifying polypeptide in the U2OS cell line and primary mouse hepatocytes.

In this example, an mRNA contained the following segments:
(1) a 5′ cap;
(2) one of the 5′ UTRs listed in Table 1;
(3) a coding sequence (CDS) encoding a gene modifying polypeptide and, in some embodiments, a HiBiT protein tag having a nucleotide sequence of SEQ ID NO: 8255 was added to the carboxy-terminal end of the gene modifying polypeptide;
(4) a 3′ UTR having a nucleotide sequence of SEQ ID NO: 8256; and
(5) a poly A tail comprising 80 adenine (A) residues.

In this example, the gene modifying polypeptide encoded by the mRNA contained:
(1) an endonuclease and/or DNA binding domain;
(2) a peptide linker; and
(3) a reverse transcriptase (RT) domain.

For example, a gene modifying polypeptide was encoded by mRNA RNAV209 and comprised the amino acid sequence of SEQ ID NO: 8257.

In this example, the template RNA co-delivered with the mRNA is RNACS7570 (SEQ ID NO: 8258), comprising the following nucleotide sequence: mG*mC*mC*rGrArArGrCrArCrUrGrCrArCrGrCrCrGrUrGrUrUrUrUrArGrAmGmCmUm AmGmAmAmAmUmAmGmCrArArGrUrUrArArArArUrArArGrGrCrUrArGrUrCrCrGrUr UrArUrCrAmAmCmUmUmGmAmAmAmAmAmGmUmGmGmCmAmCmCmGmAmGm UmCmGmGmUmGmCrArCrCrCrUrGrArCrGrUrArCrGrGrCrGrUrGrCrArGrUrG*mC*mU*mU. Nucleotide modifications are noted as follows: phosphorothioate linkages denoted by an asterisk (*), 2′-O-methyl groups denoted by an (m) preceding a nucleotide, ribonucleotide denoted by an ‘r’ preceding the nucleotide.

TABLE 1
Nucleotide sequences of 5′ UTRs used in this example

| 5′ UTR | | |
|---|---|---|
| Identifier | Nucleotide Sequence | SEQ ID NO |
| Reference 1 | AGGAAAUAAGAGAGAAAAGAAGAGUAAGAAGAAAUAUAAG | 8259 |
| | AGCCACC | |
| Reference 2 | AGGGAAAUAAGAGAGAAAAGAAGAGUAAGAAGAAAUAUAA | 8260 |
| | GAGCCACC | |
| Reference 3 | AGGGAAAUAAGAGAGAAAAGAAGAGUAAGAAGAAAUAUAA | 8261 |
| | GACCCCGGCGCCGCCACC | |
| Reference 4 | AGAGAAUAAACUAGUAUUCUUCUGGUCCCCACAGACUCAGA | 8262 |
| | GAGAACCCGCCACC | |
| Reference 5 | AGGCAAAAAUCAAAAUCAAUCAUCAUCACAACAUCAACAAU | 8263 |
| | CAAUCAUCAACACAUCAUCAAGACACCACC | |

To compare the efficiency of mRNAs with different reference 5′ UTRs for inducing targeted gene modification, a landing pad cell line called U2OS-BFP was used. U2OS-BFP cells were engineered to stably express Blue Fluorescence Protein (BFP). The template RNA was designed to convert the DNA sequence in the genome encoding BFP into that encoding GFP. The percentage of GFP positive cells (GFP %) among the harvested U2OS-BFP cell sample was used to assess the gene modifying activities of the system. In each reaction, the template RNA and the gene modifying polypeptide were invariant as the 5′ UTRs of the mRNA were varied.

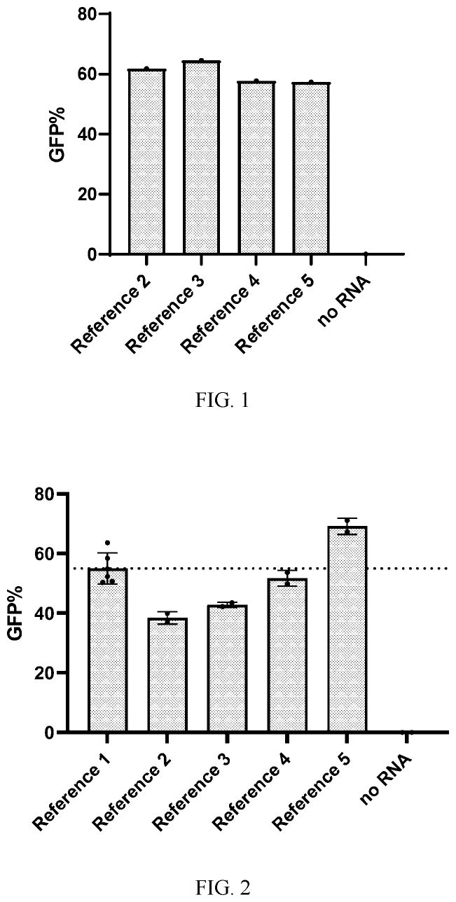

0.2 μg of the mRNA encoding the gene modifying polypeptide and 2 μg of template RNA were co-delivered on day 0 by nucleofection using Lonza's Amaxa™ Nucleofector 96-well Shuttle™ (Lonza; Basel, Switzerland) to 0.25 million U2OS-BFP cells. On day 4, the cells were harvested and examined by flow cytometry for BFP and GFP expression (FIG. 1). FlowJo was used to analyze the data and to determine GFP %. GraphPad Prism was used to examine correlation.

Using the mRNAs encoding non-HiBiT tagged gene modifying polypeptides, the GFP % induced by the reference 5′ UTRs ranged from 69.0% to 57.3%, indicating all the reference 5′ UTRs facilitated expression of the gene modifying polypeptide in a manner sufficient to facilitate the BFP to GFP editing.

The GFP % induced by gene modifying systems comprising mRNAs encoding HiBiT-tagged gene modifying polypeptides with reference 5′ UTRs ranged from 58.7% to 34.8%, indicating the reference 5′ UTRs also facilitated expression of the gene modifying polypeptide in a manner sufficient to facilitate BFP to GFP editing (FIG. 2).

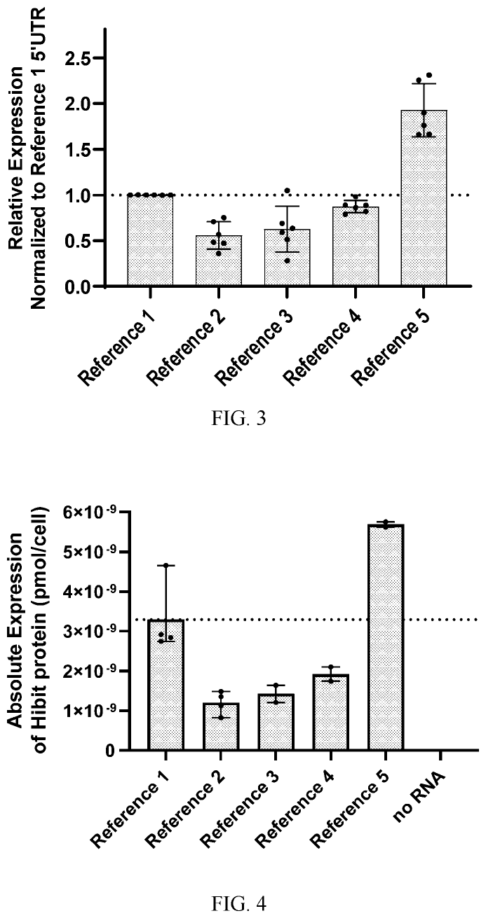

To directly compare the expression level of the gene modifying polypeptide from mRNAs with reference 5′ UTRs, 2 μg mRNA in the HiBiT set and 2 μg template RNA were co-delivered by nucleofection using Lonza's Amaxa™ Nucleofector 96-well Shuttle™ to 0.25 million U2OS cells that did not express BFP (U2OS-naïve). At 6 hours post-nucleofection, the cells were lysed and analyzed for HiBiT expression using Promega's Nano-Glo® HiBiT Lytic Detection System. The results show that gene modifying polypeptide expression levels varied approximately 2-fold above and below the normalization reference 5′UTR (Reference 1) across the reference 5′UTRs tested (FIG. 3).

To evaluate the effects of the reference 5′ UTRs on expression of the exemplary gene modifying polypeptide further in primary cells, primary mouse hepatocytes from two sources were used: Lonza's cryopreserved mouse hepatocytes (catalog number MCCP01) and hepatocytes freshly harvested from wild-type C57BL/6 mice. In each reaction, the template RNA and all other segments of mRNA besides the 5′ UTR remained identical. 2 μg mRNA encoding HiBiT-tagged gene modifying polypeptide and 4 μg template RNA were co-delivered to 0.1 million primary mouse hepatocytes by nucleofection using Lonza's Amaxa™ Nucleofector 96-well Shuttle™. At 24 hours post-nucleofection, the hepatocytes were lysed and analyzed for HiBiT expression using Promega's Nano-Glo® HiBiT Lytic Detection System.

The results show that all reference 5′UTRs successfully facilitated expression of the exemplary gene modifying polypeptide in the cryopreserved mouse hepatocytes (FIG. 4). Two reference 5′ UTRs, Reference 5 and Reference 1, induced higher gene modifying polypeptide expression than other reference 5′ UTRs in cryopreserved primary mouse hepatocytes.

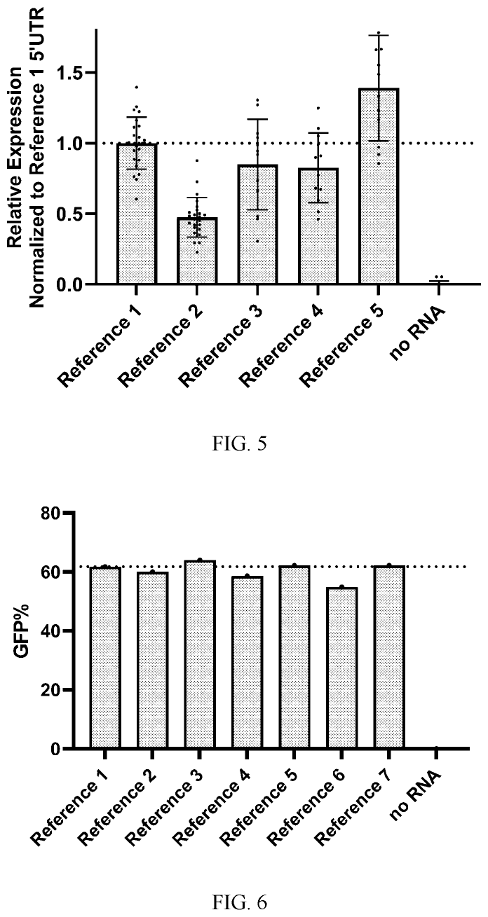

The expression levels of the HiBiT-tagged gene modifying polypeptide from mRNAs was also evaluated in hepatocytes freshly harvested from wild-type C57BL/6 mice at 24 hours post-nucleofection (FIG. 5). The results show that all reference 5′UTRs successfully facilitated expression of the exemplary gene modifying polypeptide in the freshly harvested primary mouse hepatocytes. Two reference 5′ UTRs, Reference 5 and Reference 1, induced higher gene modifying polypeptide expression than other reference 5′ UTRs in freshly harvested primary mouse hepatocytes.

Taken together, these results demonstrate that the tested reference 5′UTRs are capable of inducing gene modifying polypeptide expression in several different cell types, including different sources of primary hepatocytes, and that some reference 5′UTRs induce higher expression levels than others tested.

### Example 2: Evaluating Expression and Activity of Exemplary Gene Modifying Polypeptide from mRNAs with 5′ UTRs in U2OS Cells

This example describes the use of exemplary gene modifying systems containing a template RNA and an mRNA encoding a gene modifying polypeptide to quantify the activity of mRNAs with various 5′ UTRs for targeted gene modifying function and expression of gene modifying polypeptide in a U2OS cell line.

In this example, an mRNA contained the following segments:
(1) a 5′ cap;
(2) one of the 5′ UTRs listed in Table 2 or 5′UTR Reference 1;
(3) a coding sequence (CDS) encoding a gene modifying polypeptide and, in some embodiments, a HiBiT protein tag whose coding nucleotide sequence is SEQ ID NO: 8255 was added to the carboxy-terminal end of the gene modifying polypeptide;
(4) a 3′ UTR having a nucleotide sequence of SEQ ID NO: 8256; and
(5) a polyA tail comprising 80 A residues.

In this example, the gene modifying polypeptide encoded by the mRNA contained:
(1) an endonuclease and/or DNA binding domain;
(2) a peptide linker; and
(3) a reverse transcriptase (RT) domain.

The gene modifying polypeptide was encoded by mRNA RNAV209 (SEQ ID NO: 8257) and comprised the amino acid sequence provided in Example 1. In this example, the template RNA co-delivered with the mRNA is RNACS7570 (SEQ ID NO: 8258), comprising the nucleotide sequence given in Example 1. 5′UTRs tested in this Example were compared to Reference 1 5′UTR from Example 1.

TABLE 2
Nucleotide sequences of 5′ UTRs used in this Example

| 5′ UTR | | SEQ ID |
|---|---|---|
| Identifier | Nucleotide Sequence | NO |
| 50-1a | AGGAAAAUAACAUAAGACACGCAACUCAAACAUACUCAAACAACGCCACC | 8212 |
| 50-1b | AGGAUAAUAACAUAAGAUACUAACUAAUAAAUACUAAACUCAACGCCAC | 8213 |
| | C | |
| 50-1c | AGGAUAAUAUACUUACAUACUUACUAAUUAAUACUAAACUCAACGCCAC | 8214 |
| | C | |
| 50-2a | AGGAUCAACACAACAUACUAAAAAACUCUAUACAAUACAAAUUAGCCAC | 8215 |
| | C | |
| 50-2b | AGGAUCACACAACAACUCACACUACUCAUACAUCGACACACAUAGCCACC | 8216 |
| 50-2c | AGGAACAACACAACAAACUAAAAAACACAAUACAAAACAAAUAAGCCAC | 8217 |
| | C | |
| 50-2d | AGGAUCACACAACAACUCACACUACUCAUACAUCGACACACAUACCCCGG | 8218 |
| | CGCCGCCACC | |
| 50-3a | AGGACAAACAUUACACAACUAAAUAAACAACUAUCAAUUAACCGGCCACC | 8219 |
| 50-3b | AGGACAAACACAACACAACUAAAUAAACAACUAACAAUACAACCGCCACC | 8220 |
| 50-3c | AGGACAAUCACAAUCAUACUAAUAUAUCAACUAUCAAUUAAUCGGCCAC | 8221 |
| | C | |
| 50-4a | AGGACAUAUAAAACAACACUAUCACUAACAACUAUCAAAAUCAGGCCACC | 8222 |
| 50-4b | AGGACAUAUAUAACAACACUAUCACUAUCAACUAUCAUAAUCUCGCCACC | 8223 |
| 50-4c | AGGACACACAAAACAACACACAAAUCAACAACUAACAAAAUCACGCCACC | 8224 |
| 50-4d | AGGACACACAAAACAACACACAAAUCAACAACUAACAAAAUCACCCCCGG | 8225 |
| | CGCCGCCACC | |
| 50-5a | AGGAACAUAAACAUCAAAACUAAACAACAAAUCAACACAACAACGCCACC | 8226 |
| 50-5b | AGGAACAUAUACAUCAAUACUAAACAACUAAUCAACACAACUACGCCACC | 8227 |
| 50-5c | AGGAUCAUAAUACUACAAUACUAUACAACUAUCAAUACUAUCACGCCACC | 8228 |
| R50-6 | AGGAAACACAACAACACAACUAACAACACAACACAACUAAACAAGCCACC | 8229 |
| R50-7 | AGGAAAUAUAAUAAAUCAUAAAUAAUCAUAAUAAAUAAUCAAUAGCCAC | 8230 |
| | C | |
| 70-1a | AGGUCUAAUAUAUCAAAAUAACAUCUAAAAACAACAUAUCUAAAUUCUA | 8231 |
| | AUAUACACGUCGUUCGCCACC | |
| 70-1b | AGGACUAAUAAAUCAAAAUAACAACUAAAAACAACAUAACUAAAUACUA | 8232 |
| | AUAAACACGUCGAUCGCCACC | |
| 70-1c | AGGACUAAUAAAUCAAAAUAACAACUAAAAACAACAUAACAAAAUACAA | 8233 |
| | AUAAACACAACACAAGCCACC | |
| 70-1d | AGGACUAAUAAAUCAAAAUAACAACUAAAAACAACAUAACAAAAUACAA | 8234 |
| | AUAAACACAACACAACCCCGGCGCCGCCACC | |
| 70-2a | AGGUCAACAAUUUCUUAUCAUUAAAACGCUUUAUCCUUUAUUACCCUUU | 8235 |
| | UAUUUCUCUUAAUCUGCCACC | |
| 70-2b | AGGACAACAAUAACUCUAAUAAACAAACGAAUUCUAUUAUAUCACCACA | 8236 |
| | UUAUAUCUCUAAUCUGCCACC | |
| 70-2c | AGGACAACAAUAACAUCACAUAACAAAUACAAAUCUAACAACACACAAA | 8237 |
| | UAUAACUCUAAAUCAGCCACC | |
| 70-3a | AGGAACAAUUACAAAUAACAAUUACAAAAUCAGCAAACACAUAAACUCA | 8238 |
| | AGACCAGAUUAUAAAGCCACC | |
| 70-3b | AGGAACAAUUACAUAUAACAAUUACUUAAUCAGCAAACAUCAUUACUCU | 8239 |
| | CGACCACAUAUUAAAGCCACC | |
| 70-3c | AGGAACAATCACACATAACAATTACACAATCACCAAACACATCAACCTACA | 8240 |
| | CACCAATTATAAAGCCACC | |
| 70-4a | AGGAUCAUCAAAUCAACAUAAAUAAAGAAAAAACAAAGUCUUACCCGAA | 8241 |
| | AUCAUAAUUAAACACGCCACC | |
| 70-4b | AGGAUCAUCAACAACAUAAUAAUCAAAUAACAACAAUUCACAUCUCACU | 8242 |
| | UUCAUAAUUAAACACGCCACC | |
| 70-4c | AGGAUCAUCAAACACAUAAUAAUCAAAUAACAACACUACUCACAAACAC | 8243 |
| | UUACAAAUCAAACACGCCACC | |
| 70-4d | AGGAUCAUCAAACACAUAAUAAUCAAAUAACAACACUACUCACAAACAC | 8244 |
| | UUACAAAUCAAACACCCCCGGCGCCGCCACC | |
| 70-5a | AGGACCAUUACACUAUACUAUUUACUAUAAUUUUCAUAUACUAACUAUU | 8245 |
| | UCAAUCUUAUCCCGCGCCACC | |
| 70-5b | AGGACCAUAACAACUUACACUAUACUAUAAUAAUCAAAUACUUACAAAC | 8246 |
| | UCAAUCUUAUCACACGCCACC | |
| 70-5c | AGGACAACAACAAACAACACUAAACUACAAUAAUCAAAUACUAAAACAC | 8247 |
| | ACAAUCAAAUCACACGCCACC | |

To compare the efficiency of mRNAs with different 5′ UTRs for inducing targeted gene modification, a landing pad cell line called U2OS-BFP was used. U2OS-BFP cells were engineered to stably express Blue Fluorescence Protein (BFP). The template RNA was designed to convert the DNA sequence in the genome encoding BFP into that encoding GFP. The percentage of GFP positive cells (GFP %) among the harvested U2OS-BFP cell sample was used to assess the gene modifying activities of the system. In each reaction, the template RNA and the gene modifying polypeptides were invariant as the 5′ UTRs of the mRNA were varied.

0.2 μg of the mRNAs and 2 μg template RNA RNACS7570 (SEQ ID NO: 8258) were co-delivered on day 0 by nucleofection using Lonza's Amaxa™ Nucleofector 96-well Shuttle™ to 0.25 million U2OS-BFP cells. On day 4, the cells were harvested and examined by flow cytometry for BFP and GFP expression. FlowJo was used to analyze the data and to determine GFP % cell. GraphPad Prism was used to calculate significance and correlation.

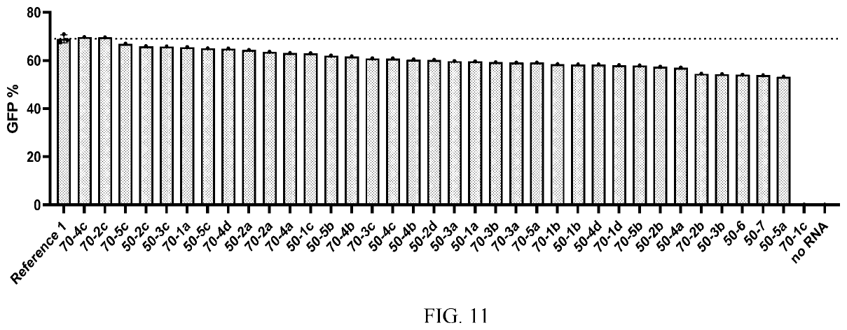

Using the mRNAs encoding non-HiBiT tagged gene modifying polypeptides, the GFP % ranged from 69.7% to 53.2%, indicating that many of the 5′ UTRs tested facilitated expression of the gene modifying polypeptide in a manner sufficient to facilitate BFP to GFP editing (FIG. 11). The levels of editing using gene modifying systems comprising mRNAs comprising several 5′UTRs (e.g., 70-4c (69.7%), 70-2c (69.6%), 70-5c (66.9%), 50-2c (65.9%), and 50-3c (65.8%)) were higher or comparable to the level obtained when using gene modifying systems comprising mRNA comprising reference 5′UTR Reference 1 in Example 1.

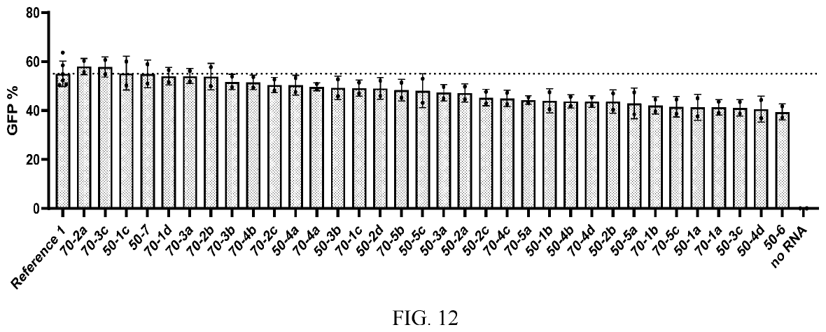

The % GFP positive cells obtained at day 4 after nucleofecting U2OS-BFP cells with mRNAs that encoded the HiBiT-tagged gene modifying polypeptides were also evaluated. The GFP % induced by HiBiT-tagged gene modifying systems comprising mRNAs with the 5′ UTRs ranged from 58.0% to 39.4%, indicating that many of the 5′ UTRs tested facilitated expression of the gene modifying polypeptide in a manner sufficient to facilitate BFP to GFP editing (FIG. 12). The levels of editing using gene modifying systems comprising mRNAs comprising several 5′UTRs (e.g., 70-2a (58.0%), 70-3c (57.7%), 50-1c (55.2%), 50-7 (54.9%), 70-1d (54.0%), 70-3a (54.0%), 70-2b (53.8%)) were higher or comparable to the level obtained when using gene modifying systems comprising mRNA comprising Reference 15′ UTR.

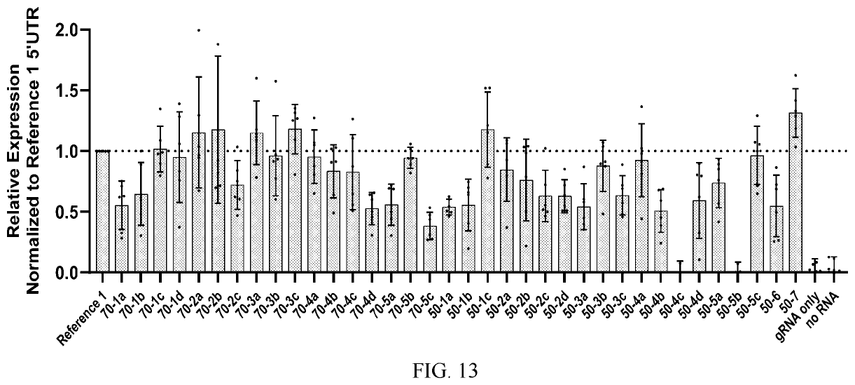

To directly compare the expression level of the gene modifying polypeptide from mRNAs with different 5′ UTRs, 2 μg mRNA encoding HiBiT-tagged gene modifying polypeptide and 2 μg template RNA were co-delivered to 0.25 million U2OS-naïve cells that did not express BFP by nucleofection using Lonza's Amaxa™ Nucleofector 96-well Shuttle™ At 6 hours post-nucleofection, the cells were lysed and analyzed for HiBiT expression using Promega's Nano-Glo® HiBiT Lytic Detection System. The results show that several 5′ UTRs, such as 50-7, 70-3c, 50-1c, 70-2a, 70-2b, and 70-3a, enabled 15%-30% higher expression of gene modifying polypeptide than the mRNAs comprising Reference 1 5′ UTR (FIG. 13).

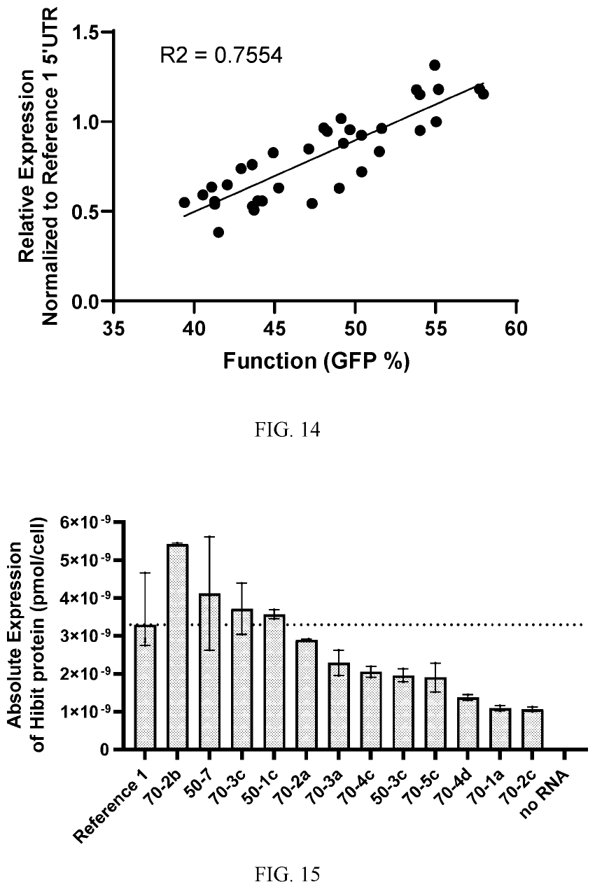

Gene modifying polypeptide expression was also evaluated in U2OS-naïve cells (do not express BFP) from mRNAs encoding HiBiT-tagged gene modifying polypeptide comprising the various 5′ UTRs (normalized to expression from mRNAs comprising Reference 1 5′UTR) against % GFP obtained from nucleofecting HiBiT-tagged gene modifying systems comprising the same mRNAs. Expression levels were obtained 6 hours post-nucleofection (FIG. 14). The data allowed analysis of the correlation between the expression level of the gene modifying polypeptide shown in FIG. 13 and the gene modifying activities shown in FIG. 12. Two-tailed Pearson correlation was used to calculate the correlation, resulting in an R2 of 0.7554. The results suggest that the expression level of the exemplary gene modifying polypeptide, as expressed from mRNAs comprising the 5′UTRs, is well-correlated with the gene modifying function. Accordingly, improvements in gene modifying function can likely be attributed to stronger expression of the gene modifying polypeptide from the mRNAs with these novel 5′ UTRs. The results suggest that the editing activity of gene modifying systems comprising mRNA encoding gene modifying polypeptides can be improved using 5′UTRs that increase gene modifying polypeptide expression as described herein.

### Example 3: Evaluating Expression of Exemplary Gene Modifying Polypeptide from mRNAs with 5′ UTRs in Primary Mouse Hepatocytes

This example describes the quantification of expression of an exemplary gene modifying polypeptide in exemplary gene modifying systems comprising mRNAs encoding the gene modifying polypeptide and various 5′ UTRs in primary mouse hepatocytes. In this example, an mRNA contained the following segments:
(1) a 5′ cap;
(2) one of the 5′ UTRs listed in Table 2 or 5′UTR Reference 1;
(3) a coding sequence (CDS) encoding a gene modifying polypeptide and a HiBiT protein tag whose coding sequence is SEQ ID NO: 8255 was added to the carboxy-terminal end of the gene modifying polypeptide;
(4) a 3′ UTR whose nucleotide sequence is of SEQ ID NO: 8256; and
(5) a polyA tail comprising 80 A residues.

In this example, the gene modifying polypeptide encoded by the mRNA contained:
(1) an endonuclease and/or DNA binding domain;
(2) a peptide linker; and
(3) a reverse transcriptase (RT) domain.

The gene modifying polypeptide was the same as that described in Examples 1-2.

In this example, the template RNA co-delivered with the mRNA is RNACS7570 (SEQ ID NO: 8258), comprising the nucleic acid sequence and modifications given in Example 1. 5′UTRs tested in this Example were compared to Reference 1 5′UTR from Example 1 (see Table 2).

To evaluate the effectiveness of 5′ UTRs to promote expression of an exemplary gene modifying polypeptide further in primary cells, primary mouse hepatocytes from two sources were used: Lonza's cryopreserved mouse hepatocytes (catalog number MCCP01) and hepatocytes freshly harvested from wild-type C57BL/6 mice. In each reaction, the template RNA and gene modifying polypeptides were invariant as the 5′ UTRs of the mRNA were varied.

2 μg mRNA encoding HiBiT-tagged gene modifying polypeptide and 4 μg template RNA were co-delivered to 0.1 million primary mouse hepatocytes by nucleofection using Lonza's Amaxa™ Nucleofector 96-well Shuttle™. At 24 hours post-nucleofection, the hepatocytes were lysed and analyzed for HiBiT expression using Promega's Nano-Glo® HiBiT Lytic Detection System. The Absolute Expression of HiBiT protein (pmol/cell) was calculated using a standard HiBiT quantity ladder prepared using the HiBiT reference protein from Promega (catalog #N3010) and the cell number ladder prepared with ThermoFisher's PrestoBlue™ Cell Viability Reagent (Catalog number: A13261). The results show that several 5′ UTRs, such as 70-2b, 50-7, 70-3c, and 50-1c, enabled higher or comparable expression of gene modifying polypeptide than the Reference 1 5′ UTR (FIG. 15).

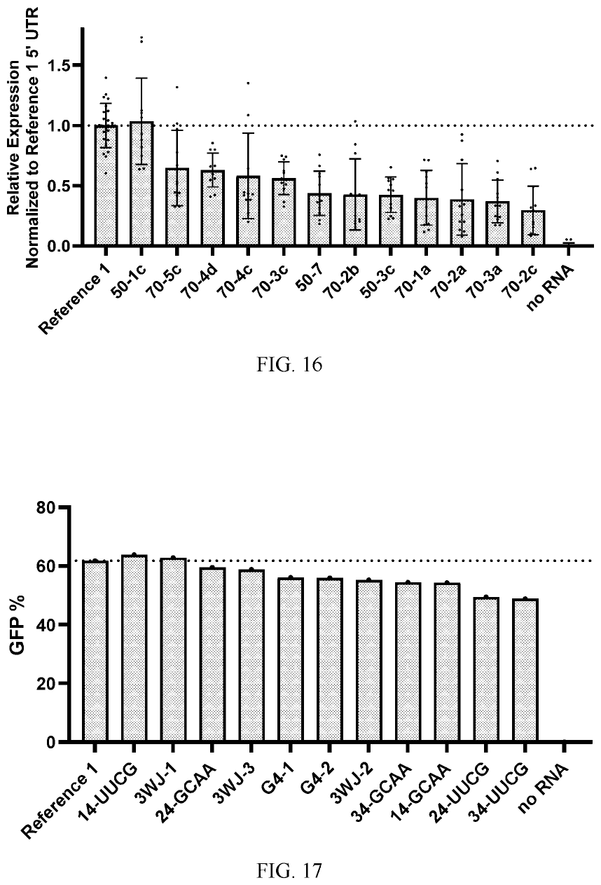

The expression of the HiBiT-tagged gene modifying polypeptide from mRNAs comprising the various 5′ UTRs was also evaluated at 24 hours post-nucleofection in hepatocytes freshly harvested from wild-type C57BL/6 mice and normalized to the expression level of gene modifying polypeptide from an mRNA comprising Reference 1 5′UTR. The results show that the 50-1c 5′ UTR enabled comparable expression of gene modifying polypeptide compared to the mRNA comprising Reference 1 5′ UTR (FIG. 16). Additionally, the tested 5′ UTRs are capable of enabling expression of an exemplary gene modifying polypeptide in primary hepatocytes from two different sources, and that some 5′ UTRs enabled comparable or higher expression than the Reference 1 5′UTR.

### Example 4: Evaluating Expression and Activity of Exemplary Gene Modifying Polypeptide from mRNAs with 3′ UTRs in U2OS Cells and Primary Mouse Hepatocytes

This example describes the use of exemplary gene modifying systems containing a template RNA and an mRNA encoding a gene modifying polypeptide to quantify the activity of mRNAs with various reference 3′ UTRs for targeted gene modifying function in a U2OS cell line and expression of gene modifying polypeptide in a U2OS cell line and primary mouse hepatocytes.

In this example, an mRNA contained the following segments:
(1) a 5′ cap;
(2) a 5′ UTR having a nucleotide sequence of SEQ ID NO: 8260;
(3) a coding sequence (CDS) encoding a gene modifying polypeptide and, in some embodiments, a HiBiT protein tag whose coding nucleotide sequence is SEQ ID NO: 8255 was added to the carboxy-terminal end of the gene modifying polypeptide;
(4) one of the 3′ UTRs listed in Table 3; and
(5) a poly A tail comprising 80 A residues.

In this example, the gene modifying polypeptide encoded by the mRNA contained:
(1) an endonuclease and/or DNA binding domain;
(2) a peptide linker; and
(3) a reverse transcriptase (RT) domain.

The 3′ UTRs listed in Table 3 include a terminal CUAG as a consequence of nucleic acid synthesis methodologies employed. The experiments described herein may be performed using 3′UTRs lacking the terminal CUAG and similar results are expected.

The gene modifying polypeptide was the same as that described in Examples 1-3.

In this example, the template RNA co-delivered with the mRNA was RNACS7570 (SEQ ID NO: 8258), described above in Example 1.

TABLE 3
Nucleotide sequences of UTRs used in this example

| 3′ UTR | | SEQ ID |
|---|---|---|
| Identifier | Nucleotide Sequence | NO |
| Reference 1 | GCUGGAGCCUCGGUGGCCAUGCUUCUUGCCCCUUGGGCCUCCCCCCAG | 8256 |
| | CCCCUCCUCCCCUUCCUGCACCCGUACCCCCGUGGUCUUUGAAUAAAG | |
| | UCUGACUAG | |
| Reference 2 | GCUGGAGCCUCGGUGGCCUAGCUUCUUGCCCCUUGGGCCUCCCCCCAG | 8264 |
| | CCCCUCCUCCCCUUCCUGCACCCGUACCCCCGUGGUCUUUGAAUAAA | |
| | GUCUGAGUGGGCGGCCUAG | |
| Reference 3 | UCCAUAAAGUAGGAAACACUACAGCUGGAGCCUCGGUGGCCAUGCUUC | 8265 |
| | UUGCCCCUUGGGCCUCCCCCCAGCCCCUCCUCCCCUUCCUGCACCCGUA | |
| | CCCCCCGCAUUAUUACUCACGGUACGAGUGGUCUUUGAAUAAAGUCUG | |
| | AGUGGGCGGCCUAG | |
| Reference 4 | CUCGAGCUGGUACUGCAUGCACGCAAUGCUAGCUGCCCCUUUCCCGUC | 8266 |
| | CUGGGUACCCCGAGUCUCCCCCGACCUCGGGUCCCAGGUAUGCUCCCA | |
| | CCUCCACCUGCCCCACUCACCACCUCUGCUAGUUCCAGACACCUCCCAA | |
| | GCACGCAGCAAUGCAGCUCAAAACGCUUAGCCUAGCCACACCCCCACG | |
| | GGAAACAGCAGUGAUUAACCUUUAGCAAUAAACGAAAGUUUAACUAA | |
| | GCUAUACUAACCCCAGGGUUGGUCAAUUUCGUGCCAGCCACACCCUGG | |
| | AGCUAGCCUAG | |
| Reference 5 | UUGUGUAUGCGUUAAUAAAAAGAAGGAACUCGUAAAAACUCAAUGUA | 8267 |
| | UUUCUGAGGAAGCGUGGUGCAUAAUGCCACGCAGCGUCUGCAUAACU | |
| | UUUAUUAUUUCUUUUAUUAAUCAACAAACUAG | |
| Reference 6 | UUGUGUAUGCGUUAAUAAAAAGAAGGAACUCGUACUAG | 8268 |
| Reference 7 | GCUGCCUUCUGCGGGGCUUGCCUUCUGGCCAUGCCCUUCUUCUCUCC | 8269 |
| | CUUGCACCUGUACCUCUUGGUCUUUGAAUAAAGCCUGAGUAGGAAGC | |
| | UAG | |

To compare the efficiency of mRNAs with different reference 3′ UTRs for inducing targeted gene modification, a landing pad cell line called U2OS-BFP was used. U2OS-BFP cells were engineered to stably express Blue Fluorescence Protein (BFP). The template RNA was designed to convert the DNA sequence in the genome encoding BFP into that encoding GFP. The percentage of GFP positive cells (GFP %) among the harvested U2OS-BFP cell sample was used to assess the gene modifying activities of the system. In each reaction, the template RNA and gene modifying polypeptides were invariant as the 3′ UTRs of the mRNA were varied.

0.2 μg of the mRNAs encoding the gene modifying polypeptide and 2 μg of template RNA RNACS7570 (SEQ ID NO: 8258) were co-delivered on day 0 by nucleofection using Lonza's Amaxa™ Nucleofector 96-well Shuttle™ to 0.25 million U2OS-BFP cells. On day 4, the cells were harvested and examined by flow cytometry for BFP and GFP expression. FlowJo was used to analyze the data and to determine GFP % cells. GraphPad Prism was used to examine correlation.

The results show the percentages of GFP positive U2OS-BFP cells (GFP %) detected by flow cytometry on day 4 (FIG. 6). Using the mRNAs encoding non-HiBiT tagged gene modifying polypeptide, the GFP % induced by mRNAs with the reference 3′ UTRs ranges from 69.0% to 54.8%, indicating all the reference 3′ UTRs facilitated expression of the gene modifying polypeptide in a manner sufficient to facilitate BFP to GFP editing.

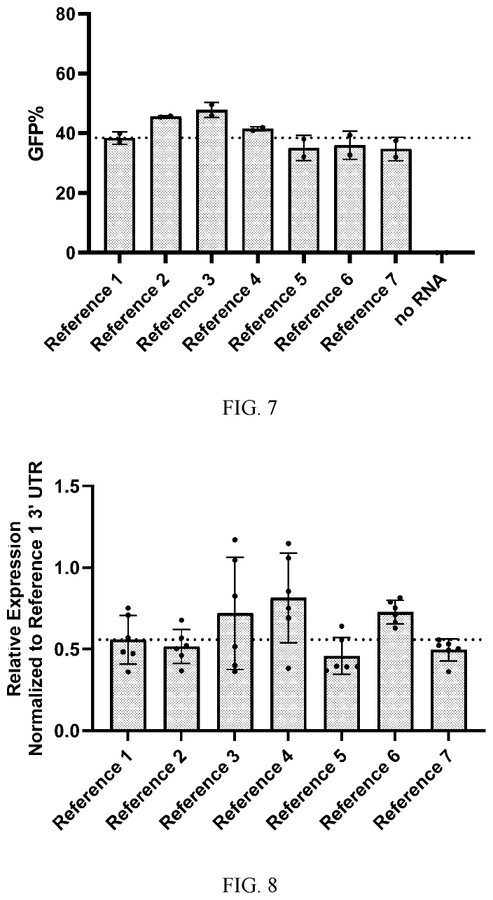

The % GFP positive cells were also examined in U2OS-BFP cells 4 days post-nucleofecting mRNAs that encoded the HiBiT-tagged gene modifying peptide using flow cytometry (FIG. 7). The GFP % induced by HiBiT-tagged gene modifying systems comprising mRNAs with reference 3′ UTRs ranged from 47.8% to 34.8%, indicating the reference 3′ UTRs also facilitated expression of the gene modifying polypeptide in a manner sufficient to facilitate BFP to GFP editing.

To directly compare the expression level of the gene modifying polypeptide from mRNAs with reference 3′ UTRs, 2 μg mRNA in the HiBiT set and 2 μg template RNA were co-delivered by nucleofection using Lonza's Amaxa™ Nucleofector 96-well Shuttle™ to 0.25 million U2OS cells that did not express BFP (U2OS-naïve). At 6 hours post-nucleofection, the cells were lysed and analyzed for HiBiT expression using Promega's Nano-Glo® HiBiT Lytic Detection System. The results show that gene modifying polypeptide expression levels varied approximately 0.5-fold or less above and below the normalization reference 3′UTR (Reference 1) across the reference 3′UTRs tested (FIG. 8).

To evaluate the effects of reference 3′ UTRs on expression of the exemplary gene modifying polypeptide further in primary cells, primary mouse hepatocytes from two sources were used: Lonza's cryopreserved mouse hepatocytes (catalog number MCCP01) and hepatocytes freshly harvested from wild-type C57BL/6 mice. In each reaction, the template RNA and all other segments of mRNA besides the 3′ UTR remained identical.

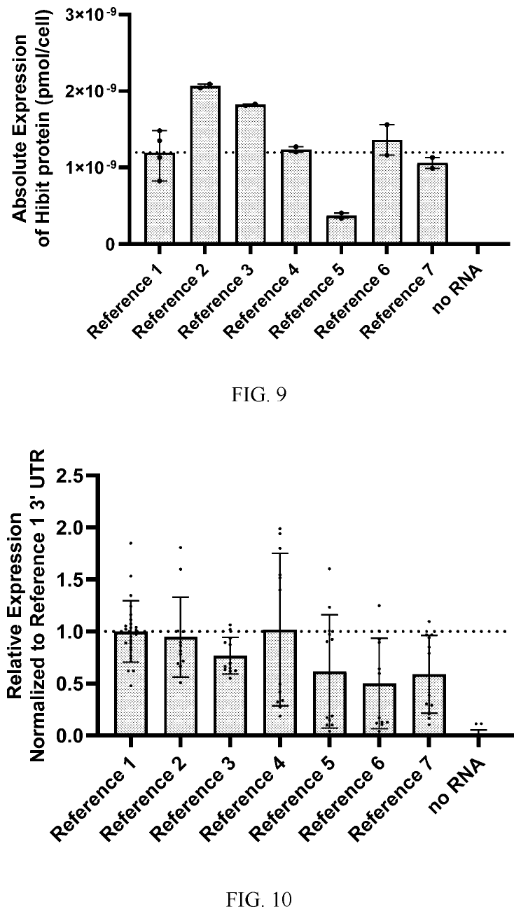

2 μg mRNA encoding HiBiT tagged gene modifying polypeptide and 4 μg template RNA were co-delivered to 0.1 million primary mouse hepatocytes by nucleofection using Lonza's Amaxa™ Nucleofector 96-well Shuttle™. At 24 hours post-nucleofection, the hepatocytes were lysed and analyzed for HiBiT expression using Promega's Nano-Glo® HiBiT Lytic Detection System. The results show that all reference 3′UTRs successfully facilitated expression of the exemplary gene modifying polypeptide in the cryopreserved mouse hepatocytes (FIG. 9). Two reference 3′ UTRs, Reference 2 and Reference 3, induced higher gene modifying polypeptide expression than other reference 3′ UTRs in cryopreserved primary mouse hepatocytes.

The expression levels of the HiBiT-tagged gene modifying polypeptide from RNAs was also evaluated in hepatocytes from freshly harvested wild-type C57BL/6 mice 24 hours post-nucleofection. The results show that all reference 3′UTRs successfully facilitated expression of the exemplary gene modifying polypeptide in the freshly harvested primary mouse hepatocytes (FIG. 10). Three reference 3′ UTRs, Reference 1, Reference 2, and Reference 4, induced higher gene modifying polypeptide expression than other reference 3′ UTRs in freshly harvested primary mouse hepatocytes.

Taken together, these results demonstrate that the tested reference 3′UTRs are capable of inducing gene modifying polypeptide expression in several different cell types, including different sources of primary hepatocytes, and that some reference 3′UTRs induce higher expression levels than others tested.

### Example 5: Evaluating Expression and Activity of Exemplary Gene Modifying Polypeptide from mRNAs with 3′ UTRs in U2OS Cells

This example describes the use of exemplary gene modifying systems containing a template RNA and an mRNA encoding a gene modifying polypeptide to quantify the activity of mRNAs with various 3′ UTRs for targeted gene modifying function and expression of gene modifying polypeptide in a U2OS cell line.

In this example, an mRNA contained the following segments:
(1) a 5′ cap;
(2) a 5′ UTR having a nucleotide sequence of SEQ ID NO: 8260;
(3) a coding sequence (CDS) encoding a gene modifying polypeptide and, in some embodiments, a HiBiT protein tag whose coding nucleotide sequence is SEQ ID NO: 8255 was added to the carboxy-terminal end of the gene modifying polypeptide;
(4) one of the 3′ UTRs listed in Table 3 or Table 4 and
(5) a poly A tail comprising 80 A residues.

In this example, the gene modifying polypeptide encoded by the mRNA contained:
(1) an endonuclease and/or DNA binding domain;
(2) a peptide linker; and
(3) a reverse transcriptase (RT) domain.

The gene modifying polypeptide was the same as that described in Examples 1-4.

In this example, the template RNA co-delivered with the mRNA was RNACS7570 (SEQ ID NO: 8258), described in Example 1. 3′UTRs tested in this Example were compared to Reference 1 3′UTR from Example 4.

TABLE 4
Nucleotide sequences of 3′ UTRs used in this example

| 3′ UTR | | SEQ |
|---|---|---|
| Identifier | Nucleotide Sequence | ID NO |
| 14-UUCG | UUGCGUUCGCGCAA | 8201 |
| 14-GCAA | ACGCUGCAAAGCGU | 8202 |
| 24-UUCG | AGACGCGAACUUCGGUUCGCGUCU | 8204 |
| 24-GCAA | GCUCCCGCUAGCAAUAGCGGGAGU | 8203 |
| 34-UUCG | CGGGACCUAACGUGAUUCGUCACGUUAGGUCCCG | 8205 |
| 34-GCAA | GCAACGACGACCCGAGCAAUCGGGUCGUCGUUGU | 8206 |
| 3WJ-1 | UUGCCUAGUGUUGAGGUGUUCGCACCUCAACUUCAUAGAUCCGCAAGG | 8207 |
| | AUCUAUCUAGGCAA | |
| 3WJ-2 | AUCGCUCGAGGUGAAGAGUUCGCUUUUCAUCAAAUCGAAGCCGCAAGG | 8208 |
| | UUUCGACGAGCGAU | |
| 3WJ-3 | UUGCCAUGUGUAUGUGGGUUUUUUUUUUCCCACAUACUCUGAUGAUCC | 8209 |
| | UUUUUUUUUUGGAUCAUUCAUGGCAA | |
| G4-1 | GGGGAGUGGGAGGGACAGAGGG | 8210 |
| G4-2 | GGGAACUGUGAUGGGGUGGGCAGGGCUGGGAGAACUUUGUACAG | 8211 |

To compare the efficiency of mRNAs with different 3′ UTRs for inducing targeted gene modification, a landing pad cell line called U2OS-BFP was used. U2OS-BFP cells were engineered to stably express Blue Fluorescence Protein (BFP). The template RNA was designed to convert the DNA sequence in the genome encoding BFP into that encoding GFP. The percentage of GFP positive cells (GFP %) among the harvested U2OS-BFP cell sample was used to assess the gene modifying activities of the system. In each reaction, the template RNA and gene modifying polypeptides were invariant as the 3′ UTRs of the mRNA were varied.

0.2 μg of the mRNAs and 2 μg template RNA were co-delivered on day 0 by nucleofection using Lonza's Amaxa™ Nucleofector 96-well Shuttle™ to 0.25 million U2OS-BFP cells. On day 4, the cells were harvested and examined by flow cytometry for BFP and GFP expression. FlowJo was used to analyze the data and to determine GFP % cells. GraphPad Prism was used to calculate significance and correlation.

Using the mRNAs encoding non-HiBiT tagged gene modifying polypeptides, the GFP % ranged from 63.9% to 48.8%, indicating that many of the 3′ UTRs tested facilitated expression of the gene modifying polypeptide in a manner sufficient to facilitate BFP to GFP editing (FIG. 17). The levels of editing using gene modifying systems comprising mRNAs comprising several 3′ UTRs (e.g., 14-UUCG (63.9%), 3WJ-1 (62.9%), 24-GCAA (59.5%), and 3WJ-3 (58.9%)) were higher or comparable to the level obtained when using gene modifying systems comprising mRNA comprising the Reference 1 3′ UTR in Example 4.

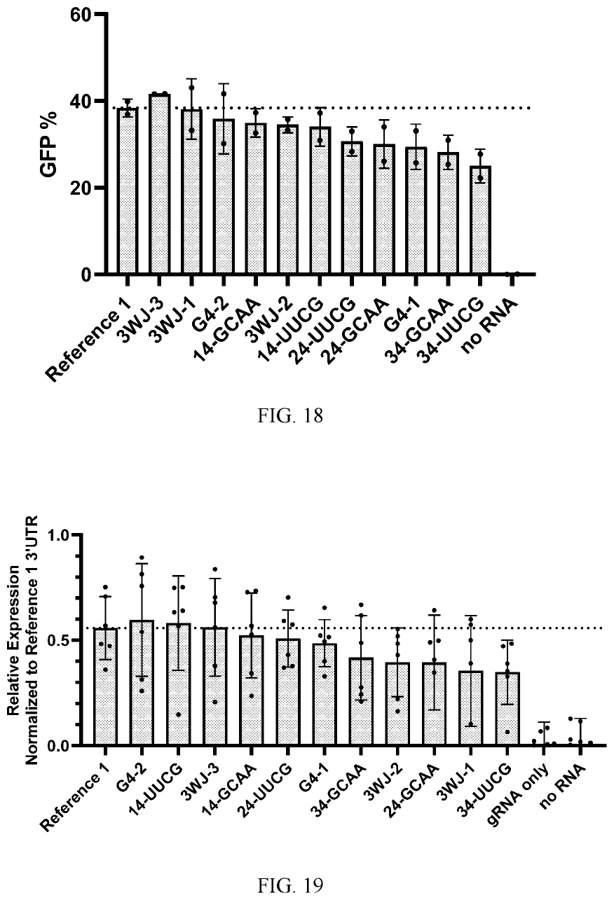

The % GFP positive cells obtained on day 4 after nucleofecting U2OS-BFP cells with mRNAs that encoded the HiBiT-tagged gene modifying peptide were also evaluated. The GFP % induced by the gene modifying systems comprising mRNAs encoding HiBiT tagged gene modifying polypeptide with the 3′ UTRs ranged from 41.6% to 25.0%, indicating that many of the 3′ UTRs tested facilitated expression of the gene modifying polypeptide in a manner sufficient to facilitate BFP to GFP editing (FIG. 18). The levels of editing using gene modifying systems comprising mRNAs comprising several 3′UTRs (e.g., 3WJ-3 (41.6%), 3WJ-1 (38.1%), G4-2 (35.9%), 14-GCAA (35.0%), 3WJ-2 (34.5%), and 14-UUCG (35.0%)) were higher or comparable to the level obtained when using gene modifying systems comprising mRNA comprising the Reference 1 3′ UTR.

To directly compare the expression level of the gene modifying polypeptide from mRNAs with different 3′ UTRs, 2 μg mRNA encoding HiBiT tagged gene modifying polypeptide and 2 μg template RNA were co-delivered to 0.25 million U2OS-naïve cells that did not express BFP by nucleofection using Lonza's Amaxa™ Nucleofector 96-well Shuttle™. At 6 hours post-nucleofection, the cells were lysed and analyzed for HiBiT expression using Promega's Nano-Glo® HiBiT Lytic Detection System. The results show that several 3′ UTRs, such as G4-2, 3UTR14-UUCG, 3WJ-3, 3UTR14-GCAA, 3UTR24-UUCG, and G4-1 enabled a higher or similar expression of gene modifying polypeptide comparing to the mRNA comprising the Reference 1 3′ UTR (FIG. 19). The dotted line marks the expression from the mRNA comprising the reference 3′ UTR Reference

### Example 6: Evaluating Expression of Exemplary Gene Modifying Polypeptide from mRNAs with 3′ UTRs in Primary Mouse Hepatocytes

This example describes the quantification of expression of an exemplary gene modifying polypeptide in exemplary gene modifying systems comprising mRNAs encoding the gene modifying polypeptide and various 3′ UTRs in primary mouse hepatocytes.

In this example, an mRNA contained the following segments:
(1) a 5′ cap;
(2) a 5′ UTR whose nucleotide sequence is of SEQ ID NO: 8260;
(3) a coding sequence (CDS) encoding a gene modifying polypeptide and a HiBiT protein tag whose coding nucleotide sequence is SEQ ID NO: 8255 was added to the carboxy-terminal end of the gene modifying polypeptide;
(4) one of the 3′ UTRs listed in Table 4 or 3′ UTR Reference 1; and
(5) a poly A tail comprising 80 A residues.

In this example, the gene modifying polypeptide encoded by the mRNA contained:
(1) an endonuclease and/or DNA binding domain;
(2) a peptide linker; and
(3) a reverse transcriptase (RT) domain.

The gene modifying polypeptide was the same as that provided in Example 1.

In this example, the template RNA co-delivered with the mRNA was RNACS7570 (SEQ ID NO: 8258), comprising the nucleic acid sequence and modifications given in Example 1. 3′UTRs tested in this Example were compared to Reference 1 3′UTR from Example 4.

To evaluate the effectiveness of 3′ UTRs to promote expression of an exemplary gene modifying polypeptide further in primary cells, primary mouse hepatocytes from two sources were used: Lonza's cryopreserved mouse hepatocytes (catalog number MCCP01) and hepatocytes freshly harvested from wild-type C57BL/6 mice. In each reaction, the template RNA and gene modifying polypeptides were invariant as the 3′ UTRs of the mRNA were varied.

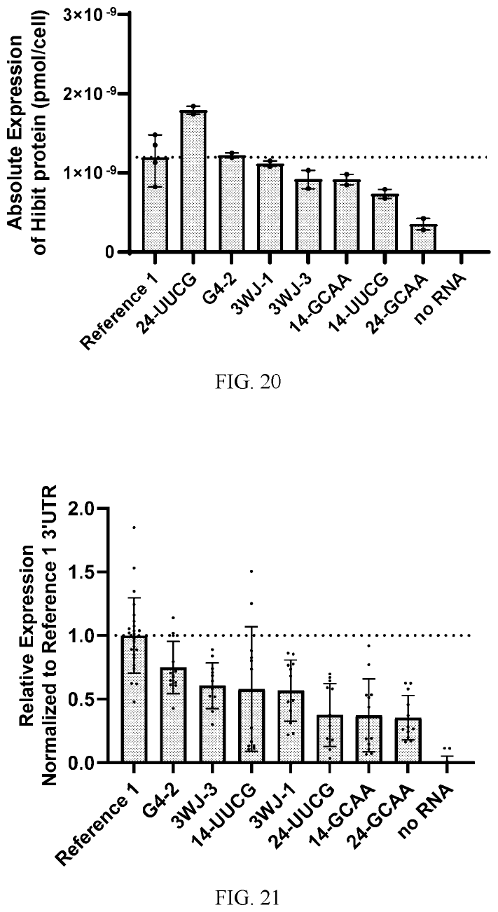

2 μg mRNA encoding HiBiT tagged gene modifying polypeptide and 4 μg template RNA were co-delivered to 0.1 million primary mouse hepatocytes by nucleofection using Lonza's Amaxa™ Nucleofector 96-well Shuttle™. At 24 hours post-nucleofection, the hepatocytes were lysed and analyzed for HiBiT expression using Promega's Nano-Glo® HiBiT Lytic Detection System. The Absolute Expression of HiBiT protein (pmol/cell) was calculated using a standard HiBiT quantity ladder prepared using the HiBiT reference protein from Promega (catalog #N3010) and the cell number ladder prepared with ThermoFisher's PrestoBlue™ Cell Viability Reagent (Catalog number: A13261). The results show that several 3′ UTRs, such as 24-UUCG, G4-2, and 3WJ-1 enabled higher or comparable expression of gene modifying polypeptide than the Reference 1 3′ UTR (FIG. 20).

The expression of the HiBiT-tagged gene modifying polypeptide from mRNAs was also examined in hepatocytes from freshly harvested wild-type C57BL/6 mice 24 hours post-nucleofection. The results show that some 3′ UTRs, for example G4-2, enabled comparable expression of gene modifying polypeptide compared to the mRNA comprising Reference 1 3′ UTR (FIG. 21).

Taking these results together, the tested 3′ UTRs were capable of enabling expression of an exemplary gene modifying polypeptide in primary hepatocytes from two different sources, and some 3′ UTRs enabled comparable or higher expression than the Reference 1 3′UTR.

### Example 7: Evaluating Expression of Exemplary Gene Modifying Polypeptide from mRNAs with Modified polyA Tails and Gene Modifying Activity in U2OS Cells

This example describes the use of exemplary gene modifying systems containing a template RNA and an mRNA encoding a gene modifying polypeptide and comprising modified poly A tails to quantify the gene modifying activity and expression of the gene modifying polypeptide in a U2OS cell line.

In this example, an mRNA contained the following segments:
(1) a 5′ cap;
(2) a 5′ UTR having the nucleotide sequence of SEQ ID NO:8259;
(3) a coding sequence (CDS) encoding a gene modifying polypeptide fused to a HiBIT protein tag having the coding nucleotide sequence of SEQ ID NO:8255, which was added to the carboxy-terminal end of the gene modifying polypeptide;
(4) a 3′ UTR having the nucleotide sequence of SEQ ID NO: 8317; and
(5) a poly A tail listed in Table 5 or Table 6.

In this example, the gene modifying polypeptide encoded by the mRNA contained:
(1) an endonuclease and/or DNA binding domain;
(2) a peptide linker; and
(3) a reverse transcriptase (RT) domain.

The gene modifying polypeptide was encoded by mRNA RNAV209 and comprised the amino acid sequence of SEQ ID NO:8257.

In this example, the template RNA co-delivered with the mRNA is RNACS7570 (SEQ ID NO: 8258), comprising the following nucleotide sequence: mG*mC*mC*rGrArArGrCrArCrUrGrCrArCrGrCrCrGrUrGrUrUrUrUrArGrAmGmCmUm AmGmAmAmAmUmAmGmCrArArGrUrUrArArArArUrArArGrGrCrUrArGrUrCrCrGrUr UrArUrCrAmAmCmUmUmGmAmAmAmAmAmGmUmGmGmCmAmCmCmGmAmGm UmCmGmGmUmGmCrArCrCrCrUrGrArCrGrUrArCrGrGrCrGrUrGrCrArGrUrG*mC*mU*mU. Nucleotide modifications are noted as follows: phosphorothioate linkages denoted by an asterisk, 2′-O-methyl groups denoted by an ‘m’ preceding a nucleotide, ribonucleotide denoted by an ‘r’ preceding the nucleotide.

TABLE 5
Nucleotide sequences of control polyA tails used in this example

| PolyA | | |
|---|---|---|
| tail | | SEQ ID |
| name | Nucleotide Sequence | NO |
| Control- | AAAAAAAAAAAAAAAAAAAAAAAAAAAAAAGCAUAUGACUAAAAAAAAAA | 8315 |
| 30L70 | AAAAAAAAAAAAAAAAAAAAAAAAAAAAAAAAAAAAAAAAAAAAAAAAAA | |
| | AAAAAAAAAA | |
| Control- | AAAAAAAAAAAAAAAAAAAAAAAAAAAAAAAAAAAAAAAAAAAAAAAAAA | 8316 |
| 80A | AAAAAAAAAAAAAAAAAAAAAAAAAAAAAA | |

TABLE 6
Nucleotide sequences of modified polyA tails used in this example

| PolyA | | |
|---|---|---|
| tail | | SEQ ID |
| Identifier | Nucleotide Sequence | NO |
| T01 | AAAAAAAAAAAAAAAAAAAAAAAAAAAAAAGGAAAAAAAAAAAAAAAA | 8282 |
| | AAAAAAAAAAAAAAGGGAAAAAAAAAAAAAAAAAAAAAAAAAAAAAAG | |
| | GGGAAAAAAAAAAAAAAAAAAAAAAAAAAAAAA | |
| T02 | AAAAAAAAAAAAAAAAAAAAAAAAAAAAAACCAAAAAAAAAAAAAAAA | 8283 |
| | AAAAAAAAAAAAAACCCAAAAAAAAAAAAAAAAAAAAAAAAAAAAAAC | |
| | CCCAAAAAAAAAAAAAAAAAAAAAAAAAAAAAA | |
| T03 | AAAAAAAAAAAAAAAAAAAAAAAAAAAAAAUUAAAAAAAAAAAAAAAA | 8284 |
| | AAAAAAAAAAAAAAUUUAAAAAAAAAAAAAAAAAAAAAAAAAAAAAAU | |
| | UUUAAAAAAAAAAAAAAAAAAAAAAAAAAAAAA | |
| T04 | AAAAAAAAAAAAAAAAAAAAAAAAAAAAAAGGGGAAAAAAAAAAAAAA | 8285 |
| | AAAAAAAAAAAAAAAAGGGGGGAAAAAAAAAAAAAAAAAAAAAAAAAA | |
| | AAAAAGGGGGGGGAAAAAAAAAAAAAAAAAAAAAAAAAAAAAA | |
| T05 | AAAAAAAAAAAAAAAAAAAAAAAAAAAAAACCCCAAAAAAAAAAAAAA | 8286 |
| | AAAAAAAAAAAAAAAACCCCCCAAAAAAAAAAAAAAAAAAAAAAAAAA | |
| | AAAACCCCCCCCAAAAAAAAAAAAAAAAAAAAAAAAAAAAAA | |
| T06 | AAAAAAAAAAAAAAAAAAAAAAAAAAAAAAUUUUAAAAAAAAAAAAAA | 8287 |
| | AAAAAAAAAAAAAAAAUUUUUUAAAAAAAAAAAAAAAAAAAAAAAAAA | |
| | AAAAUUUUUUUUAAAAAAAAAAAAAAAAAAAAAAAAAAAAAA | |
| T07 | AAAAAAAAAAAAAAAAAAAAAAAAAAAAAAGCCCAAAAAAAAAAAAAA | 8288 |
| | AAAAAAAAAAAAAAAACCCGCCAAAAAAAAAAAAAAAAAAAAAAAAAA | |
| | AAAACGCCGCCGAAAAAAAAAAAAAAAAAAAAAAAAAAAAAA | |
| T08 | AAAAAAAAAAAAAAAAAAAAAAAAAAAAAGGUGAAAAAAAAAAAAAAA | 8289 |
| | AAAAAAAAAAAAAAAAUUGUGUAAAAAAAAAAAAAAAAAAAAAAAAAA | |
| | AAAAGUUGUGUUAAAAAAAAAAAAAAAAAAAAAAAAAAAAAA | |
| T09 | AAAAAAAAAAAAAAAAAAAAAAAAAAAAAUUCCAAAAAAAAAAAAAAA | 8290 |
| | AAAAAAAAAAAAAAACUUCUCAAAAAAAAAAAAAAAAAAAAAAAAAAA | |
| | AAAUCUCUCUUAAAAAAAAAAAAAAAAAAAAAAAAAAAAAA | |
| T10 | AAAAAAAAAAAAAAAAAAAAAAAAAAAAAAGUUCAAAAAAAAAAAAAA | 8291 |
| | AAAAAAAAAAAAAAAAUCUUCCAAAAAAAAAAAAAAAAAAAAAAAAAA | |
| | AAAACCGUCGCGAAAAAAAAAAAAAAAAAAAAAAAAAAAAAA | |
| T11 | AAAAAAAAAAAAAAAGGAAAAAAAAAAAAAAAAAAAAAAAAAGGAAAA | 8292 |
| | AAAAAAAAAAAAAAAAAAAAAAAAAAAAAAAGGAAAAAAAAAAAAAAA | |
| | AAAAAAAAAAAAAAAAAAAAAAAAAAAAAA | |
| T12 | AAAAAAAAAAAAAAACCAAAAAAAAAAAAAAAAAAAAAAAAACCAAAA | 8293 |
| | AAAAAAAAAAAAAAAAAAAAAAAAAAAAAACCCAAAAAAAAAAAAAAA | |
| | AAAAAAAAAAAAAAAAAAAAAAAAAAAAAA | |
| T13 | AAAAAAAAAAAAAAAUUAAAAAAAAAAAAAAAAAAAAAAAAAUUAAAA | 8294 |
| | AAAAAAAAAAAAAAAAAAAAAAAAAAAAAAAUUAAAAAAAAAAAAAAA | |
| | AAAAAAAAAAAAAAAAAAAAAAAAAAAAAA | |
| T14 | AAAAAAAAAAAAAAAGGGGAAAAAAAAAAAAAAAAAAAAAAAAAGGGG | 8295 |
| | AAAAAAAAAAAAAAAAAAAAAAAAAAAAAAAAAAAGGGGAAAAAAAAA | |
| | AAAAAAAAAAAAAAAAAAAAAAAAAAAAAAAAAAAA | |
| T15 | AAAAAAAAAAAAAAACCCCAAAAAAAAAAAAAAAAAAAAAAAAACCCC | 8296 |
| | AAAAAAAAAAAAAAAAAAAAAAAAAAAAAAAAACCCCCAAAAAAAAAA | |
| | AAAAAAAAAAAAAAAAAAAAAAAAAAAAAAAAAAA | |
| T16 | AAAAAAAAAAAAAAAUUUUAAAAAAAAAAAAAAAAAAAAAAAAAUUUU | 8297 |
| | AAAAAAAAAAAAAAAAAAAAAAAAAAAAAAAAAAAUUUUAAAAAAAAA | |
| | AAAAAAAAAAAAAAAAAAAAAAAAAAAAAAAAAAAA | |
| T17 | AAAAAAAAAAAAAAAGGGGGGGGAAAAAAAAAAAAAAAAAAAAAAAAA | 8298 |
| | GGGGGGGGAAAAAAAAAAAAAAAAAAAAAAAAAAAAAAAAAAAGGGGG | |
| | GGGAAAAAAAAAAAAAAAAAAAAAAAAAAAAAAAAAAAAAAAAAAAAA | |
| T18 | AAAAAAAAAAAAAAACCCCCCCCAAAAAAAAAAAAAAAAAAAAAAAAAC | 8299 |
| | CCCCCCCAAAAAAAAAAAAAAAAAAAAAAAAAAAAAAAAAACCCCCCCC | |
| | CAAAAAAAAAAAAAAAAAAAAAAAAAAAAAAAAAAAAAAAAAAAAA | |
| T19 | AAAAAAAAAAAAAAAUUUUUUUUAAAAAAAAAAAAAAAAAAAAAAAAA | 8300 |
| | UUUUUUUUAAAAAAAAAAAAAAAAAAAAAAAAAAAAAAAAAAAUUUUU | |
| | UUUAAAAAAAAAAAAAAAAAAAAAAAAAAAAAAAAAAAAAAAAAAAAA | |
| T20 | AAAAAAAAAAAAAAACCGCCCCGAAAAAAAAAAAAAAAAAAAAAAAAA | 8301 |
| | GCCCGCCCAAAAAAAAAAAAAAAAAAAAAAAAAAAAAAAAAAACCCCGC | |
| | CGAAAAAAAAAAAAAAAAAAAAAAAAAAAAAAAAAAAAAAAAAAAAA | |
| T21 | AAAAAAAAAAAAAAAGUUGUGUUAAAAAAAAAAAAAAAAAAAAAAAAA | 8302 |
| | GUUGUGGUAAAAAAAAAAAAAAAAAAAAAAAAAAAAAAAAAAAGGUUG | |
| | UGGAAAAAAAAAAAAAAAAAAAAAAAAAAAAAAAAAAAAAAAAAAAAA | |
| T22 | AAAAAAAAAAAAAAAUCCUCUCCAAAAAAAAAAAAAAAAAAAAAAAAA | 8303 |
| | UUCCUUCUAAAAAAAAAAAAAAAAAAAAAAAAAAAAAAAAACCUCCUU | |
| | CUAAAAAAAAAAAAAAAAAAAAAAAAAAAAAAAAAAAAAAAAAAAAA | |
| T23 | AAAAAAAAAAAAAAACGUCGUCUAAAAAAAAAAAAAAAAAAAAAAAAA | 8304 |
| | CUGUCGCUAAAAAAAAAAAAAAAAAAAAAAAAAAAAAAAAAAACCUGU | |
| | CCUAAAAAAAAAAAAAAAAAAAAAAAAAAAAAAAAAAAAAAAAAAAAA | |
| T24 | AAAAAAAAAAAAAAAGGAAAAAAAAAAAAAAAAAAAAAAAAAGGGAAA | 8305 |
| | AAAAAAAAAAAAAAAAAAAAAAAAAAAAAAAAGGGGAAAAAAAAAAAA | |
| | AAAAAAAAAAAAAAAAAAAAAAAAAAAAAAAAA | |
| T25 | AAAAAAAAAAAAAAACCAAAAAAAAAAAAAAAAAAAAAAAAACCCAAA | 8306 |
| | AAAAAAAAAAAAAAAAAAAAAAAAAAAAAAACCCCAAAAAAAAAAAAA | |
| | AAAAAAAAAAAAAAAAAAAAAAAAAAAAAAAAA | |
| T26 | AAAAAAAAAAAAAAAUUAAAAAAAAAAAAAAAAAAAAAAAAAUUUAAA | 8307 |
| | AAAAAAAAAAAAAAAAAAAAAAAAAAAAAAAUUUUUAAAAAAAAAAAA | |
| | AAAAAAAAAAAAAAAAAAAAAAAAAAAAAAAAA | |
| T27 | AAAAAAAAAAAAAAAGGGGAAAAAAAAAAAAAAAAAAAAAAAAAGGGG | 8308 |
| | GGAAAAAAAAAAAAAAAAAAAAAAAAAAAAAAAAAAAGGGGGGGGAAA | |
| | AAAAAAAAAAAAAAAAAAAAAAAAAAAAAAAAAAAAAAAAAA | |
| T28 | AAAAAAAAAAAAAAACCCCAAAAAAAAAAAAAAAAAAAAAAAAACCCCC | 8309 |
| | CAAAAAAAAAAAAAAAAAAAAAAAAAAAAAAAAAACCCCCCCCCAAAAA | |
| | AAAAAAAAAAAAAAAAAAAAAAAAAAAAAAAAAAAAAAAA | |
| T29 | AAAAAAAAAAAAAAAUUUUAAAAAAAAAAAAAAAAAAAAAAAAAUUUU | 8310 |
| | UUAAAAAAAAAAAAAAAAAAAAAAAAAAAAAAAAAAAUUUUUUUUUAA | |
| | AAAAAAAAAAAAAAAAAAAAAAAAAAAAAAAAAAAAAAAAAA | |
| T30 | AAAAAAAAAAAAAAACCCGAAAAAAAAAAAAAAAAAAAAAAAAACCCG | 8311 |
| | CCAAAAAAAAAAAAAAAAAAAAAAAAAAAAAAAAAAAGCCGCCCGAAA | |
| | AAAAAAAAAAAAAAAAAAAAAAAAAAAAAAAAAAAAAAAAAA | |
| T31 | AAAAAAAAAAAAAAAGGUGAAAAAAAAAAAAAAAAAAAAAAAAAUUGU | 8312 |
| | GUAAAAAAAAAAAAAAAAAAAAAAAAAAAAAAAAAAAGUGGUUGUAAA | |
| | AAAAAAAAAAAAAAAAAAAAAAAAAAAAAAAAAAAAAAAAAA | |
| T32 | AAAAAAAAAAAAAAACUCUAAAAAAAAAAAAAAAAAAAAAAAAACCUU | 8314 |
| | CCAAAAAAAAAAAAAAAAAAAAAAAAAAAAAAAAAAACUUCUUCUAAA | |
| | AAAAAAAAAAAAAAAAAAAAAAAAAAAAAAAAAAAAAAAAAA | |
| T33 | AAAAAAAAAAAAAAACCUGAAAAAAAAAAAAAAAAAAAAAAAAAGUCC | 8315 |
| | CGAAAAAAAAAAAAAAAAAAAAAAAAAAAAAAAAAAAUUGCGCUGAAA | |
| | AAAAAAAAAAAAAAAAAAAAAAAAAAAAAAAAAAAAAAAAAA | |

To evaluate the expression level of the gene modifying polypeptide from mRNAs with modified poly A tails, a landing pad cell line called U2OS-BFP was used. U2OS-BFP was an engineered U2OS cell line that stably expressed Blue Fluorescence Protein (BFP) from its genome.

0.25 μg mRNA encoding the HiBiT tagged gene modifying polypeptide and 2 μg template RNA were co-delivered to 0.25 million U2OS-BFP cells by nucleofection using Lonza's Amaxa™ Nucleofector 96-well Shuttle™. At 6 hours post-nucleofection, the cells were lysed and analyzed for HiBiT expression using Promega's Nano-Glo® HiBiT Lytic Detection System.

As indicated in FIG. 1, 18 out of the total 31 modified poly A tails enabled comparable or higher expression of the gene modifying polypeptide than the mRNA with the control 80A tail. Several of the modified poly A tails, such as T26, T28, T25, T30, T33, T27, T24, T16, and T18, increased the expression of the gene modifying polypeptide dramatically, up to four-fold relative to the control 80A tail. Without wishing to be bound by theory, the results indicated that intervening non-adenosine nucleotides in the middle of a poly A tail can enhance the protein expression activity of an mRNA. The results further suggest that intervening cytosine or uridine nucleotides may provide superior enhancement to protein expression than intervening guanosine nucleotides. The results further suggest that modified poly As following the pattern 15A-25A-35A-45A (with hyphens indicating intervening non-adenosine nucleotides) or similar provides superior enhancement to protein expression than modified poly As following a more even pattern such as 30A-30A-30A-30A. Without wishing to be bound by theory, a higher level of expression of gene modifying polypeptide is thought to increase the targeted gene modifying activity of the system.

U2OS-BFP cells were used to evaluate the effects of the poly A tails on targeted gene modification (e.g., via changes in expression of gene modifying polypeptide). The template RNA was designed to convert the DNA sequence in the genome encoding BFP into that encoding GFP. The percentage of GFP positive cells (GFP %) among the harvested U2OS-BFP cell sample was used to assess the gene modifying activities of the system. In each reaction, the template RNA and gene modifying polypeptides were invariant as the poly A tail of the mRNA was varied.

0.25 μg of the mRNAs and 2 μg template RNA RNACS7570 (SEQ ID NO: 8258) were co-delivered on day 0 by nucleofection using Lonza's Amaxa™ Nucleofector 96-well Shuttle™ to 0.25 million U2OS-BFP cells. On day 4, the cells were harvested and examined by flow cytometry for BFP and GFP expression. FlowJo was used to analyze the data and determine % GFP positive cells. GraphPad Prism was used to calculate significance and correlation.

Before normalization, U2OS-BFP cells nucleofected with mRNA comprising Control-80A poly A tails showed a % GFP positive rate of 68% on day 4. Many of the cell samples nucleofected with mRNA comprising different modified poly A tails exhibited significant amounts of GFP positive cells. Samples nucleofected with 22 different modified poly A tails exhibited a comparable or higher GFP % than the control as the best performing poly A tail, T26 (SEQ ID NO: 8307) exhibited 88% GFP+ rate. The results show that many of the tested modified poly A tails successfully facilitated expression of gene modifying polypeptide sufficient for gene editing in U2OS cells. The results further show that 22 modified poly A tails were particularly high performing and facilitated higher gene modifying activities than the Control-80A poly A tail in U2OS-BFP cells. Additionally, the expression enhancement provided by intervening cytosine or uridine nucleotides may facilitate superior enhancement to gene modifying activities than the expression enhancement provided by intervening guanosine nucleotides. The results further suggest that the expression enhancement provided by modified poly As following the pattern 15A-25A-35A-45A (with hyphens indicating intervening non-adenosine nucleotides) or similar may facilitate superior enhancement to gene modifying activities than the expression enhancement provided by modified poly As following a more even pattern such as 30A-30A-30A-30A.

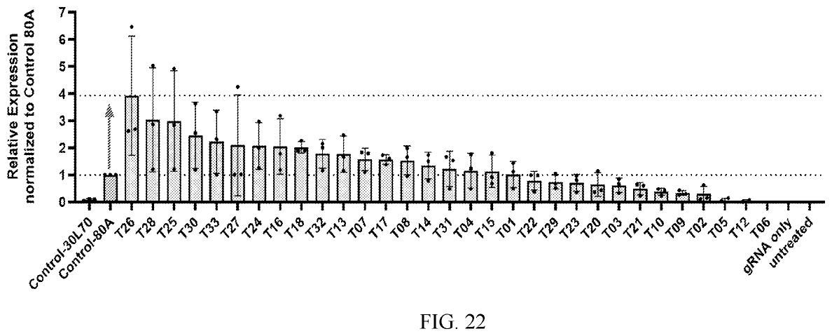

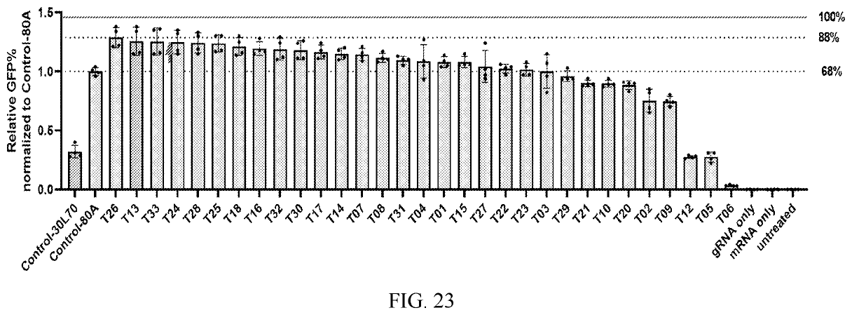

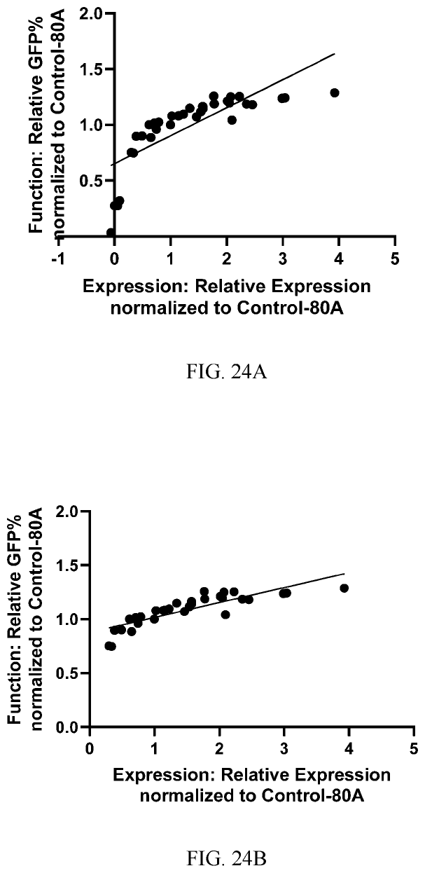

The data from FIG. 22 and FIG. 23 was analyzed by correlation analysis (FIGS. 24A-24B). The correlation analysis of FIGS. 24A-B suggests that, for most mRNAs with modified poly A tails, the expression level of the gene modifying polypeptide was well-correlated with the gene modifying function. Accordingly, improvements in gene modifying function can likely be attributed to stronger expression of the gene modifying polypeptide from the mRNAs with these novel modified poly A tails. The results suggest that the editing activity of gene modifying systems comprising mRNA encoding gene modifying polypeptides can be improved using poly A tails that increase gene modifying polypeptide expression as described herein.

### Example 8: Evaluating Expression of Exemplary Gene Modifying Polypeptide from mRNAs with Modified polyA Tails and Gene Modifying Activity in Primary Mouse Hepatocytes

This example describes the use of exemplary gene modifying systems containing a template RNA and an mRNA encoding a gene modifying polypeptide and comprising modified poly A tails to quantify the gene modifying activity and expression of the gene modifying polypeptide in primary mouse hepatocytes.

In this example, an mRNA contained the following segments:
(1) a 5′ cap;
(2) a 5′ UTR having the nucleotide sequence of SEQ ID NO: 8259:
(3) a coding sequence (CDS) encoding a gene modifying polypeptide fused to a HiBiT protein tag having the coding nucleotide sequence of SEQ ID NO: 8255, which was added to the carboxy-terminal end of the gene modifying polypeptide;
(4) a 3′ UTR having the nucleotide sequence of SEQ ID NO: 8317; and
(5) a poly A tail listed in Table 5 or Table 6.

In this example, the gene modifying polypeptide encoded by the mRNA contained:
(1) an endonuclease and/or DNA binding domain;
(2) a peptide linker; and
(3) a reverse transcriptase (RT) domain.

The gene modifying polypeptide was the same as that described in Example 1.

In this example, the gene modifying system comprised an mRNA encoding the gene modifying polypeptide and either of two template RNAs. Template RNA A was RNACS229 (SEQ ID NO:8320):

mU*mC*mA*rGrArGrGrArArGrCrUrGrGrGrCrCrArCrCrGrUrU
rUrUrArGrAmGmCmUmAmGmAmAmAmUmAmGmCrArArGrUrUrArAr
ArArUrArArGrGrCrUrArGrUrCrCrGrUrUrArUrCrAmAmCmUmU
mGmAmAmAmAmAmGmUmGmGmCmAmCmCmGmAmGmUmCmGmGmUmGmCr
UrGrGrArGrCrArGrUrArArUrGrGrCrUrGrGrUrGrGrCrCrCrA
rGrC*mU*mU*mC.

Template RNA B was RNACS1515 (SEQ ID NO: 8321), comprising the following nucleotide sequence:

mU*mU*mA*rCrCrArArCrUrUrUrCrUrCrCrArUrGrGrCrGrUrU
rUrUrArGrAmGmCmUmAmGmAmAmAmUmAmGmCrArArGrUrUrArAr
ArArUrArArGrGrCrUrArGrUrCrCrGrUrUrArUrCrAmAmCmUmU
mGmAmAmAmAmAmGmUmGmGmCmAmCmCmGmAmGmUmCmGmGmUmGmCm
U*mU*mU*mU.

For expression analysis, template RNA RNACS7570 (given in Example 1), was co-delivered with the mRNA.

To evaluate expression and gene modifying activity associated with modified poly A tails further in primary cells, primary mouse hepatocytes freshly harvested from wild-type C57BL/6 mice were used. In each gene modifying system used on a primary cell, the template RNAs and all segments of the mRNA encoding the gene modifying polypeptide remained invariant, except the poly A tail.

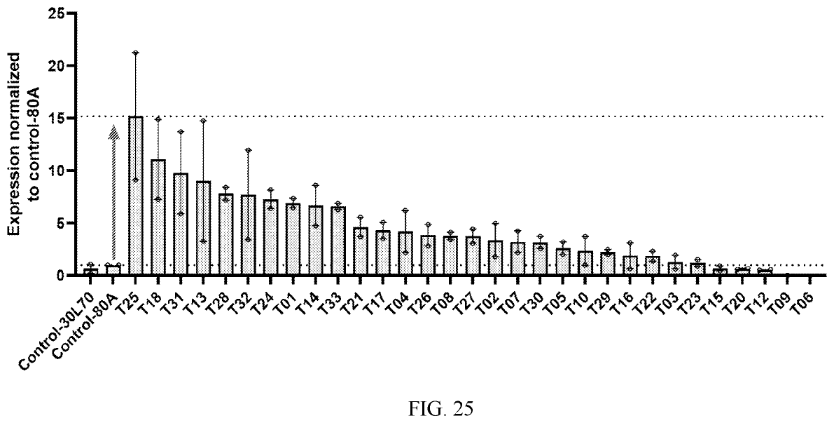

To evaluate expression of the gene modifying polypeptide at 18 hours post-nucleofection, 2 μg mRNA and 4 μg template RNA RNACS7570 (SEQ ID NO:58) were co-delivered to 0.1 million primary mouse hepatocytes by nucleofection using Lonza's Amaxa™ Nucleofector 96-well Shuttle™. At 18 hours post-nucleofection, the hepatocytes were lysed and analyzed for HiBiT expression using Promega's Nano-Glo® HiBiT Lytic Detection System. 24 out of 31 modified poly A tails tested enabled 2-fold or higher expression of gene modifying polypeptide compared to Control-80A tail (FIG. 25). The highest performing modified poly A tail, T25, dramatically increased the gene modifying polypeptide expression approximately 15-fold comparing to Control-80A tail. The results suggest that many of the modified poly A tails, e.g., T25, T18, T31, T13, T28, T32, T24, T01, T14, T33, T21, T17, T04, T26, T08, T27, T02, T07, T30, T05, T10, T29, T16, and T22, successfully enabled the expression of gene modifying polypeptide in primary mouse hepatocytes. The results highlighted that the intervening non-adenosine nucleotides in the middle of a polyA tail greatly enhanced the mRNA expression in primary cells, consistent with the expression enhancement described in U2OS-BFP cells described above.

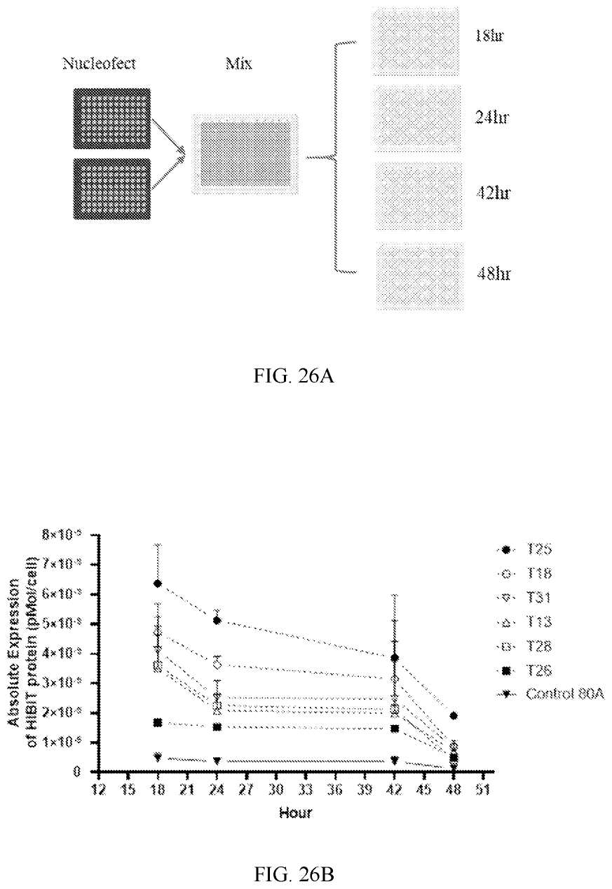

An experiment was conducted evaluating the expression of gene modifying polypeptide from the mRNAs over the time course of 18 hours to 48 hours post-nucleofection (FIG. 26A). 2 μg mRNA and 4 μg template RNA RNACS7570 (SEQ ID NO:8240) were co-delivered to 0.1 million primary mouse hepatocytes by nucleofection using Lonza's Amaxa™ Nucleofector 96-well Shuttle™. Two duplicate sets of all reactions were prepared in two 96-well plate and nucleofected, followed by mixing of identical reactions in one 96-well deep-well plate, resulting 0.2 million nucleofected hepatocytes for each reaction condition. These hepatocytes were then split into four 96-well plates and cultured at 37° C. for 18 hours, 24 hours, 42 hours, and 48 hours, respectively. At each timepoint, one of the four plates was moved to −80° C. for storage. At the time of analysis, the hepatocytes in all four plates were thawed, lysed and examined for HiBiT expression using Promega's Nano-Glo® HiBiT Lytic Detection System.

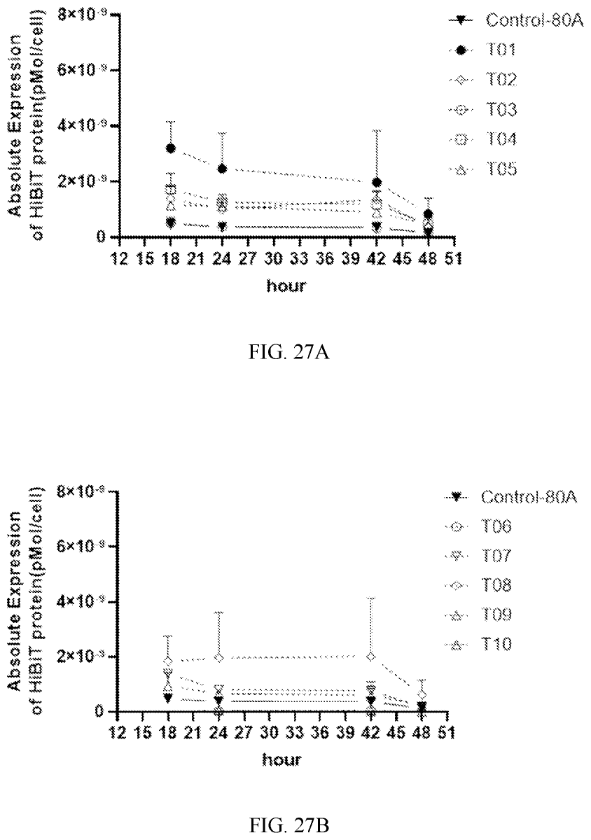

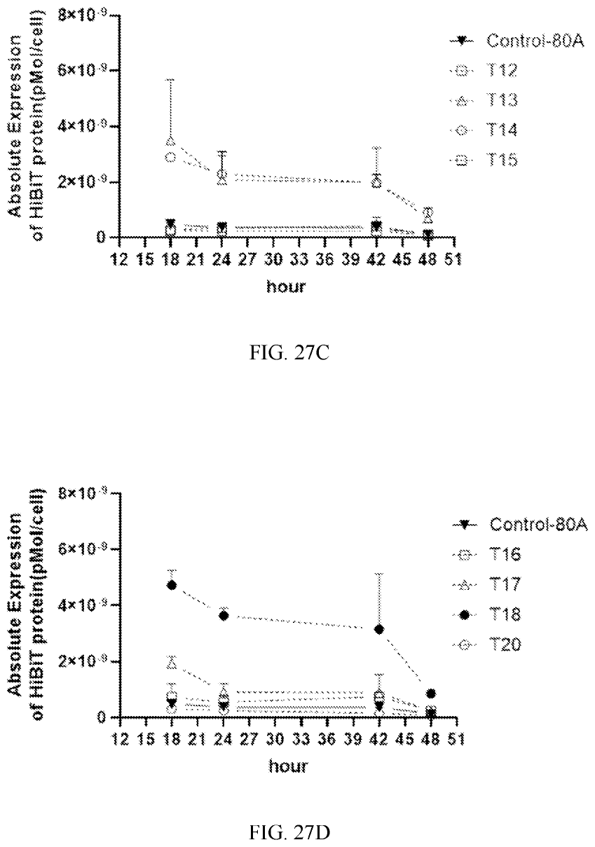

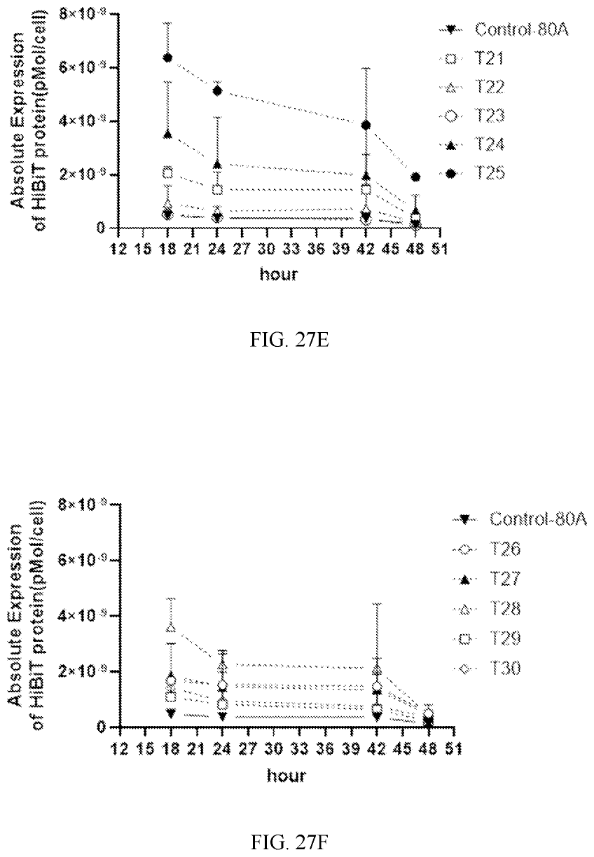

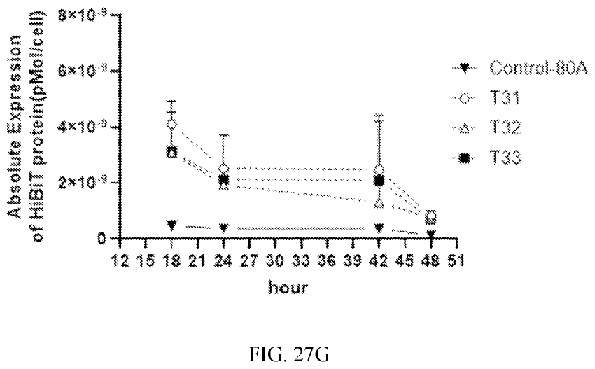

The data showed that nucleofection of primary cells with mRNAs comprising many of the modified poly A tails resulted in sharply increased expression of gene modifying polypeptide relative to that provided by control mRNAs and that the increase was maintained throughout the time period of the test, with expression beginning at its highest point at 18 hours post-nucleofection and generally decreased over time (FIG. 26B). In particular, the highest performing modified poly A tail, T25, was associated with an approximately 13.5-fold increase in gene modifying polypeptide expression relative to Control-80A at 18 hours post-nucleofection, and that the increase over Control-80A was 13.7-fold at 24 hours post-nucleofection, 10.6-fold at 42 hours post-nucleofection, and 15.0-fold at 48 hours post-nucleofection (FIG. 26B). The expression profiles for all mRNAs used in this example are shown in FIGS. 27A-27G.

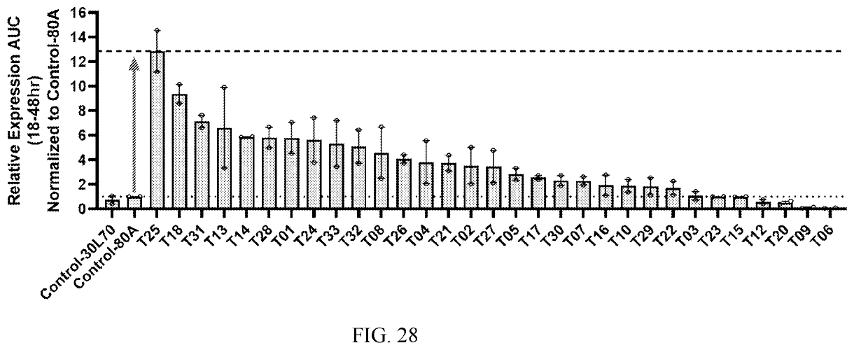

Using the expression profiles from 18 hours to 48 hours obtained above from each tested mRNA, the Area Under the Curve (AUC) was calculated in GraphPad Prism. The results show that nucleofection of many of the mRNAs with modified poly A tails enabled dramatic increases in the gene modifying polypeptide expression AUC relative to the AUC from mRNA containing Control-80A poly A tail (FIG. 28). The results show that modified poly A tail T25 enabled a 13-fold increase in AUC; modified poly A tail T18 enabled a 9-fold increase; modified poly A tail T31 enabled a 7-fold increase; four modified poly A tails (T13, T14, T28, and T01) enabled a 6-fold increase; three modified poly A tails (T24, T33, and T32) enabled a 5-fold increase; four modified poly A tails (T08, T26, T04, and T21) enabled a 4-fold increase; four modified poly A tails (T02, T27, 05, and T17) enabled a 3-fold increase; six modified poly A tails (T30, T07, T16, T10, T29, and T22) enabled a 2-fold increase; and three modified poly A tails (T03, T23, T15) enabled a comparable AUC (all comparisons relative to AUC of Control-80A poly A tail mRNA). The up-to-13-fold increase in the expression AUC over the control polyA tail demonstrated that these modified poly A tails are capable of increasing expression of exemplary gene modifying peptide from mRNA.

Together, these results further suggest that intervening cytosine or uridine nucleotides may provide superior enhancement to protein expression than intervening guanosine nucleotides. Together the results further suggest that modified poly As following the pattern 15A-25A-35A-45A (with hyphens indicating intervening non-adenosine nucleotides) or similar provides superior enhancement to protein expression than modified poly As following a more even pattern such as 30A-30A-30A-30A.

To evaluate the capacity of the mRNAs encoding gene modifying polypeptides and comprising modified poly A tails for inducing targeted gene modification in primary cells, a gene modifying system, consisting of either of two exemplary template RNAs and one mRNA encoding the gene modifying polypeptide and comprising a modified poly A tail, was co-delivered to primary mouse hepatocytes freshly harvested from wild-type C57B/L6 mice. The two template RNAs targeted gene modification in the wild-type mouse Fah gene, converting the wild-type sequence (GGAGCGGTAATGCCTGGTGG; SEQ ID NO: 8318) to the mutated disease sequence observed in the mouse model of Tyrosinemia (GGAGCAGTAATGGCTGGTGG; SEQ ID NO: 8319); underlines indicate the modified nucleotides. In each reaction, two template RNAs and all segments of mRNA, except the poly A tail remained invariant.

1 μg mRNA, 4 μg template RNA A, and 5 μg template RNA B were co-delivered on day 0 by nucleofection using Lonza's Amaxa™ Nucleofector 96-well Shuttle™ to 0.1 million primary mouse hepatocytes by nucleofection using Lonza's Amaxa™ Nucleofector 96-well Shuttle™. The hepatocytes were harvested on day 5, followed by extraction of genomic DNA (gDNA). The gDNA samples were submitted for Targeted Amplicon Sequencing which counted the copy number of wild-type unmodified sequence and the number with the intended modification in the region of interest in the mFah gene. Modification % of the mutation installation was calculated by dividing the copy number of gDNA with the intended modification by the total gDNA copy number in the sample and multiplying by 100%.

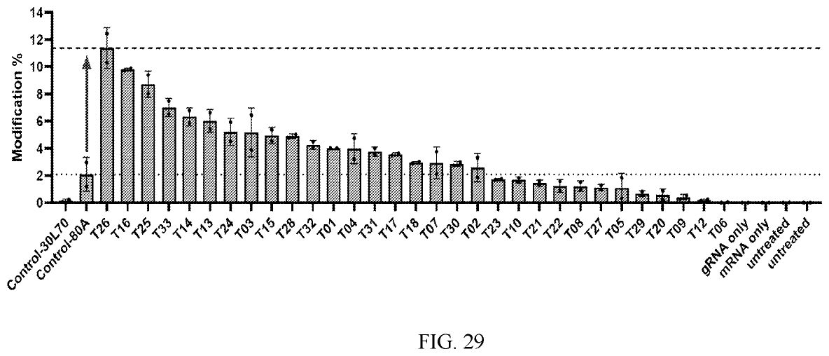

The results show that many mRNAs with modified polyA tails enabled dramatically higher modification % in primary mouse hepatocytes compared to mRNAs comprising Control 80A polyA (which yielded a 2% modification) (FIG. 29). For example, T26 tail enabled 11% modification; T16 tail enabled 10% modification; T25 tail enabled 9% modification; T33 tail enabled 7% modification; T14 and T13 tails enabled 6% modification; T24, T03, T15, and T28 tails enabled 5% modification; T32, T01, T04, T31, and T17 tails enabled 4% modification; T18, T07, T30, and T02 tails enabled 3% modification; and T23 and T10 tails enabled 2% modification. The results show that use of modified poly As in mRNAs encoding an exemplary gene modifying polypeptide resulted in up to ~5 fold improvement in modification percent (up to 11% modification) relative to Control 80A-containing mRNAs, and demonstrate that modified poly As can result in improved efficacy of gene modifying systems in primary cells. The results further suggest that the expression enhancement provided by intervening cytosine or uridine nucleotides may facilitate superior enhancement to gene modifying activities than the expression enhancement provided by intervening guanosine nucleotides. The results also suggest that the expression enhancement provided by modified poly As following the pattern 15A-25A-35A-45A (with hyphens indicating intervening non-adenosine nucleotides) or similar may facilitate superior enhancement to gene modifying activities than the expression enhancement provided by modified poly As following a more even pattern such as 30A-30A-30A-30A.

### Example 9: Evaluating Expression of Exemplary Gene Modifying Polypeptide from mRNAs Equipped with UTRs and polyA Tails in Primary Human T Cells

This example evaluates the expression of gene-modifying polypeptide in primary human T cells from mRNAs equipped with exemplary UTRs and an exemplary poly A tail.

In this example, an mRNA contained the following segments:
(1) a 5′ cap;
(2) one of the 5′ UTRs listed in Table 7;
(3) a coding sequence (CDS) encoding a gene modifying polypeptide fused to a HiBIT protein tag having the coding nucleotide sequence of SEQ ID NO:8255;
(4) one of the 3′ UTRs listed in Table 8; and
(5) a poly A tail (T26):

(SEQ ID NO: 8307)
AAAAAAAAAAAAAAAUUAAAAAAAAAAAAAAAAAAAAAAAAAUUUAA
AAAAAAAAAAAAAAAAAAAAAAAAAAAAAAAAUUUUUAAAAAAAAAA
AAAAAAAAAAAAAAAAAAAAAAAAAAAAAAAAAAA

In this example, the gene modifying polypeptide encoded by all the mRNAs was RNAIVT3689 and comprised the amino acid sequence of:

(SEQ ID NO: 8371)
MDEYQRSLSRPLLTIMSINIEGLSLAKEELLAKMSEDISCDILCIQETHRDITMRRPKIL
GMQLAVERPHRQYGSAIFVRSGVAISATSLTEVNNIEILSVELDSCTVSSLYKPPGADF
YFTPPTSCHNHEAHFVVGDFNSHSCVWGYDEDDRNGEAVLTWADNSRMSLLHDSK
LPPSFNSGRWKRGYNPDLIFVKESISHQCTKRVLNPIPNTQHRPICCVAYAAVRPKSV
PFRRRYNFNKANWTKFTETLEAAISDIEPSIENYDLFVEAVKRSSRLSIPRGCRTSYLP
GLNEESLNQLQEYLRLFQENPYSDGTIAAGQKLSTALANAKKDRWIELLENLDMSKS
SRKAWQLLRRLDSDPLVNPGHANVTPDQIAHQLIQNGKTNCSRIKMKINRVPELETH
QLSSPLNLKELREAIKRCKTGKAPGLDDLMMEQIKHLGAKAENWLLKFYNQCLAHK
QIPRAWRKTKIIAILKPGKDASNARNYRPISLLCHLYKVYERMLLNRLGPVIEPKLIAQ
QAGFRPGKNCTGQILHLTEHIEEGYEKGCITGTVFVDLTAAYDTVQHRKMLHKVYHI
TRDFDFTKTVQTLLENRSFYVEFQGQKSRWRRQKNGLPQGSVLAPTLFNIFTNDQPQ
PPLTKSFIYADDLGLTTQAKDFETVEKQLTNALKDLSSYYKENHLKPNPAKTQVCAF
HLRNREANRKLKVTWEGQELEHCFHPKYLGVTLDRTLTYRKHCMNTKHKVAARN
NILRKLTGSAWGADPQVIRTSALALSFSTAEYACPVWHKSAHAKQVDIALNETCRIIT
GCLKPTPVDKLYKLAGIAPPDVRREVAANGERKKVEHCESHPLHGYHPPPTRLKSRK
GFMRTTTPLDVPPAAARVSLWAAKPGNSNWMAPQEGLPPGANQEWATWKSLNRL
RSGVGRSKDNLARWHYLEESSTLCDCGAEQTTQHMYACPQCPASCTEEELFKATDN
AVAVARFWSKTIGGGSPKKKRKVSGSETPGTSESATPESVSGWRLFKKIS

In this example, the template RNA co-delivered with all the mRNAs comprises the nucleotide sequence of:

(SEQ ID NO: 8372)
AGGGGGACACGGAAAGAGCCUCCCCGAAGAUUGAGUGAAUUCAGUCGGG
CGUCCCCUGGGCAACGUUUCUUGUAAGCGGCCGAUCUUUCCACCCCAAA
AGCAUUGGAUGAGUUUACGGAUCCGAAUUCUCGACGGAUCGAUCCGAAC
AAACGACCCAACACCCGUGCGUUUUAUUCUGUCUUUUUAUUGCCGAUCC
CCCGGCCGCUUUACUUGUACAGCUCGUCCAUGCCGAGAGUGAUCCCGGC
GGCGGUCACGAACUCCAGCAGGACCAUGUGAUCGCGCUUCUCGUUGGGG
UCUUUGCUCAGGGCGGACUGGGUGCUCAGGUAGUGGUUGUCGGGCAGCA
GCACGGGGCCGUCGCCGAUGGGGGUGUUCUGCUGGUAGUGGUCGGCGAG
CUGCACGCUGCCGUCCUCGAUGUUGUGGCGGAUCUUGAAGUUCACCUUG
AUGCCGUUCUUCUGCUUGUCGGCCAUGAUAUAGACGUUGUGGCUGUUGU
AGUUGUACUCCAGCUUGUGCCCCAGGAUGUUGCCGUCCUCCUUGAAGUC
GAUGCCCUUCAGCUCGAUGCGGUUCACCAGGGUGUCGCCCUCGAACUUC
ACCUCGGCGCGGGUCUUGUAGUUGCCGUCGUCCUUGAAGAAGAUGGUGC
GCUCCUGGACGUAGCCUUCGGGCAUGGCGGACUUGAAGAAGUCGUGCUG
CUUCAUGUGGUCGGGGUAGCGGCUGAAGCACUGCACGCCGUAGGUCAGG
GUGGUCACGAGGGUGGGCCAGGGCACGGGCAGCUUGCCGGUGGUGCAGA
UGAACUUCAGGGUCAGCUUGCCGUAGGUGGCAUCGCCCUCGCCCUCGCC
GGACACGCUGAACUUGUGGCCGUUUACGUCGCCGUCCAGCUCGACCAGG
AUGGGCACCACCCCGGUGAACAGCUCCUCGCCCUUGCUCACCAUGGUGG
CUUUACCAACAGUACCGGAAUGCCAAGCUUGGGUCCUGUGUUCUGGCGG
CAAACCCGUUGCGAAAAAGAACGUUCACGGCGACUACUGCACUUAUAUA
CGGUUCUCCCCCACCCUCGGGAAAAAGGCGGAGCCAGUACACGACAUCA
CUUUCCCAGUUUACCCCGCGCCACCUUCUCUAGGCACCGGAUCAAUUGC
CGACCCCUCCCCCCAACUUCUCGGGGACUGUGGGCGAUGUGCGCUCUGC
CCUAGUUGCUUGUGAUUUCUUUUCUUUUUUAUUUUAUUUCCAUUAUUUG
AAAUGUAUUUGUUGUAGCAAUGCUUUUGACACGAAAUAAAUAAAAGAAA
AAAAAAAAAAAAAAAAAAAAAAAAAAAAAAAAAAAAAAAAAAAAAAAAA
AAAAAAAAAAAAAAAAAAAAAAAAAAAA

TABLE 7
Nucleotide sequences of 5′ UTRs used in this example

| 5′ UTR | | SEQ |
|---|---|---|
| Identifier | Nucleotide Sequence | ID NO |
| 5′ Ref | AGGAAAUAAGAGAGAAAAGAAGAGUAAGAAGAAAUAU | 8259 |
| | AAGAGCCACC | |
| 50-1c | AGGAUAAUAUACUUACAUACUUACUAAUUAAUACUAAA | 8214 |
| | CUCAACGCCACC | |
| 70-2b | AGGACAACAAUAACUCUAAUAAACAAACGAAUUCUAUU | 8236 |
| | AUAUCACCACAUUAUAUCUCUAAUCUGCCACC | |
| 70-4c | AGGAUCAUCAAACACAUAAUAAUCAAAUAACAACACUA | 8243 |
| | CUCACAAACACUUACAAAUCAAACACGCCACC | |

TABLE 8
Nucleotide sequences of 3′ UTRs used in this example

| 3′ UTR | | SEQ |
|---|---|---|
| Identifier | Nucleotide Sequence | ID NO |
| 3′ Ref | GCUGGAGCCUCGGUGGCCAUGCUUCUUGCCCCUUGGGC | 8373 |
| | CUCCCCCCAGCCCCUCCUCCCCUUCCUGCACCCGUACCC | |
| | CCGUGGUCUUUGAAUAAAGUCUGA | |
| 3WJ-3 | UUGCCAUGUGUAUGUGGGUUUUUUUUUUCCCACAUACU | 8209 |
| | CUGAUGAUCCUUUUUUUUUUGGAUCAUUCAUGGCAA | |
| 14-UUCG | UUGCGUUCGCGCAA | 8201 |

The performance of selected 5′ and 3′ UTRs on mRNAs was compared by evaluating their efficiency to facilitate the expression of an RNA writer protein in primary human T cells. For fast and reliable protein expression analysis, a C-terminus Hibit tag was added to all the constructs to enable Promega's Nano-Glo® HiBiT Lytic Detection System. The number of cells in each well was determined by PrestoBlue™ Cell Viability reagent to normalize Hibit readout. In this example, the coding region and poly A tail of all the mRNAs were invariant as the 5′ UTRs and 3′ UTRs of these mRNA were varied. When the protein expression was compared, a template RNA was included in every reaction and remained invariant.

Cryopreserved primary human T cells were thawed and cultured in the activation medium for three days. On the day of experiment, 0.2 μg of the mRNAs and 1 μg of template RNA were co-delivered by nucleofection to 0.5 million activated human T cells using Lonza's Amaxa Nucleofector 96-well Shuttle™. The cells were harvested and lysed 2, 4, 6, 8, and 24 hours after nucleofection and kept in −80° C. After collecting all the samples from the six time points, the frozen cell lysates were tested using Promega's Nano-Glo® HiBiT Lytic Detection System, following the manufacturer's protocol.

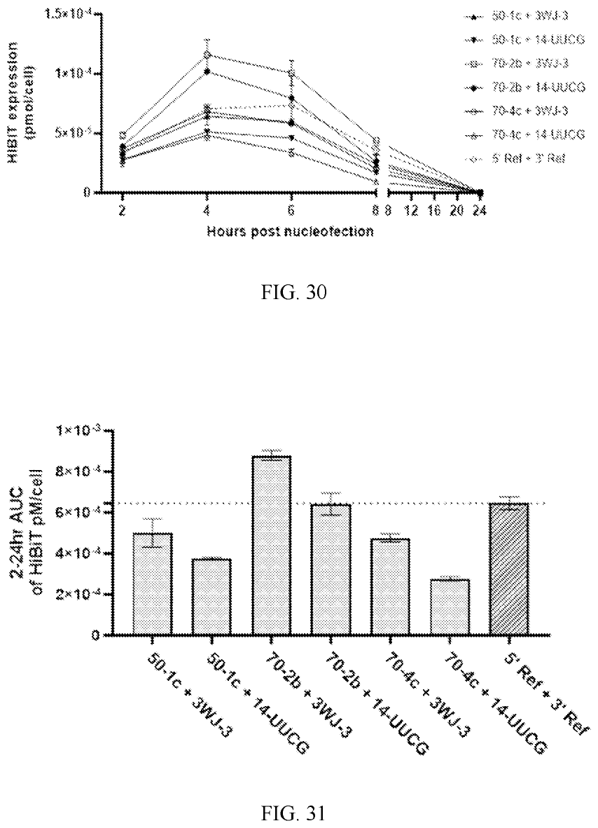

FIG. 30 shows a graph of the protein expression time course at 2, 4, 6, 8, and 24 hours from the mRNAs equipped with test or reference UTRs, analyzed by the Hibit assay. The results show that one mRNA with 70-2b (SEQ ID NO:8236) and 3WJ-3 (SEQ ID NO: 8209) UTRs outperformed the control mRNA with reference UTRs (5′ Ref (SEQ ID NO: 8259)+3′ Ref (SEQ ID NO:8373)), exhibiting higher protein expression at all the time points. Another mRNA with 70-2b (SEQ ID NO:8236) and 14-UUCG (SEQ ID NO:8201) UTRs expresses more protein at early time points (2, 4, and 6 hours) than the control with reference UTRs (5′ Ref+3′ Ref). Two other mRNAs with UTRs 50-1c (SEQ ID NO:8214) and 3WJ-3 (SEQ ID NO:8209), and 70-4c (SEQ ID NO:8243) and 3WJ-3 (SEQ ID NO: 8209), closely trail the performance of the control mRNA (5′ Ref+3′ Ref).

FIG. 31 shows a graph of the protein expression Area Under the Curve (AUC) from 2 to 24 hours calculated from FIG. 30. The results show that one mRNA with 70-2b and 3WJ-3 UTRs outperforms the control mRNA with reference UTRs (5′ Ref+3′ Ref) by having a higher protein expression AUC overall. Another mRNA with UTRs 70-2b and 14-UUCG enables a similar protein expression AUC as the control mRNA (5′ Ref+3′ Ref). Taken together, these results demonstrate that:
(1) UTRs 70-2b and 3WJ-3 perform better than the reference UTRs (5′ Ref+3′ Ref) to enable high protein expression in activated primary human T cells;
(2) UTRs 70-2b and 14-UUCG have a similar efficiency as the reference UTRs (5′ Ref+3′ Ref) to enable high protein expression in activated primary human T cells;
(3) These UTRs and combinations of UTRs are effective to facilitate high protein expression of proteins of interest generally, as well as heterologous gene modifying polypeptides (e.g., as shown in Example 1), and retrotransposon gene modifying polypeptides (e.g., RNAIVT3689 used in this Example).

1. An artificial ribonucleic acid (RNA) molecule for enhancing expression of a polypeptide comprising a reverse transcriptase (RT) domain and optionally an endonuclease domain, the RNA molecule comprising:
(1) a nucleotide sequence encoding the polypeptide,
(2) at least one of:
(a) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269, and 8281; and
(b) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or
(3) a poly(A) tail for enhancing expression of the polypeptide, wherein the poly(A) tail comprises a nucleic acid sequence selected from the group consisting of SEQ ID NOs: 8282-8314.

2-9. (canceled)

10. The artificial RNA molecule of claim 1, wherein the polypeptide is a heterologous gene modifying polypeptide or a retrotransposon gene modifying polypeptide.

11. The artificial RNA molecule of claim 1, wherein the polypeptide comprises the reverse transcriptase domain and the endonuclease domain.

12. The artificial RNA molecule of claim 11, wherein the endonuclease domain is a nickase domain, such as a Cas9 domain selected from SpCas9 domain, a BlatCas9 domain, a Nme2 Cas9 domain, a PnpCas9 domain, a SauCas9 domain, a SauCas9-KKH domain, a SauriCas9 domain, a SauriCas9-KKH domain, a ScaCas9-Sc++ domain, a SpyCas9 domain, a SpyCas9-NG domain, a SpyCas9-SpRY domain, or a St1Cas9 domain, and the reverse transcriptase domain is selected from a retrovirus reverse transcriptase domain.

13. The artificial RNA molecule of claim 12, wherein the Cas9 domain comprising an N670A mutation, an N611A mutation, an N605A mutation, an N580A mutation, an N588A mutation, an N872A mutation, an N863A mutation, an N622A mutation, or an H840A mutation.

14. The artificial RNA molecule of claim 12, wherein the retrovirus reverse transcriptase domain is a gamma retrovirus-derived reverse transcriptase domain, preferably wherein the gamma retrovirus-derived reverse transcriptase domain comprises an amino acid sequence of a reverse-transcriptase domain sequence from a family selected from AVIRE, BAEVM, FFV, FLV, FOAMV, GALV, KORV, MLVAV, MLVBM, MLVCB, MLVFF, MLVMS, PERV, SFV1, SFV3L, WMSV, and XMRV6, preferably wherein the gamma retrovirus-derived reverse transcriptase domain is not derived from PERV.

15. The artificial RNA molecule of claim 11, wherein the reverse transcriptase domain comprises one, two, three, four, five, six or more mutations corresponding to the following mutations D200N, L603W, T330P, D524G, E562Q, D583N, P51L, S67R, T197A, H204R, E302K, F309N, W313F, L435G, N454K, H594Q, L671P, E69K, or D653N in the reverse transcriptase domain of a murine leukemia virus reverse transcriptase.

16. (canceled)

17. An artificial ribonucleic acid (RNA) molecule comprising:
(1) at least one of:
(a) a 3′-untranslated region (3′UTR) element comprising a nucleic acid sequence selected from the group consisting of SEQ ID NOs: 8201-8211; and
(b) a 5′-untranslated region (5′UTR) element comprising a nucleic acid sequence selected form the group consisting of SEQ ID NOs: 8212-8247; and/or
(2) a poly(A) tail for enhancing expression of the polypeptide, wherein the poly(A) tail comprises a nucleic acid sequence selected from the group consisting of SEQ ID NOs: 8282-8314.

18-23. (canceled)

24. A system for modifying DNA comprising:
(a) an artificial ribonucleic acid (RNA) molecule for enhancing expression of a polypeptide comprising a reverse transcriptase (RT) domain and optionally an endonuclease domain, the artificial RNA molecule comprising:
(1) a nucleotide sequence encoding the polypeptide;
(2) at least one of
(i) a 3′-untranslated region (3′UTR) element for enhancing expression of the polypeptide, wherein the 3′UTR element comprises a nucleic acid sequence selected from the group consisting of SEQ ID NOs: 8201-8211, 8256, 8264-8269 and 8281; and
(ii) a 5′-untranslated region (5′UTR) element for enhancing expression of the polypeptide, wherein the 5′UTR element comprises a nucleic acid sequence selected form the group consisting of SEQ ID NOs: 8212-8247 and 8259-8263; and/or
(3) a poly(A) tail for enhancing expression of the polypeptide, wherein the poly(A) tail comprises a nucleic acid sequence selected from the group consisting of SEQ ID NOs: 8282-8314; and
(b) a template RNA (or DNA encoding the template RNA) comprising (e.g., from 5′ to 3′):
(i) optionally a sequence that binds a target site in the DNA (e.g., a second strand of a site in a target genome);
(ii) a sequence that binds the polypeptide;
(iii) a heterologous object sequence; and
(iv) optionally a 3′ target homology domain;
wherein the heterologous object sequence comprises an alteration relative to a corresponding original sequence (e.g., a wild-type sequence), wherein the alteration improves the speed, fidelity, or speed and fidelity of target-primed reverse transcription by the reverse transcriptase.

25-33. (canceled)

34. The system of claim 24, wherein the polypeptide is a heterologous gene modifying polypeptide or a retrotransposon gene modifying polypeptide.

35. The system of claim 24, wherein the polypeptide comprises the reverse transcriptase domain and the endonuclease domain.

36-42. (canceled)

43. The system of claim 24, wherein the polypeptide comprises the reverse transcriptase domain and the endonuclease domain, and the endonuclease domain is a Cas9 domain, and the template RNA comprises:
(i) a gRNA spacer that is complementary to a first portion of a target gene, and optionally comprises one or more consecutive nucleotides starting with the 3′ end of the flanking nucleotides of the gRNA spacer;
(ii) a gRNA scaffold that binds to the Cas9 domain;
(iii) a heterologous object sequence comprising a mutation region to introduce a mutation into (e.g., to correct a mutation in) a second portion of the target gene (wherein optionally the heterologous sequence comprises, from 5′ to 3′ a post-edit homology region, a mutation region, and a pre-edit homology region), and
(iv) a primer binding site (PBS) sequence comprising at least 5, 6, 7, or 8 bases with 100% identity to a third portion of the target gene.

44. The system of claim 43, wherein the target gene is a human PAH gene, and the template RNA comprises:
(i) a gRNA spacer that is complementary to a first portion of the human PAH gene, wherein the gRNA spacer has a sequence comprising the core nucleotides of a gRNA spacer sequence, and optionally comprises one or more consecutive nucleotides starting with the 3′ end of the flanking nucleotides of the gRNA spacer;
(ii) a gRNA scaffold that binds to the Cas9 domain;
(iii) a heterologous object sequence comprising a mutation region to introduce a mutation into (e.g., to correct a mutation in) a second portion of the human PAH gene (wherein optionally the heterologous object sequence comprises, from 5′ to 3′, a post-edit homology region, a mutation region, and a pre-edit homology region); and
(iv) a primer binding site (PBS) sequence comprising at least 5, 6, 7, or 8 bases with 100% identity to a third portion of the human PAH gene.

45-46. (canceled)

47. A reaction mixture comprising:
(a) a cell; or
(b) a DNA comprising a target site; and
the system of claim 24.

48-50. (canceled)

51. A deoxyribonucleic acid (DNA) molecule encoding the artificial RNA molecule of claim 1.

52. A pharmaceutical composition, comprising the artificial RNA molecule of claim 1, and a pharmaceutically acceptable excipient or carrier.

53-54. (canceled)

55. A host cell comprising the artificial RNA molecule of claim 1.

56. (canceled)

57. A method of making the artificial RNA molecule of claim 1, the method comprising synthesizing the template RNA by in vitro transcription or by introducing a DNA encoding the artificial RNA molecule into a host cell under conditions that allow for the production of the template RNA.

58. (canceled)

59. A lipid nanoparticle (LNP) comprising the artificial RNA molecule of claim 1.

60. A method for modifying a target site in genomic DNA in a cell, the method comprising: contacting the cell with the system of claim 24 or one or more nucleic acids encoding the system, thereby modifying the target site in the genomic DNA in a cell.

61. A method for treating a subject having a disease or condition associated with a genetic defect, the method comprising: administering to the subject the system of claim 24, thereby treating the subject having a disease or condition associated with a genetic defect.
